(19) United States
(12) Patent Application Publication
(10) Pub. No.: US 2019/0269764 A1
(43) Pub. Date: Sep. 5, 2019

(54) POWDER FORMULATION

(71) Applicant: Mallinckrodt Pharma IP Trading D.A.C., Dublin 15 (IE)

(72) Inventors: Laurens Adrianus Hendricus van Pinxteren, Leiden (NL); Glen Martyn, Leiden (NL); Nicola Kim Whitfield, Leiden (NL)

(21) Appl. No.: 16/408,678

(22) Filed: May 10, 2019

Related U.S. Application Data
(62) Division of application No. 14/773,212, filed on Sep. 4, 2015.
(60) Provisional application No. 61/774,125, filed on Mar. 7, 2013.
(60) Provisional application No. 61/774,143, filed on Mar. 7, 2013.

(30) Foreign Application Priority Data
Feb. 8, 2013 (GB) 1313909.2
Mar. 7, 2013 (GB) 1304145.4
Mar. 7, 2013 (GB) 1304146.2

(51) Int. Cl. A61K 38/48; A61K 9/50; A61K 9/00; A61K 9/16; A61L 26/00; C12N 9/74; A61K 9/14; A61K 38/36; C07K 14/75

(52) U.S. Cl. CPC A61K 38/4833; A61K 9/5084; A61K 9/0014; A61K 9/16; A61K 9/1623; A61L 26/0052; A61L 2400/04; A61K 9/145; A61K 38/363; C07K 14/75; C12Y 304/21005; A61L 2300/254; A61L 2300/252; C12N 9/6429

(57) ABSTRACT

The invention relates to sterile powder compositions suitable for medical use comprising thrombin and fibrinogen, and to methods for producing the same, wherein the thrombin powder is produced from a liquid feedstock, wherein the feedstock comprises a solution or a suspension of thrombin, preferably a solution, wherein the powder is produced by removal of liquid by a process selected from aseptic spray drying or aseptic fluid bed drying, and wherein the powder resulting from removal of liquid from the feedstock exhibits at least 80% of the thrombin potency or activity of the liquid feedstock, and wherein the fibrinogen powder is produced by removal of liquid from a feedstock, wherein the feedstock comprises a solution or a suspension of fibrinogen, preferably a solution, by aseptic spray drying or aseptic fluid bed drying, and wherein said composition is packaged as a sterile final pharmaceutical product for medical use.

### CROSS REFERENCE TO RELATED APPLICATIONS

This application is a divisional of U.S. application Ser. No. 14/773,212, filed Sep. 4, 2015, which claims the benefit of International Patent Application number PCT/EP2014/054477, filed Mar. 7, 2014, which claims the benefit of U.S. Provisional Application No. 61/774,125, filed Mar. 7, 2013, and U.S. Provisional Application No. 61/774,143, filed Mar. 7, 2013 the disclosures of which are hereby incorporated by reference in their entirety.

### FIELD OF THE INVENTION

This invention relates to a powder formulations suitable for use in surgery, trauma and other wounds or injuries, uses of the same and methods of making the same.

### BACKGROUND OF THE INVENTION

WO97/44015 describes a dry powder fibrin sealant based on micro-particles of fibrinogen and thrombin. Further optimised formulations of these microparticle compositions is described in co-pending application U.S. Ser. No. 12/636,718, In the Example of this application, the components are prepared by spray-drying, fibrinogen with trehalose and thrombin with trehalose. Each product has a predominant particle size of up to 50 μm in diameter. The fibrin sealant, a blend of these components, has been demonstrated to be an easy-to-use, stable and efficacious topical haemostat. The product can be used immediately, without reconstitution. On contact with aqueous fluid such as blood, the exposed and/or dissolved active thrombin converts the exposed and/or dissolved fibrinogen into insoluble fibrin polymers.

New techniques, devices, and drugs for bleeding and bleeding and/or hemorrhage control are being developed. Despite all of the technology currently available, bleeding and hemorrhage control is still a major unresolved problem in emergency medical care. Almost 50% of all deaths in the first 48 hours of hospitalization are related to an inability to adequately control bleeding. Failure to stop bleeding within the first 24 hours is almost always fatal, especially when multiple trauma sites are involved.

It is generally accepted that hemostatic products, e.g. for forward care in a battle zone, ought to control bleeding quickly, be ready to use, simple to apply, have a shelf life approaching two years and prevent bacterial or viral transmission. The product's hemostatic action may be rapid in order to meet both military and civilian needs.

Devices being investigated or used today as external methods of wound treatment range from absorbent pads containing clotting agents, pressure bandages, gauze, tourniquets for extremities, and trauma kits for wounds to the body. Agents designed to stop external bleeding differ in composition and components are designed to help the rapid formation of a clot at the site of application. Clotting products generally contain high concentrations of materials such as human fibrinogen, thrombin, calcium, factor XIII and/or anti-fibrinolytics. In addition to fibrin, microporous polysaccharide macrobeads, mineral and synthetic zeolites and chitosan (poly-N-acetyl glucosamine) are also available for use as hemostats. A number of new hemostatic products are available for treating wound trauma, for example, a bandage product using chitosan (deacetylated poly-N-acetyl glucosamine base, HemCon Inc., Tigard, Oreg.). Z-Medica Corporation, Wallingford, Conn., market a pressure bandage product (QuikClot®) for use by U.S. troops. This product uses a granular, synthetic mineral zeolite to stop bleeding by adsorbing liquid and promoting clotting. However, QuikClot® generates heat that can cause burns if the bandage isn't applied correctly.

ActSys Medical Inc., Westlake Village, Calif., provides a hemostatic gauze product, ActCel®, which is a collagen-like natural substance created from chemically treated cellulose. It expands 3 to 4 times its original size when in contact with blood, thus sealing off damaged vessels and aiding clotting.

Medafor Inc., Minneapolis, Minn., sell a bio-inert, microporous polysaccharide macrobead product that is synthesized from potatoes, called TraumaDEX®. This powdered microporous polymer product stops bleeding by expanding at the wound site and dehydrating the blood, whereupon the body absorbs the material within 48 hours.

Another non-bandage approach employs a non-zeolite topical powder containing a hydrophilic polymer and potassium salt (Quick Relief, Sarasota, Fla.) which, after application produces a flexible, protective scab to cover the wound site when the powder contacts the blood and slight pressure is applied.

No perfect solution currently exists for the treatment of excessive bleeding. Heat generation with respect to one type of agent is a major problem. The dressing's ability to adhere effectively when applied to deep wounds or wounds of irregular shape creates another major limitation. The ability to deal with excessive blood is another drawback, as is treatment and control of pressure bleeding from arterial bleeding.

Surgical and trauma wounds are the most common types of wounds addressed in the wound-care arena. Current bandages are made of gauze and are often applied in conjunction with an elastic bandage. They allow the wound to breath but are poor barriers to subsequent contamination. These bandages cannot stop serious bleeding and require the application of pressure in the case of arterial bleeding.

Conventional wound sealants fail to present an optimized combination of speed of clotting, effectiveness under pressure bleeding conditions, and clots that are dynamic over time in response to the needs of the trauma site. Typical wound sealants are usually used in conjunction with separate wound dressings.

Clearly, surgical trauma caused by sharp objects occurs in a clean environment. However, trauma wounds not caused in a controlled environment are often intermediate sized, widespread, and dirty wounds with considerable tissue damage are found in road traffic accidents or on the battlefield.

Abrasions are generally caused by scraping of the skin's outer layer; lacerations are jagged, irregular cuts or tears of the skin; punctures are caused by an object piercing the skin layers, creating a small hole; incisions are cuts commonly caused by knives or other sharp objects; and burns cause damage which may vary greatly in depth, size, and severity. Wounds due to firearms can be deep and with substantial tissue destruction. Dismemberment due to trauma requires immediate intervention to stop blood loss from the severed limb.

Liquid bandage formulations are available to the Over-the-Counter (OTC) consumer market. Liquid bandage preparations are often used for covering and protecting minor lacerations and abrasions, friction blisters and paper cuts. When applied to the skin, the solution evaporates to form a protective film over the application area and to promote healing. The polymerized film covering creates a moist wound healing environment to increase wound healing compared with conventional dressings. Most liquid bandage preparations claim to stop minor bleeding, create a protective seal over the wound, and keep out water, dirt and germs. These preparations generally act as a mechanical barrier to common microbial organisms and other forms of contamination. Liquid bandage products are available from numerous commercial sources and include New Skin Liquid Bandage®, Nexcare Bandages Spray Liquid Bandage®, Liquid Bandage by J&J, Skin Shield Liquid Bandage Curad Spray Bandage®, Powder-based hemostats are also widely available OTC in products such as QuikClot® (Z-Medica), Urgent OR® and Nosebleed OR® (BIOLIFE), TraumaDEX® and Bleed-X® (Medafor), Celox® (MEDTRADE Biopolymers), ActCel® (ActSys Medical), and Quick Relief®.

Publication WO 96/17633 describes tissue sealants including a fibrin bandage. In the method used for generating the bandage described in the WO publication, the active components are lyophilized in separate layers which are supported by an occlusive backing. Therefore, the active components are not homogeneously mixed throughout the bandage.

EP 1073485 (Zymogenetics) describes fully recombinant tissue sealant compositions, but makes no reference to dry powder forms.

However, it is frequently desirable for such products and formulations for use as tissue sealants, tissue glues, topical haemostats, wound therapies and the like, to be sterile prior to use in order to avoid contamination of a wound and the concomitant risk of infection, septicaemia etc. As such, the products and formulations need to be rendered sterile prior to packaging, or more commonly, sterilised within their final packaging.

### BRIEF DESCRIPTION OF THE FIGURES

FIG. 1 Spray dryer configuration of Example 3.

### SUMMARY OF THE INVENTION

In a first aspect of the invention, there is provided a sterile powder composition suitable for medical use comprising thrombin, wherein the powder is produced from a feedstock, such as a liquid feedstock, and wherein the powder resulting from removal of liquid from the feedstock exhibits at least 80% of the thrombin potency or activity of the feedstock.

In a second aspect of the invention, there is provided a sterile powder composition suitable for medical use comprising thrombin, wherein the powder exhibits at least 500 IU's of thrombin potency or activity per gram sterile powder composition, such as at least 600 IUs, 700 IUs, 800 IUs, 900 IUs, 1000 IUs, 1100 IUs, 1200 IUs, 1300 IUs, 1400 IUs, or more, optionally when assessed as an average of at least 3 different batches, optionally up to 10 different batches and optionally wherein the feedstock is made by dissolving or suspending a solid having an activity or potency of 1500 IU/gram solid.

In a third aspect of the invention, there is provided a sterile powder composition suitable for medical use comprising thrombin wherein the powder is produced from a feedstock, such as a liquid feedstock, and wherein the powder resulting from removal of liquid from the feedstock exhibits at least 80% of the thrombin potency or activity of the feedstock, and/or wherein the powder exhibits at least 500 IU's of thrombin activity per gram sterile powder composition, such as at least 600 IUs, 700 IUs, 800 IUs, 900 IUs, 1000 IUs, 1100 IUs, 1200 IUs, 1300 IUs, 1400 IUs, or more, optionally when assessed as an average of at least 3 different batches, optionally up to 10 different batches and optionally wherein the feedstock is made by dissolving or suspending a solid having an activity or potency of 1500 IU/gram solid, wherein the powder is produced by removal of liquid by a process selected from spray drying or fluid bed drying.

In a fourth aspect of the invention, there is provided a sterile powder composition suitable for medical use comprising thrombin wherein the powder is produced from a feedstock, such as a liquid feedstock, and wherein the powder resulting from removal of liquid from the feedstock exhibits at least 80% of the thrombin potency or activity of the feedstock, and/or wherein the powder exhibits at least 500 IU's of thrombin activity per gram sterile powder composition, such as at least 600 IUs, 700 IUs, 800, IUs, 900 IUs, 1000 IUs, 1100 IUs, 1200 IUs, 1300 IUs, 1400 IUs, or more, optionally when assessed as an average of at least 3 different batches, optionally up to 10 different batches, optionally wherein the feedstock is made by dissolving or suspending a solid having an activity or potency of 1500 IU/gram solid, wherein the powder is produced by removal of liquid by a process selected from spray drying or fluid bed drying and wherein the spray drying is an aseptic spray drying process.

In a fifth aspect of the invention, there is provided a sterile powder composition suitable for medical use comprising thrombin wherein the powder is produced from a feedstock, such as a liquid feedstock, and wherein the powder resulting from removal of liquid from the feedstock exhibits at least 80% of the thrombin potency or activity of the feedstock, and/or wherein the powder exhibits at least 500 IU's of thrombin activity per gram sterile powder composition, such as at least 600 IUs, 700 IUs, 800, IUs, 900 IUs, 1000 IUs, 1100 IUs, 1200 IUs, 1300 IUs, 1400 IUs, or more, optionally when assessed as an average of at least 3 different batches, optionally up to 10 different batches, optionally wherein the feedstock is made by dissolving or suspending a solid having an activity or potency of 1500 IU/gram solid, wherein the composition further comprises fibrinogen powder, wherein the fibrinogen powder is produced by removal of liquid from a feedstock, optionally by spray drying or fluid bed drying.

In a sixth aspect of the invention, there is provided a sterile powder composition suitable for medical use comprising thrombin wherein the powder is produced from a feedstock, such as a liquid feedstock, and wherein the powder resulting from removal of liquid from the feedstock exhibits at least 80% of the thrombin potency or activity of the feedstock, and/or wherein the powder exhibits at least 500 IU's of thrombin activity per gram sterile powder composition, such as at least 600 IUs, 700 IUs, 800, IUs, 900 IUs, 1000 IUs, 1100 IUs, 1200 IUs, 1300 IUs, 1400 IUs, or more, optionally when assessed as an average of at least 3 different batches, optionally up to 10 different batches, optionally wherein the feedstock is made by dissolving or suspending a solid having an activity or potency of 1500 IU/gram solid, wherein the composition further comprises fibrinogen powder, wherein the fibrinogen powder is produced by removal of liquid from a feedstock, optionally by spray drying, wherein said sterile powder composition comprises; (i) a composite particle comprising thrombin and fibrinogen, or (ii) a mixture of particles that comprise fibrinogen in the absence of thrombin, and particles that comprises thrombin in the absence of fibrinogen, optionally wherein the fibrinogen is co-spray dried with the thrombin to form the composition.

In a seventh aspect of the invention, there is provided a sterile powder composition suitable for medical use comprising thrombin wherein the powder is produced from a feedstock, such as a liquid feedstock, and wherein the powder resulting from removal of liquid from the feedstock exhibits at least 80% of the thrombin potency or activity of the feedstock, and/or wherein the powder exhibits at least 500 IU's of thrombin activity per gram sterile powder composition, such as at least 600 IUs, 700 IUs, 800, IUs, 900 IUs, 1000 IUs, 1100 IUs, 1200 IUs, 1300 IUs, 1400 IUs, or more, optionally when assessed as an average of at least 3 different batches, optionally up to 10 different batches, optionally wherein the feedstock is made by dissolving or suspending a solid having an activity or potency of 1500 IU/gram solid, wherein the composition further comprises fibrinogen powder, wherein the fibrinogen powder is produced by removal of liquid from a feedstock, optionally by spray drying or fluid bed drying, and wherein the fibrinogen is recombinant fibrinogen or variant thereof, such as a fibrinogen in which more than 10% of the alpha, beta or gamma chains are of a variant type, optionally wherein the variant type is preferably a gamma prime chain or an alpha extended chain.

In an eighth aspect, the invention provides a free-flowing sterile powder comprising a composition according to the invention.

In a ninth aspect, the invention provides a container comprising a sterile powder composition according to the invention.

In a tenth aspect, the invention provides a sterile powder composition packaged as a final pharmaceutical product for medical use.

In an eleventh aspect, the invention provides a kit comprising a sterile powder composition according to the invention, or a container according to the invention, optionally with a dispensing device.

In a twelfth aspect, there is provided a method of treating bleeding comprising the step of administering an effective amount of the sterile powder composition of the invention.

In a thirteenth aspect, the invention provides the use of the sterile powder composition of the invention in the manufacture of a medicament for the prevention, treatment and/or alleviation of a condition, such as wound therapy and surgical repair.

In a fourteenth aspect, the invention provides a sterile powder composition comprising thrombin wherein powder is produced from a feedstock, such as a liquid feedstock, and wherein the powder resulting from the removal of the liquid from the feedstock exhibits at least 85% or at least 90% or at least 96% of the thrombin potency or activity of the feedstock.

In a fifteenth aspect, the invention provides a sterile powder composition comprising thrombin wherein the thrombin is comprised within the powder in the form of nanoparticles, nanofibres, fibres, particles, granules, beads, microbeads, microspheres, microcapsules or microparticles, preferably microparticles.

In an sixteenth aspect, the invention provides a sterile powder composition comprising thrombin wherein the thrombin is comprised within the powder in the form of nanoparticles, nanofibres, fibres, particles, granules, beads, microbeads, microspheres, microcapsules or microparticles, preferably microparticles and wherein the composition further comprises fibrinogen powder, wherein the fibrinogen powder is produced by removal of liquid from a feedstock, optionally by spray drying or fluid bed drying, and wherein the fibrinogen is comprised within said powder in the form of nanoparticles, nanofibres, fibres, particles, granules, beads, microbeads, microspheres, microcapsules or microparticles, preferably microparticles.

In a seventeenth aspect of the invention, there is provided a sterile powder composition suitable for medical use comprising thrombin wherein the powder is produced from a feedstock, such as a liquid feedstock, and wherein the powder resulting from removal of liquid from the feedstock exhibits at least 80% of the thrombin potency or activity of the feedstock, and/or wherein the powder exhibits at least 500 IU's of thrombin activity or potency per gram sterile powder composition, such as at least 600 IUs, 700 IUs, 800, IUs, 900 IUs, 1000 IUs, 1100 IUs, 1200 IUs, 1300 IUs, 1400 IUs, or more, optionally when assessed as an average of at least 3 different batches, optionally up to 10 different batches, wherein the feedstock is a solution or a suspension of thrombin, and wherein the feedstock is selected from an aqueous or organic solvent, or a mixture thereof.

In an eighteenth aspect of the invention, there is provided a pharmaceutical composition comprising a sterile powder composition suitable for medical use comprising thrombin, wherein the powder is produced from a feedstock, such as a liquid feedstock, and wherein the powder resulting from removal of liquid from the feedstock exhibits at least 80% of the thrombin potency or activity of the feedstock, and/or wherein the powder exhibits at least 500 IU's of thrombin activity or potency per gram sterile powder composition, such as at least 600 IUs, 700 IUs, 800, IUs, 900 IUs, 1000 IUs, 1100 IUs, 1200 IUs, 1300 IUs, 1400 IUs, or more, optionally when assessed as an average of at least 3 different batches, optionally up to 10 different batches, in combination with a pharmaceutically acceptable excipient or carrier, optionally wherein the excipient is in a powder form.

In a nineteenth aspect of the invention, there is provided a pharmaceutical composition comprising a sterile powder composition suitable for medical use comprising thrombin, wherein the powder is produced from a feedstock, such as a liquid feedstock, and wherein the powder resulting from removal of liquid from the feedstock exhibits at least 80% of the thrombin potency or activity of the feedstock, and/or wherein the powder exhibits at least 500 IU's of thrombin activity or potency per gram sterile powder composition, such as at least 600 IUs, 700 IUs, 800, IUs, 900 IUs, 1000 IUs, 1100 IUs, 1200 IUs, 1300 IUs, 1400 IUs, or more, optionally when assessed as an average of at least 3 different batches, optionally up to 10 different batches, in combination with a pharmaceutically acceptable excipient or carrier, optionally wherein the excipient or carrier is a biocompatible, biodegradable polymer.

In a twentieth aspect of the invention, there is provided a pharmaceutical composition comprising a sterile powder composition suitable for medical use comprising thrombin, wherein the powder is produced from a feedstock, such as a liquid feedstock, and wherein the powder resulting from removal of liquid from the feedstock exhibits at least 80% of the thrombin activity or potency of the feedstock, and/or wherein the powder exhibits at least 500 IU's of thrombin potency or activity per gram sterile powder composition, such as at least 600 IUs, 700 IUs, 800, IUs, 900 IUs, 1000 IUs, 1100 IUs, 1200 IUs, 1300 IUs, 1400 IUs, or more, optionally when assessed as an average of at least 3 different batches, optionally up to 10 different batches, in combination with a pharmaceutically acceptable excipient or carrier, optionally wherein the excipient or carrier is a biocompatible, biodegradable polymer, wherein the excipient or carrier is present in the feedstock or is added to the powder resulting from removal of liquid from the feedstock.

In a twenty-first aspect of the invention, there is provided a pharmaceutical composition or sterile powder composition suitable for medical use comprising thrombin, wherein the powder is produced from a feedstock, such as a liquid feedstock, and wherein the powder resulting from removal of liquid from the feedstock exhibits at least 80% of the thrombin potency or activity of the feedstock, and/or wherein the powder exhibits at least 500 IU's of thrombin activity or potency per gram sterile powder composition, such as at least 600 IUs, 700 IUs, 800, IUs, 900 IUs, 1000 IUs, 1100 IUs, 1200 IUs, 1300 IUs, 1400 IUs, or more, optionally when assessed as an average of at least 3 different batches, optionally up to 10 different batches, and wherein the degree of crystallinity of said powder (optionally as measured by FTIR or XRPD) is not greater than 15% w/w, such as not greater than 10%, 8% or 7%.

In a twenty-second aspect of the invention, there is provided a pharmaceutical composition or a sterile powder composition suitable for medical use comprising thrombin, wherein the powder is produced from a feedstock, such as a liquid feedstock, and wherein the powder resulting from removal of liquid from the feedstock exhibits at least 80% of the thrombin potency or activity of the feedstock, and/or wherein the powder exhibits at least 500 IU's of thrombin activity or potency per gram sterile powder composition, such as at least 600 IUs, 700 IUs, 800, IUs, 900 IUs, 1000 IUs, 1100 IUs, 1200 IUs, 1300 IUs, 1400 IUs, or more, optionally when assessed as an average of at least 3 different batches, optionally up to 10 different batches, and wherein said powder comprises no more than 20% residual water, optionally as measured by Karl Fisher, optionally no more than 10% residual water by weight (w/w), optionally no more than 6% w/w.

In a twenty-third aspect of the invention, there is provided a pharmaceutical composition or a sterile powder composition suitable for medical use comprising thrombin, wherein the powder is produced from a feedstock, such as a liquid feedstock, and wherein the powder resulting from removal of liquid from the feedstock exhibits at least 80% of the thrombin potency or activity of the feedstock, and/or wherein the powder exhibits at least 500 IU's of thrombin activity or potency per gram sterile powder composition, such as at least 600 IUs, 700 IUs, 800, IUs, 900 IUs, 1000 IUs, 1100 IUs, 1200 IUs, 1300 IUs, 1400 IUs, or more, optionally when assessed as an average of at least 3 different batches, optionally up to 10 different batches, and wherein the powder optionally comprises 1 ug to 1000 g, such as 60 to 130 mg protein/gram composition.

In a twenty-fourth aspect of the invention, there is provided a pharmaceutical composition or a sterile powder composition suitable for medical use comprising thrombin, wherein the powder is produced from a feedstock, such as a liquid feedstock, and wherein the powder resulting from removal of liquid from the feedstock exhibits at least 80% of the thrombin potency or activity of the feedstock, and/or wherein the powder exhibits at least 500 IU's of thrombin activity or potency per gram sterile powder composition, such as at least 600 IUs, 700 IUs, 800, IUs, 900 IUs, 1000 IUs, 1100 IUs, 1200 IUs, 1300 IUs, 1400 IUs, or more, optionally when assessed as an average of at least 3 different batches, optionally up to 10 different batches, and wherein said pharmaceutical composition or powder composition additionally comprising an amorphous material, which may be a polymer, amino acid, protein or saccharide, such as trehalose, optionally wherein the material is substantially in glass form at 20 degrees C.

In a twenty-fifth aspect of the invention, there is provided a pharmaceutical composition or a sterile powder composition suitable for medical use comprising thrombin, wherein the powder is produced from a feedstock, such as a liquid feedstock, and wherein the powder resulting from removal of liquid from the feedstock exhibits at least 80% of the thrombin potency or activity of the feedstock, and/or wherein the powder exhibits at least 500 IU's of thrombin activity or potency per gram sterile powder composition, such as at least 600 IUs, 700 IUs, 800, IUs, 900 IUs 1000 IUs, 1100 IUs, 1200 IUs, 1300 IUs, 1400 IUs, or more, optionally when assessed as an average of at least 3 different batches, optionally up to 10 different batches, and wherein said pharmaceutical composition or powder composition additionally comprising an amorphous material, which may be a polymer, amino acid, protein or saccharide, such as trehalose, optionally wherein the material is substantially in glass form at 20 degrees C., wherein the material is trehalose and is present in an excess of the amount of thrombin by weight, such as in an amount of 10 to 15 times that of the thrombin by weight.

In a twenty-sixth aspect of the invention, there is provided a delivery device or container comprising a pharmaceutical composition or a sterile powder composition suitable for medical use comprising thrombin, wherein the powder is produced from a feedstock, such as a liquid feedstock, and wherein the powder resulting from removal of liquid from the feedstock exhibits at least 80% of the thrombin potency or activity of the feedstock, and/or wherein the powder exhibits at least 500 IU's of thrombin activity or potency per gram sterile powder composition, such as at least 600 IUs, 700 IUs, 800, IUs, 900 IUs, 1000 IUs, 1100 IUs, 1200 IUs, 1300 IUs, 1400 IUs, or more, optionally when assessed as an average of at least 3 different batches, optionally up to 10 different batches, optionally wherein the composition or pharmaceutical composition is approved for clinical use in humans.

In a twenty-seventh aspect of the invention, there is provided a method for preparing a sterile powder thrombin composition, the method comprising removing water from an aqueous solution comprising thrombin, wherein removing water is carried out under aseptic conditions.

In a twenty-eighth aspect of the invention, there is provided a method for preparing a sterile powder thrombin composition, the method comprising removing water from an aqueous solution comprising thrombin, wherein removing water is carried out under aseptic conditions, wherein the removal of water is by spray drying or fluid bed drying.

In a twenty-ninth aspect of the invention, there is provided a method for preparing a sterile powder thrombin composition, the method comprising removing water from an aqueous solution comprising thrombin, wherein removing water is carried out under aseptic conditions, wherein the powder is produced from a feedstock and wherein the powder resulting from the feedstock exhibits at least 80% of the thrombin activity or potency of the feedstock, optionally wherein the removal of water is by spray drying or fluid bed drying.

In a thirtieth aspect of the invention, there is provided a method for preparing a sterile powder thrombin composition, the method comprising removing water from an aqueous solution comprising thrombin, wherein removing water is carried out under aseptic conditions, optionally wherein the removal of water is by spray drying or fluid bed drying, and wherein the thrombin is provided in combination with fibrinogen.

In a thirty-first aspect of the invention, there is provided a method for preparing a sterile powder thrombin composition, the method comprising removing water from an aqueous solution comprising thrombin, wherein removing water is carried out under aseptic conditions, optionally wherein the removal of water is by spray drying or fluid bed drying, and wherein the thrombin is provided in combination with fibrinogen, wherein the fibrinogen is co-spray dried with the thrombin powder to form either; (i) a composite particle comprising thrombin and fibrinogen, or (ii) a mixture of: (a) particles that comprise fibrinogen in the absence of thrombin, and (b) particles that comprises thrombin in the absence of fibrinogen

In a thirty-second aspect of the invention, there is provided a method for preparing a sterile powder thrombin composition, the method comprising removing water from an aqueous solution comprising thrombin, wherein removing water is carried out under aseptic conditions, optionally wherein the removal of water is by spray drying or fluid bed drying, and wherein the thrombin is provided in combination with fibrinogen, wherein fibrinogen is spray dried to form a powder, thrombin is separately spray dried to form a powder and the powders are combined under aseptic conditions.

In a thirty-third aspect of the invention, there is provided a method for preparing a sterile powder thrombin composition, the method comprising removing water from an aqueous solution comprising thrombin, wherein removing water is carried out under aseptic conditions, optionally wherein the removal of water is by spray drying or fluid bed drying, and wherein the thrombin is provided in combination with fibrinogen, wherein fibrinogen is spray dried to form a powder, thrombin is separately spray dried to form a powder and the powders are combined under aseptic conditions, wherein thrombin is comprised within a microparticle, wherein fibrinogen is comprised within a microparticle and the two types of microparticle are combined in a weight ratio optionally of about 1:100 to 100:1, such as about 10:1 to 1:10, such as about 5:1 to 1:5, such as about 1:1

In a thirty-fourth aspect of the invention, there is provided a method for preparing a sterile powder thrombin composition, the method comprising removing water from an aqueous solution comprising thrombin, wherein removing water is carried out under aseptic conditions, wherein spray drying is undertaken one or both of: an atomisation pressure of at least 0.7 bar; a feed rate greater than 1300 g/hour.

In a thirty-fifth aspect of the invention, there is provided a method for preparing a sterile powder thrombin composition, the method comprising removing water from an aqueous solution comprising thrombin, wherein removing water is carried out under aseptic conditions, comprising a further step of packing the powder into a delivery device or container under aseptic conditions.

In a thirty-sixth aspect of the invention, there is provided a method for preparing a sterile powder thrombin composition, the method comprising removing water from an aqueous solution comprising thrombin, wherein removing water is carried out under aseptic conditions, wherein the powder comprises an amorphous material, which may be a polymer, amino acid, protein saccharide or, such as trehalose, optionally wherein the material is substantially in glass form at 20 degrees C.

In a thirty-seventh aspect of the invention, there is provided a sterile powder composition or pharmaceutical composition comprising thrombin, or a method of making said comprising said sterile powder composition or pharmaceutical composition comprising thrombin, said method comprising removing water from an aqueous solution comprising thrombin, wherein removing water is carried out under aseptic conditions, for use as a fibrin sealant, for example, or for use as a hemostat in the topical treatment of a wound, wherein the wound is selected from minor abrasions, cuts, scrapes, scratches, burns, sunburns, ulcers, internal venous or arterial bleeding, external venous or arterial bleeding, and surgical interventions selected from those involving the gastrointestinal system, on parenchymal organs; surgical interventions in the ear, nose and throat area (ENT) cardiovascular surgery, aesthetic surgery, spinal surgery, neurological surgery; lymphatic, biliary, and cerebrospinal (CSF) fistulae, air leakages during thoracic and pulmonary surgery, thoracic surgery, orthopaedic surgery; gynaecological surgical procedures; vascular surgery liver resection, soft tissue injury and emergency surgery.

In a thirty-eighth aspect of the invention, there is provided a pharmaceutical composition or a sterile powder composition suitable for medical use comprising thrombin, wherein the powder is produced from a feedstock, such as a liquid feedstock, and wherein the powder resulting from removal of liquid from the feedstock exhibits at least 80% of the thrombin potency or activity of the feedstock, and/or wherein the powder exhibits at least 500 IU's of thrombin activity or potency per gram sterile powder composition, such as at least 600 IUs, 700 IUs, 800, IUs, 900 IUs, 1000 IUs, 1100 IUs, 1200 IUs, 1300 IUs, 1400 IUs, or more, optionally when assessed as an average of 10 different batches, and wherein said pharmaceutical composition or powder composition additionally comprising an amorphous material, which may be a polymer, amino acid, protein or saccharide, such as trehalose, optionally wherein the material is substantially in glass form at 20 degrees C., optionally in an amount of at least 3 fold excess by weight of thrombin and, where present, at least 3 fold excess by weight of fibrinogen, optionally in 3 fold excess of the combined total of thrombin and fibrinogen.

In a thirty-ninth aspect of the invention, there is provided a pharmaceutical composition or a sterile powder composition suitable for medical use comprising thrombin, of the general type described in WO97/44105 and further optimised in co-pending application U.S. Ser. No. 12/636,718, and which additionally further comprises an additive material.

In a fortieth aspect of the invention, there is provided a pharmaceutical composition or a sterile powder composition suitable for medical use comprising thrombin, comprising a mixture of first microparticles that comprise fibrinogen, second microparticles that comprise thrombin, and further comprising an additive material.

In a forty-first aspect of the invention, there is provided a pharmaceutical composition or a sterile powder composition suitable for medical use, comprising a mixture of first microparticles that comprise fibrinogen and a stabilising excipient, second microparticles that comprise thrombin and a stabilising excipient, and further comprising an additive material. A suitable stabilising excipient is trehalose.

In a forty-second aspect of the invention, there is provided a pharmaceutical composition or a sterile powder composition suitable for medical use comprising a mixture of first microparticles that comprise fibrinogen, second microparticles that comprise thrombin, and further comprising an additive material, wherein the additive material comprises porous material.

In a forty-third aspect of the invention, there is provided a pharmaceutical composition or a sterile powder composition suitable for medical use, comprising a mixture of first microparticles that comprise fibrinogen, second microparticles that comprise thrombin, and further comprising an additive material, wherein the additive material comprises hollow material.

In a forty-fourth aspect of the invention, there is provided a pharmaceutical composition or a sterile powder composition suitable for medical use, comprising a mixture of first microparticles that comprise fibrinogen, second microparticles that comprise thrombin, and further comprising an additive material, wherein the additive material comprises a biocompatible, water-absorbent material.

In a forty-fifth aspect of the invention, there is provided a pharmaceutical composition or a sterile powder composition suitable for medical use, comprising a mixture of first microparticles that comprise fibrinogen, second microparticles that comprise thrombin, and further comprising an additive material, wherein the additive material comprises a biocompatible, water-swellable material.

In a forty-sixth aspect of the invention, there is provided a pharmaceutical composition or a sterile powder composition suitable for medical use, comprising a mixture of first microparticles that comprise fibrinogen, second microparticles that comprise thrombin, and further comprising an additive material, wherein the additive material is in the form of nanoparticles, nanofibres, fibres, particles, granules, powder, beads, microbeads, microspheres, microcapsules or microparticles, optionally wherein the additive material comprises a polysaccharide or silica.

In a forty-seventh aspect of the invention, there is provided a pharmaceutical composition or a sterile powder composition suitable for medical use comprising thrombin and fibrinogen, wherein the thrombin is produced from a liquid feedstock, wherein the feedstock comprises a solution or a suspension of thrombin, preferably a solution, wherein the thrombin is produced by removal of liquid by a process selected from aseptic spray drying or aseptic fluid bed drying, and wherein the thrombin resulting from removal of liquid from the feedstock exhibits at least 80% of the thrombin potency or activity of the liquid feedstock, and wherein the fibrinogen is produced by removal of liquid from a feedstock, wherein the feedstock comprises a solution or a suspension of fibrinogen, preferably a solution, by aseptic spray drying or aseptic fluid bed drying, and wherein said composition is packaged as a sterile final pharmaceutical product for medical use.

In a forty-eighth aspect of the invention, there is provided a method for preparing a sterile powder composition comprising fibrinogen and thrombin, wherein the thrombin powder is produced from a liquid feedstock, wherein the feedstock comprises a solution or a suspension of thrombin, preferably a solution, wherein the powder is produced by removal of liquid by a process selected from aseptic spray drying or aseptic fluid bed drying, and wherein the powder resulting from removal of liquid from the feedstock exhibits at least 80% of the thrombin potency or activity of the liquid feedstock, and wherein the fibrinogen powder is produced by removal of liquid from a feedstock, wherein the feedstock comprises a solution or a suspension of fibrinogen, preferably a solution, by aseptic spray drying or aseptic fluid bed drying, optionally wherein the composition comprises:

(i) a composite particle comprising thrombin and fibrinogen, or

(ii) a mixture of particles that comprise fibrinogen in the absence of thrombin, and particles that comprises thrombin in the absence of fibrinogen, optionally wherein (i) or (ii) are in the form of nanoparticles, nanofibres, fibres, particles, granules, beads, microbeads, microspheres, microcapsules or microparticles, preferably microparticles, and optionally wherein the composition is packaged as a sterile final pharmaceutical product for medical use, such as for direct topical application as a dry powder.

In a forty-ninth aspect of the invention, there is provided a sterile powder composition or pharmaceutical composition suitable for medical use comprising thrombin and fibrinogen, wherein administration or medical use of said composition as a hemostat in liver resection surgery or spinal surgery, elicits an anti-thrombin antibody immune response in fewer than 3%, 2%, or 1% or less in a sample population of subjects, such as where there are at least 10, 20, 30, 40, 50, 75, 100, 125, 150, 200, 300, 400, 500 or more subjects.

In a fiftieth aspect of the invention, there is provided a sterile powder composition or pharmaceutical composition comprising thrombin and fibrinogen, wherein administration or medical use of said composition as a hemostat in liver resection surgery, spinal surgery, soft tissue surgery or vascular surgery, results in a median time to hemostasis (TTH) of less than about 2 minutes, or less, such as about 1.9 minutes, or about 1.5 minutes, or about 1.0 minute.

The invention also relates to use of any of the compositions or products described herein in medicine, or in the preparation of a medicament for use in medicine, wherein the medicinal use may be the treatment of a wound, wherein the wound is selected from minor abrasions, cuts, scrapes, scratches, burns, sunburns, ulcers, internal venous or arterial bleeding, external venous or arterial bleeding, and surgical interventions selected from those involving the gastrointestinal system, on parenchymal organs; surgical interventions in the ear, nose and throat area (ENT) cardiovascular surgery, aesthetic surgery, spinal surgery, neurological surgery; lymphatic, biliary, and cerebrospinal (CSF) fistulae, air leakages during thoracic and pulmonary surgery, thoracic surgery, orthopaedic surgery; gynaecological surgical procedures; vascular surgery liver resection, soft tissue injury and emergency surgery. The invention also relates to a method of medical treatment in which the compositions or products of the invention are used in a patient in need thereof, for example a patient (human or animal) in need of treatment of conditions listed above.

### Definitions

“Active ingredient”, “pharmaceutically active agent” or “drug” as used herein means the active ingredient of a pharmaceutical, also known as an active pharmaceutical ingredient (API).

“Amorphous” as used herein may refer to a state in which a material lacks long range order at the molecular level and, depending upon temperature, may exhibit the physical properties of a solid or a liquid. In one aspect such materials do not give distinctive X-ray diffraction patterns, for example do not give a diffraction pattern from which an ordered structure can be determined. In one aspect an amorphous material may exhibit the properties of a solid, but more formally be described as a liquid. Upon heating, a change from solid to liquid properties occurs which is characterised by a change of state, typically second order (a “glass transition”).

Materials such as polymers, amino acid, proteins or saccharide, such as trehalose may each be in an amorphous state.

“Crystalline” as used herein refers to a solid phase in which the material has a regular ordered internal structure at the molecular level and gives a distinctive X-ray diffraction pattern with defined peaks. Such materials when heated sufficiently may also exhibit the properties of a liquid, but the change from solid to liquid is characterised by a phase change, typically first order (a “melting point”).

The terms “Median diameter” and “×50” [or “D (v, 0.5)” value] as used herein and which are interchangeable, refers to the median diameter (μm) of a plurality of particles, typically in a polydisperse particle population, as measured on a volume basis by a laser diffraction particle sizing system, i.e. 50% by volume of the particles are smaller than this diameter and 50% are larger. The term “×90” [or “D (v, 0.9)” value] refers to the median diameter (μm) measured on a volume basis wherein 90% of the particles are smaller than this diameter and 10% are larger. The term “×10” refers to the median diameter (μm) measured on a volume basis wherein 10% of the particles are smaller than this diameter and 90% are larger. Measuring systems include, as an example, Sympatec HELOS system H0933 (Sympatec Helos, Clausthal-Zellerfeld, Germany), or Malvern Mastersizer 2000 using the methodologies defined in any of ISO 13320-1 (1999) for laser diffraction, or Ph. Eur 2.9.31, or USP <429>. In one aspect of the present invention the particles may have a median diameter of between about 1 and 100 microns.

“Mass median aerodynamic diameter” or “MMAD” as used herein refer to the median aerodynamic size of a plurality of particles, typically in a polydisperse population. The “aerodynamic diameter” is the diameter of a unit density sphere having the same settling velocity, generally in air, as a powder and is therefore a useful way to characterize an aerosolized powder or other dispersed particle or particle formulation in terms of its settling behaviour. MMAD is determined herein by cascade impaction. In one or more embodiments, a powder of the present invention comprises a mass median aerodynamic diameter from about 1 μm to 500 μm, such as about 1.5 μm to about 400 μm, or about 2 μm to 40 μm.

“Insoluble” as used herein means having a solubility in a solvent of less than 1 mg/ml. In certain embodiments of the present invention the solubility of a material, component, excipient, pharmaceutically active agent or powder, may be less than 0.1 mg/ml, or less than 0.01 mg/ml.

“Soluble” as used herein means having a solubility in a solvent of 1 mg/ml or greater. In certain embodiments of the present invention the solubility of a material, component, excipient, pharmaceutically active agent or powder, may be greater than 10 mg/ml, or greater than 20 mg/ml. The further term, “rapidly-soluble” as used herein, means having a speed of dissolution of a proportion or all of said composition, material, component, excipient, pharmaceutically active agent or powder, in a time of less than about 20 minutes, or less than 10 minutes, or less than 5 minutes, or less than 2 minutes, or less, at room or optionally body temperature.

“Patient” as used herein refers to human or animal individuals in need of medical care and/or treatment.

“Rapidly-acting” as used herein refers to an effect achieved suitably in less than about 10 minutes, or less than about 8 minutes, or less than about 5 minutes, or less than about 4 minutes, or less than about 3 minutes, or less than about 2 minutes, or less. For example it may refer to time to hemostasis, in the case of use as a hemostat, such as wherein administration or medical use of a composition of the invention results in a time to hemostasis (TTH) of less than about 10 minutes, or less than about 8 minutes, or less than about 5 minutes, or less than about 4 minutes, or less than about 3 minutes, or less than about 2 minutes, or less.

“Wound” as used herein refers to any damage to any tissue of a patient which results in the loss of blood from the circulatory system and/or any other fluid from the patient's body. The damage may have been caused by any agent or source, including traumatic injury, infection or surgical intervention. A wound may be in a soft tissue, such as an organ, or in hard tissue, such as bone. The tissue may be an internal tissue, such as an organ or blood vessel, or an external tissue, such as the skin. The loss of blood may be internal, such as from a ruptured organ, or external, such as from a laceration.

“Resorbable material”, “absorbable carrier” and “biocompatible, biodegradable polymer” as used herein, refers to such a material, carrier or polymer that is broken down spontaneously and/or by the body into components which are degraded or eliminated without causing any significant metabolic disturbance and in such a manner as not to interfere significantly with wound healing and/or tissue regeneration.

“Solid” as used herein is intended to mean that the composition of the invention will not substantially change in shape or form when placed on a rigid surface and then left to stand at 25.degree. C. for 24 hours, and/or is not a liquid at 25.degree. C.

“Suitable” as used herein I the context of a material added to a pharmaceutical composition may mean that a material does not adversely affect the stability of the compositions of the invention or any component thereof.

For the purposes of the present invention a material, component, excipient, pharmaceutically active agent or powder is in amorphous or substantially amorphous form when it has a crystallinity of less than 15% by weight. In certain embodiments the crystallinity is suitably less than 10% by weight. In other embodiments the crystallinity is suitably less than 8% or less than 5%, for example less than 2% or less than 1%, as measured by FTIR or XRPD, or a similar suitable technique. Crystallinity may be measured for the whole powder (including an active and an additive which forms an amorphous structure), or may be considered with respect to an individual component alone, such as an excipient.

A composition in substantially glassy form (e.g. at 20 degrees C.) in one aspect may comprise up to 15% by weight crystalline content, such as up to 10% by weight crystalline content, up to 5% by weight crystalline content.

### DETAILED DESCRIPTION OF THE INVENTION

The present invention relates to materials and processes for treatment of wounds and other medical disorders. We have determined that the sterilisation processes such as irradiation applied to thrombin can cause a loss of activity of that product and have provided a process which avoids such sterilisation-induced losses in potency and which may also avoid or substantially reduce the formation of potentially immunogenic degradation products and/or aggregates. The processing conditions and the operation under aseptic conditions ensure the resultant product is of relatively high purity and also removes the need for a terminal sterilisation step altogether. The invention thus also provides for novel sterile products with higher degrees of specific activity and which are more rapidly acting than can be obtained using certain terminal sterilisation techniques in which the activity of the active is significantly decreased.

In one aspect the use of spray drying of a solution of thrombin under aseptic conditions allows a sterile product spray dried thrombin to be produced that retains high levels of the original activity or potency of the thrombin starting material, above 80%. This level can be achieved in the final form of the medicament in package form. In contrast, where thrombin is spray dried and then irradiated to sterilise the product, there is often significant loss of the thrombin activity or potency following each processing step, frequently more than 30% as a single or combined loss. As such, the use of a sterile (aseptic) spray drying process allows a thrombin powder composition to be produced which is sterile and suitable for use in patients. A sterile thrombin product, optionally additionally comprising fibrinogen, which is not sterilised by irradiation, and which retains high levels of specific activity as a result, provides advantages for wound treatment as described herein.

In one embodiment the invention relates to a sterile powder composition suitable for medical use comprising thrombin and fibrinogen, wherein the thrombin powder is produced from a liquid feedstock, wherein the feedstock comprises a solution or a suspension of thrombin, preferably a solution, wherein the powder is produced by removal of liquid by a process selected from aseptic spray drying or aseptic fluid bed drying, and optionally wherein the powder resulting from removal of liquid from the feedstock exhibits at least 80% of the thrombin potency or activity of the liquid feedstock, and wherein the fibrinogen powder is produced by removal of liquid from a feedstock, wherein the feedstock comprises a solution or a suspension of fibrinogen, preferably a solution, by aseptic spray drying or aseptic fluid bed drying, and wherein said composition is packaged as a sterile final pharmaceutical product for medical use.

In a further embodiment of the invention, there is provided a delivery device or container comprising a pharmaceutical composition or a sterile powder composition suitable for medical use, comprising thrombin and fibrinogen, wherein the thrombin powder is produced from a liquid feedstock, wherein the feedstock comprises a solution or a suspension of thrombin, preferably a solution, wherein the powder is produced by removal of liquid by a process selected from aseptic spray drying or aseptic fluid bed drying, and optionally wherein the powder resulting from removal of liquid from the feedstock exhibits at least 80% of the thrombin potency or activity of the liquid feedstock, and wherein the fibrinogen powder is produced by removal of liquid from a feedstock, wherein the feedstock comprises a solution or a suspension of fibrinogen, preferably a solution, by aseptic spray drying or aseptic fluid bed drying, and wherein said composition is packaged as a sterile final pharmaceutical product for medical use.

In one embodiment the invention relates to a sterile powder composition suitable for medical use comprising thrombin wherein the powder is produced from a feedstock, such as a liquid feedstock, and wherein the powder resulting from removal of liquid from the feedstock exhibits at least 80% of the thrombin potency or activity of the feedstock, or at least 85%, or at least 90%, or at least 95%, or more, such as at least 96% of the thrombin potency or activity of the feedstock, or at least 97%, or at least 98%, or at least 99%, or at least 99.9%.

In one aspect the removal of liquid is carried out under aseptic conditions.

In one aspect the product undergoes no subsequent sterilisation step and in one aspect may be subsequently packaged for pharmaceutical use under aseptic (sterile) conditions.

In one embodiment the invention relates to a sterile powder composition suitable for medical use comprising thrombin wherein the powder exhibits at least 500 IU's of thrombin potency or activity per gram sterile powder composition, such as at least 600 IUs, 700 IUs, 800 IUs, 900, IUs, 1000 IUs, 1100 IUs, 1200 IUs, 1300 IUs, 1400 IUs, or more, optionally when assessed as an average of at least 3 or more different batches, or at least 4, 5, 6, 7, 8, 9 or 10 different batches, optionally wherein the feedstock is made by dissolving or suspending a solid having an activity or potency of 1500 IU/gram solid.

In one embodiment the invention relates to a sterile powder composition suitable for medical use comprising thrombin wherein the powder exhibits at least 500 IU's of thrombin potency or activity per gram sterile powder composition, such as at least 600 IUs, 700 IUs, 800 IUs, 900, IUs, 1000 IUs, 1100 IUs, 1200 IUs, 1300 IUs, 1400 IUs, or more, optionally when assessed as an average of at least 3 or more different batches, or at least 4, 5, 6, 7, 8, 9 or 10 different batches, wherein each batch size is between about 0.5 grams to about 100 kg, or between about 1 grams to about 50 kg, or between about 10 grams to about 25 kg, optionally wherein the feedstock is made by dissolving or suspending a solid having an activity or potency of 1500 IU/gram solid

The sterile powder composition suitable for medical use comprising thrombin wherein the powder exhibits at least 500 IU's of thrombin potency or activity per gram sterile powder composition may be made from a feedstock as described herein.

In one aspect the starting material may have a potency or activity of more than 1500 IU/g solid, and the resulting sterile product may have an activity or potency increased in proportion (e.g. where a 1500 IU starting material results in 500 IU sterile product, a 3000 IU starting material results in 1000 IU sterile product, or more, such as a ratio of total potency or activity of an active e.g. thrombin in a feedstock material to activity in the total resulting powder being less than 1:0.5, such as 1:0.6, 1:0.7, 1:0.8, 1:0.9, 1:0.95, or even 1:1 (in the latter case wherein the production of powder from a feedstock does not result in any loss of activity and/or potency).

In one aspect the compositions of the invention are not (e.g. gamma or e-beam) irradiated before packing or in the final packaging.

In another aspect the invention provides a sterile spray dried fibrin sealant powder and/or fibrin glue and/or hemostat capable of clot formation in contact with a wound, wherein the powder has not been e-beam or gamma irradiated, optionally wherein the powder is provided in a pharmaceutically acceptable unit dose for use on a patient.

In another aspect the invention provides a sterile spray dried fibrin sealant powder and/or fibrin glue and/or hemostat comprising thrombin and fibrinogen capable of clot formation in contact with a wound when applied as a dry powder, wherein the powder has not been e-beam or gamma irradiated, optionally wherein the powder is provided in a pharmaceutically acceptable unit dose and/or device for use on a patient.

The compositions of the present invention are in the form of a powder. The term “powder” refers to two or more particles. These particles usually flow freely when shaken or tilted, but may also experience certain conglomeration forming a granular material within acceptable degrees.

In one embodiment the sterile powder composition has particles having an average diameter between 100 nm and 100 microns.

In one aspect at least 50% of the powder particles have a particle size from about 100 nm to about 100 μm when measured by standard techniques.

The sterile powder composition of the invention may comprise individual particles which are solid or hollow, such as in the case of microcapsules.

In one embodiment the sterile powder composition comprises an active—e.g. thrombin or fibrinogen in the form of particles such as nanoparticles, nanofibres, fibres, granules, beads, microbeads, microspheres, microcapsules or microparticles. Granules in this context may be defined as individual particles constituting one or more layers and/or cores, or a powder comprising a fused agglomerate of individual particles, wherein such granules or agglomerated powders may have a median diameter greater than 30, or 50 or 100 or 500 microns. Microparticles in this context may be defined as individual particles and/or separate discrete units each constituting one or more layers and/or cores, or wherein each particle and/or discrete unit comprises one or more materials as disclosed herein, optionally in a substantially homogenous state, wherein such microparticles as a population may have a median diameter of between about 1 micron and about 1 mm, or between about 5 microns and 500 microns.

The sterile powder composition of the present invention may comprise particles which are porous or non-porous.

The sterile powder composition of the present invention may be a homogeneous blend. In one embodiment the sterile powder composition comprises a fibrinogen powder which may then blended with sterile thrombin-containing powders in the form of a homogenous blend. Such blending can be carried out using low shear or high-shear blending, or any other technique known to persons skilled in the art.

The sterile powder composition in another embodiment may comprise 0.01% to 30% w/w of a thrombin, 0.01% to 30% w/w of a second active ingredient, and optionally 0.01% to 30% w/w of a third active ingredient.

The particles of the sterile powder composition of the invention suitably have a median diameter (×50) of between 1 and 2500 microns, for example of between about 5 and 500 microns or between about 10 and 40 microns.

The particles of the sterile powder composition of the invention suitably have a mass median aerodynamic diameter (MMAD) of between 10 and 2500 microns, for example of between about 5 and 500 microns or between about 10 and 30 microns.

The particles of the sterile powder composition of the invention suitably have a rugosity of greater than 1.5, for example from 1.5 to 20, 3 to 15, or 5 to 10.

In one embodiment the thrombin content or activity, as used interchangeably throughout, is suitably measured by a chromogenic assay. The chromogenic assay is a kinetic method which utilises a Chromogenix S2238™ substrate to determine the thrombin content. The substrate initiates the production of a yellow compound and the rate of change of absorbance is measured with a microplate reader at 405 nm.

This is proportional to the thrombin content of the solution. With the use of a standard curve constructed from a reference thrombin standard of known concentrations, the thrombin content in the powder can be determined.

In another embodiment of the invention, is provided a sterile powder composition suitable for medical use comprising thrombin wherein thrombin potency is suitably measured by a time to clot method, as set out in Ph. Eur, 0903 monograph for “fibrin sealant kit”.

Where the active is a protein then sources of protein for use in the invention may be naturally occurring or be made by recombinant DNA technology in cultured cells or transgenic animals or plants. The fibrinogen or thrombin may be full-length or any active fragment thereof.

Thrombin and/or any fibrinogen employed in any embodiment of the invention may be isolated from blood from human donors or be made by recombinant DNA technology in cultured cells or transgenic animals or plants.

The fibrinogen or thrombin may be full-length, wild-type (625 or 621 amino acids for fibrinogen) or any active fragment thereof. Fragments are known; see Coller et al, J. Clin. Invest. 89:546-555 (1992). Particularly suitable variant forms include variants which are the result of alternative splicing, such as the so-called gamma prime (.gamma.′ variant) and the .alpha.-ext Fib or Fib420 variant (alpha-extended). Fibrinogen raw material may be a frozen solution, although lyophilised powder which requires reconstitution prior to spray-drying may be used.

The fibrinogen may be recombinant fibrinogen or variant thereof, such as a fibrinogen in which more than 10% of the alpha, beta or gamma chains are of a variant type, wherein the variant type is preferably a gamma prime chain or an alpha extended chain.

The content or potency of thrombin in the sterile powder may be about 10 to 20,000 IU/g, or about 25 to 1000 IU/g, or 100 to 750 IU/g. The powder may comprise thrombin as the only component, or may comprise other actives or excipients.

The powder may be an intermediate suitable for combination with another powder or material, suitably to produce a sterile final composition or material.

The content of fibrinogen in the sterile powder when present, may be about 0.1 to 95% w/w, or about 0.5 to 50% w/w, or about 5 to 10% w/w, or about 6.5% w/w. The powder may comprise fibrinogen and thrombin as the only components, or may comprise other actives and/or excipients.

In certain embodiments of the invention, the sterile powder or pharmaceutical composition may have a thrombin loading of less than about 30% w/w, or less than about 10% w/w, in one aspect at least 0.05% or more by weight

The sterile powder or pharmaceutical composition of the invention may comprise between 1 ug to 1000 mg protein/gram powder or composition, such as at least 10, 50, 100, 500, or 1000 ug, 1 mg, 5, 10, 20, 30, 40, 50, 60, 70, 80, 90, 100, 125, 150, 200, 250, 500, 750 or 1000 mg protein per gram of powder or composition. In one aspect there may be 5 to 500 mg protein/gram composition or powder, such as 20 to 200, or such as 60 to 130 mg protein/gram powder or composition. The protein content may be defined as total protein for example as determined by BCA or by total clottable protein present.

The sterile powder or pharmaceutical composition of the invention, comprising fibrinogen and thrombin, may have a thrombin content as measured by a chromogenic method as disclosed herein, of between about 500 and 800 IU/g, or between about 514 and 772 IU/g, or between about 600 and 750 IU/g, or between about 620 and 700 IU/g composition.

The sterile powder or pharmaceutical composition of the invention, comprising fibrinogen and thrombin, may have a thrombin potency as measured by a time to clot method as disclosed herein, of between about 550 and 950 IU/g, or between about 581 and 908 IU/g, or between about 600 and 850 IU/g, or between about 640 and 780 IU/g composition.

The sterile powder or pharmaceutical composition of the invention, comprising fibrinogen and thrombin, may have a fibrinogen content as measured by a suitable RP-HPLC method with UV detection, of between about 50 and 90 mg/g, or between about 57 and 85 mg/g, or between about 65 and 80 mg/g, or between about 67 and 77 mg/g composition.

The sterile powder or pharmaceutical composition of the invention, comprising fibrinogen and thrombin, may have a fibrinogen potency as measured by a total clottable protein by absorption method as disclosed herein, of between about 50 and 105 mg/g, or between about 55 and 103 mg/g, or between about 60 and 85 mg/g, or between about 75 and 82 mg/g composition.

The sterile powder may comprise an additional component such as a polymer, amino acid, protein or saccharide such as trehalose, optionally wherein this material is substantially in glass form at 20 degrees C. The component may be an amorphous material and/or be in amorphous form at 20 degrees C.

The powder may comprise a component in crystalline form or the powder may be part crystalline and part amorphous in form

Therefore the invention in one aspect relates to a pharmaceutical composition or sterile powder composition comprising an amorphous material, which may be a polymer, amino acid, protein or saccharide, such as trehalose, or combination thereof, optionally wherein the material or combination of materials is substantially in glass form at 20 degrees C.

In one aspect the pharmaceutical composition or sterile powder composition according to the invention may comprise trehalose, optionally present in an excess of the amount of thrombin by weight, such as in an amount of 2, 3, 4, 5, 6, 7, 8, 9, 10 or 15 times or more than that of the thrombin by weight.

In one aspect the pharmaceutical composition or sterile powder composition according to the invention may comprise trehalose, optionally present in an excess of the amount of fibrinogen by weight, such as in an amount of 2, 3, 4, 5, 6, 7, 8, 9, 10 or 15 times or more than that of the fibrinogen by weight.

The ratio of trehalose to fibrinogen or thrombin may be determined in a powder comprising a single active, or in a powder comprising a combination of actives.

The sterile powder composition of the invention may be prepared by removal of liquid from a solution or suspension of an active component, e.g. fibrinogen or thrombin, optionally by spray drying or co-spray drying.

The removal of liquid may be from a feedstock which comprises a solution or suspension of thrombin, optionally comprising other components in solution or suspension.

In one aspect the removal of the liquid—e.g. the spray drying—is carried out in the presence of a saccharide or polyol. For example, the saccharide or polyol may be present in the feedstock.

Powders comprising fibrinogen or thrombin may be prepared by methods known in the art, for example as described in WO 92/18164, WO 96/09814, WO 96/18388 or WO 97/44015. These spray-drying and associated particle manipulation processes enable the production of e.g. protein microparticles or microcapsules with defined size distribution, for example of up to 50 μm in diameter. For example, as described in those documents, the microparticles may be produced reproducibly, e.g. with 90% or more (by volume) up to 30 μm, e.g. 10 to 20 μm, in size.

Although a suitable method of preparation of the sterile powder formulation includes spray drying, other drying techniques may also be used to prepare the sterile powder formulation. Suitable methods are known in the art and include fluidized bed drying and freeze-drying, with subsequent micronisation, or spray-freeze drying.

Sterile powders of the invention may be prepared by spray-drying. Typically, a 2-fluid nozzle is used which utilises compressed air during the atomisation process; this results in the production of hollow microparticles. The maximum particle size of microparticles (×50, as measured by Sympatec) that can be manufactured using this atomisation system on the Niro Mobile Minor™ spray dryer is approximately 30 μm.×50 values for the sterile powder or microparticles in one embodiment of the invention may be between about 5 and 50 μm, or between about 10 and 30 μm.

Spray-drying may be carried out using conventional equipment used to prepare spray dried particles for use in pharmaceuticals. Commercially available spray-dryers include those manufactured by Buchi Ltd. and Niro Corp. In its simplest form, spray drying is designed to turn a liquid feed into a dry powder in a single, continuous step. The spray drying process may comprise the following steps:

Atomisation—combination of liquid feed with gas to form droplets

Contact of droplets and drying gas—evaporation of water from the atomised droplets to form dry particles (evaporative capacity of dryer)

Powder recovery—dried particles are separated from drying airflow and collected

The strong driving force for solvent evaporation is generally provided by maintaining the partial pressure of solvent in the spray-drying apparatus well below the vapour pressure of the solvent at the temperature of the drying droplets. This may be accomplished by, (1) mixing the liquid droplets with a warm drying gas, and/or, (2) maintaining the pressure in the spray-drying apparatus at a partial vacuum (e.g., from 0.01 atm to 0.50 atm), or (3) both.

Typically, the feedstock is sprayed into a current of warm HEPA-filtered gas, such as air or Nitrogen, that evaporates the solvent and conveys the dried product to a collector. The spent air is then exhausted with the solvent. Operating conditions of the spray-dryer such as inlet and outlet temperature, feed rate, atomization pressure, flow rate of the drying air, and nozzle configuration can be adjusted in order to produce the required particle size, moisture content, and production yield of the resulting powder. Generally, the temperature and flow rate of the drying gas is chosen so that the droplets of feedstock are dry enough by the time they reach the wall of the apparatus that they may be essentially solid, form a fine powder, and do not stick to the apparatus wall. The actual length of time to achieve an appropriate level of dryness depends on the size of the droplets and the conditions at which the process is operated. Droplet sizes may range from less than 1 micron to 500 microns or more in diameter, the size being dependent on the desired particle size of the spray dried powder. The large surface-to-volume ratio of the droplets combined with the large driving force for evaporation of solvent lead to actual drying times of a few seconds or less, and often less than 0.1 second. Solidification times are often less than 100 seconds, and more often less than a few seconds.

The selection of appropriate apparatus and processing conditions are within the purview of a skilled artisan in view of the teachings herein and may be accomplished without undue experimentation. The temperature of the drying gas at the gas inlet of the spray-drying chamber may be from about 25.degree. C. to about 300.degree. C. or from about 60.degree. C. to about 250.degree. C. The spray-drying apparatus may also comprise a means for collecting the product. Specific combinations of settings may also be selected from the following: an air inlet temperature between about 60.degree. C. and about 170.degree. C., such as between 80.degree. C. and 120.degree. C.; an air outlet between about 40.degree. C. to about 120.degree. C., such as about 50.degree. C. and 100.degree. C.; a feed rate between about 1 ml/min to about 750 ml/min, optionally between about 10 and 500 ml/min; an aspiration air flow of about 0.001 to 300 L/min, optionally between about 0.01 and 100 L/min; atomization air flow rate between about 0.025 L/min and about 5 L/min. The solids content in the spray-drying feedstock may be in the range from about 0.1% w/w to 50% w/w, such as 1.0% w/w to 40% w/w. The settings may, however, vary depending on the type of equipment used, the particle size distribution desired and the nature of the solvent system employed. In any event, the use of these and similar methods allow formation of sterile powders or microparticles with diameters appropriate for use according the invention described herein.

The spray-drying may be conducted in a conventional spray-drying apparatus comprising a spray-drying chamber, atomizing means for introducing the feed mixture into the spray-drying chamber in the form of droplets, a source of heated drying gas that flows into the spray-drying chamber through an inlet, and an outlet for the heated drying gas, optionally housed in an aseptic environment. The drying air may be any gas, such as air, compressed air, or an inert gas such as nitrogen, nitrogen-enriched air, or argon and may be HEPA-filtered, i.e. sterile.

The atomizing means may be a rotary atomizer, a pneumatic nozzle, an ultrasonic nozzle or a high-pressure nozzle. Suitable rotary atomizers include those having an air turbine drive operating from a high pressure compressed air source, for example a 6 bar compressed air source, which supplies power to an atomization wheel for atomizing the feed mixture. The atomization wheel may be vaned. The rotary atomizer may be located in the upper part of the spray-drying chamber, for example in the chamber roof, so that the droplets produced dry and fall to the lower part of the chamber. Rotary atomizers produce droplets whose size depends upon the wheel peripheral velocity.

Suitable pneumatic nozzles (including two-fluid nozzles) comprise those that may be located in the upper part of the spray-drying chamber, for example in the chamber roof, and operate in so-called “co-current mode”. The feed mixture and the atomizing gas may be passed separately to the nozzle head, where the atomization takes place. The size of the droplets produced by pneumatic nozzles depends on the operating parameters.

Two-fluid nozzles that operate in so-called “counter-current mode” may also be used. These nozzles operate in a similar way to two-fluid nozzles in co-current modes except that they may be located in a lower part of the drying chamber and spray droplets upwards.

Suitable ultrasonic atomizer nozzles convert low viscosity liquids into ultra-fine sprays. As liquids are pumped through the centre of the probe, the liquids are mechanically pulverized into droplets from the vibrating tip. These droplets are larger with low frequency probes and smaller with higher frequency probes.

A suitable atomizer type for use in the invention is the high-pressure nozzle where liquid feed is pumped to the nozzle under pressure. Pressure energy is converted to kinetic energy, and feed issues from the nozzle orifice as a high-speed film that readily disintegrates into a spray as the film is unstable. The feed is made to rotate within the nozzle using a swirl insert or swirl chamber resulting in cone-shaped spray patterns emerging from the nozzle orifice. Swirl insert, swirl chamber and orifice dimensions together with variation of pressure gives control over feed rate and spray characteristics. The size of the droplets produced by high-pressure nozzles depends on the operating parameters.

Suitable atomizing means may be selected depending on the desired droplet size, which depends on a number of factors, such as the viscosity and temperature of the feed mixture, the desired flow rate and the maximum acceptable pressure to pump the feed mixture. After selecting the atomizing means so that the desired average droplet size is obtained for a feed mixture having a particular viscosity, the mixture is admitted to the spray-drying chamber at a suitable flow rate.

In an embodiment of the invention, the choice of atomiser is such that it affords protection to the feedstock and/or a smaller diameter of the liquid insert tube, where present. Protection in this context may involve a combination of a cold air sheath around the liquid feed, and/or a coolant circulating around the liquid insert tube, and/or a reduced shear stress on the liquid droplets upon atomization, for example in comparison with a Schlick two fluid nozzle.

In one aspect the atomiser nozzle is a Niro standard two fluid nozzle, or an equivalent nozzle, for example one capable of reproducing the results obtained in Table 3.

Other suitable atomisers include those developed by Ingeniatrics SA (Seville, Spain) such as the simple and concentric nebulizer, 3D Flow Focusing® nebulizer, Flow Blurring® nebulizer, high-throughput NE-4 nozzle-head, and any such Ingeniatrics atomiser set up in a multiplexed configuration providing up to 55 atomisers, for example, in operation simultaneously. Such atomisers may provide high yields of suitable powders according to the invention, whilst the Flow Focusing® atomisers may provide almost monodisperse sterile powders or microparticles and may also further demonstrate beneficial improvements in yield, flowability, etc.

In one embodiment relating to any one of the methods of the present invention, the spray-drying is performed in a spray dryer with a nozzle of a mean diameter range from around 0.1 to 10 mm, such as around 0.2 mm, 0.5 mm, 0.7 mm, 1.4 mm, or 2 mm.

Other spray-drying techniques known by the skilled in the art may be readily applied in the present invention. For example, nano spray-drying can be employed in which drying gas enters the system via the heater. A new kind of heater system allows for laminar air flow. The spray head sprays the fine droplets with a narrow size distribution into the drying chamber. The droplets dry and may become solid particles or microparticles.

In an embodiment of the invention is provided readily-flowing agglomerates comprising a pharmaceutical composition or sterile powder composition suitable for medical use comprising thrombin, optionally further comprising fibrinogen, which may be made in situ by adjusting the air flow configuration in the spray-dryer to counter-current, or arranging multiple atomisers into a “forced primary agglomeration” set-up, as would be appreciated by persons skilled in the art. Such agglomerates may have a median diameter (×50) of between 50 to 10,000 μm or 100 to 5000 μm, or 125 to 2500 μm.

The powder or particles are optionally separated in an electrostatic particle collector. The exhaust gas may be filtered and sent to a fume hood or the environment. The inlet temperature may be controlled by a temperature sensor. Other spray-drying techniques include, for instance, monodisperse spray drying technology in which conventional spray drying is combined with Microsieve™ nozzle technology to produce well-defined functional micro- and nanoparticles.

The atomizer may be a 2-fluid nozzle which uses a gas airflow to the nozzle which mixes with the liquid feedstock at the terminal tip of the nozzle to produce small, uniform droplets within the spray drying heated chamber. The nozzles may be selected or adapted such that their sheath design affords protection to the thrombin thus resulting in a greater retention of thrombin post spray drying.

In a further embodiment of the invention is the use of a separator to remove the spray dried powders from the gas stream. Suitable separators include bag collectors, cloth filters, bag filters, sintered metal filters, etc. which are available commercially from Fairey, Niro, Ohkawara, etc. The filter may be made from any suitable material, such as metal, polyester or polytetrafluoroethylene. The microparticles of the composition are retained in or on the separator, and are not collected from a cyclone, as in conventional spray dry techniques. Other suitable atomisers include pneumatic, rotary, piezoelectric, etc. Multiple orifice configurations are also suitable for use in this invention.

In a further embodiment, a three-fluid atomiser may be employed. This allows a thrombin feedstock, fibrinogen feedstock and/or any other feedstock, when present, to be delivered to the dryer from separate lines/feedstocks. Each feed may be directed to separate atomisers within the spray dryer. This may be beneficial when it is desirable to dissolve or suspend the thrombin and/or fibrinogen and/or pharmaceutically active agent, when present, in the same solvent or feedstock or in different solvents or feedstocks, and/or a further separate solvent or feedstock. For example, a dilute solution or suspension of a flow enhancer, e.g. leucine, trileucine, magnesium stearate, may be atomised separately, thereby producing an agglomerate comprising a submicron population of flow enhancer. Alternatively, a highly charged material, anionic (e.g. hyaluronic acid) or cationic (e.g. polyglutarnic acid), may be substituted for the flow enhancer.

Thrombin-containing sterile powders, and optionally fibrinogen-containing sterile powders, when present, may be formulated and blended together within a spray-drying apparatus by the use of a multi-nozzle atomizer, as described in WO03/037303, operated under aseptic conditions.

In one aspect calcium, e.g. in the form of calcium chloride or calcium lactate, may be incorporated in a feedstock e.g. the thrombin feedstock. Alternatively, calcium salts such as chloride may be added to the resulting solids after processing e.g. spray drying.

Suitable solvents from which to make a selection for use in the invention include water, ethanol, ethanol/water, acetone, dichloromethane, dimethylsulfoxide, and other Class 3 solvents as defined in ICH Q3C Guidelines, for example ICH Topic Q3C (R4) Impurities: Guideline for Residual Solvents (European Medicines Agency reference CPMP/ICH/283/95 of February 2009).

In other embodiments, the feedstock is aqueous-based, however sterile powders or microparticles of the present invention may also be prepared using pure organic solvents or bi-solvent or multi-solvent systems. Ethanol/water systems may be used as a means to control the solubility of one or more of the materials comprising the powder or microparticle.

In the present invention an active, such as fibrinogen or thrombin, or both, and optionally an excipient, may be spray-dried to give sterile powders (for example, comprising microparticles), in which the active component is in the form of a particle.

The spray dryer facility may be located within a dedicated area of a manufacturing facility, suitably in an EC GMP Grade D (Orange Guide 2007) environment, or Grade C, or Grade B or higher. The spray dryer may be contained within an EC GMP Grade A plastic isolator (ISO 5, Class 100) within this Grade D, Grade C or higher environment.

Fibrinogen and thrombin feedstock solutions may suitably be prepared in an EC GMP Grade C room. On completion of the feedstock preparation, the feedstock may be transferred into the spray dryer facility for filtration. The sterile filtered product may be collected within the Grade A isolator housing the aseptic spray dryer. The feedstocks may then be individually spray dried using the aseptic spray dryer. Post spray drying, the sterile spray-dried thrombin and/or spray-dried fibrinogen powders, when present, may be collected within the Grade A isolator and sealed as bulk powders for further processing. Blending of the two spray dried components may be performed within a Grade A isolator. The fill/finish may also suitably be conducted in a Grade A, 4 or 8-glove isolator within a Grade C cleanroom, depending on the size of the spray dryer and/or number of operatives required.

The present invention provides a sterile powder formulation that comprises the aforementioned spray-dried particles.

In another embodiment relating to an alternative method of manufacture of compositions of the present invention, is the use of spray freeze-drying technology. The sterile powders of the invention are formed by first dissolving an active such as thrombin and optionally a pharmaceutically acceptable excipient or carrier, in water. The aqueous solution is then spray freeze-dried in an inert liquefied gas such as liquid nitrogen using known techniques. (See, for example, Mumenthaler et al., Intl. J.5 Pharmaceutics (1991) 72: 97 110; Maa et al., Pharm. Res. (1999) 16:2A9-5A).

The sterile powder of the invention may comprise a saccharide or polyol, and may be prepared from a feedstock comprising a saccharide or polyol, for example may be prepared from an aqueous solution. An active such as thrombin may form part of the solution. Optionally the feedstock may comprise no other additives or actives.

The term “polyol” refers to that compound with multiple hydroxyl groups, and includes sugars (reducing and non-reducing sugars), sugar alcohols, and sugar acids. A “reducing sugar” is one which contains a hemiacetal group that can reduce metal ions or react covalently with lysine and other amino groups in proteins. A “non-reducing sugar” is one which does not have these properties of a reducing sugar. Examples of suitable polyols for the present invention include mannitol, arabitol, trehalose, sorbitol, erythritol, isomalt, lactitol, maltitol, xylitol, glycerol, lactitol, propylene glycol, polyethylene glycol, inositol, sucrose, and mixtures thereof. Sutiable polyols for use in the invention are those which are pharmaceutically acceptable. These polyols may be commercially available. Mannitol exists in three crystalline polymorphic forms: .alpha., .beta. and .delta. (see for example Burger, A., Henc, J. Rollinger, J., Weissnicht, A., Stottner, H. J. Pharm Sci, 89, 457, (2000)), and it is also found as a hemihydrate and in amorphous form.

In one embodiment relating to any one of the compositions of the present invention, the amount of polyol in respect of the drug may range from about 5 to 10 or 20 to 40 times the amount of drug (w/w).

The sterile powder composition of the present invention may comprise an excipient (which may also be referred to as an additive herein), and which combination may be referred to as a pharmaceutically composition.

Excipients may help to formulate the active components, for example, and influence biological activity.

Statements herein relating to make up of sterile powder compositions also apply to pharmaceutical compositions.

For the avoidance of doubt, sterile powder compositions may also be pharmaceutically acceptable even without an excipient or additive present.

Where produced from a feedstock, various excipients may be included in the feedstock to enhance stability, biocompatibility or other characteristics. Such excipients are also then a part of the resulting product. Excipients may include, for example, salts such as sodium chloride, buffers, chelators, surfactants such as Tween® 20, Tween® 80, Poloxamer 407 or Poloxamer 188. The use of any such excipient will be understood to those of ordinary skill in the art and the specific quantities, ratios, and types of agents can be determined empirically without undue experimentation.

Excipients may also be added to particles that have been produced by methods of the invention.

The compositions and powders of the invention may also comprise carrier materials which may be biologically inert but can influence, for example, the effectiveness of drug delivery.

In one embodiment relating to any one of the compositions of the present invention, the pharmaceutically acceptable excipients or carrier, or amorphous material or polyol, and thrombin, are present in a ratio (w/w, respectively) of ranges from around 1000/1 to around 1/1000, or in a ratio from around 100/1 to around 1/100, even in a ratio from around 50/1 to around 1/50.

In one embodiment relating to any one of the compositions of the present invention, the pharmaceutically acceptable excipients or carrier, or amorphous material or polyol, and fibrinogen, when present, are present in a ratio (w/w, respectively) of ranges from around 1000/1 to around 1/1000, or in a ratio from around 100/1 to around 1/100, even in a ratio from around 50/1 to around 1/50.

In one embodiment the additive material is present in the form of discrete particles that are separate from the sterile particles comprising fibrinogen and thrombin. Thus, the additive material may be in the form of nanoparticles, nanofibres, fibres, particles, granules, powder, beads, microbeads, microspheres, microcapsules or microparticles.

In one embodiment the additive material typically has an average particle size and/or median diameter (×50) of from 10 to 1000 μm, or 100 to 500 μm, or 125 to 250 μm or possibly, for example, 10 to 40 μm. The additive may comprise one material or may be a mixture of materials. Such additive material may act as a carrier and/or diluent for the active materials (fibrinogen and thrombin) present as a sterile powder.

Additive materials that may be present in the form of particles having particle sizes of the order set out in the preceding paragraph include such additives as biocompatible water-absorbent and/or water-swellable materials, polysaccharides, porous and/or hollow materials.

Typically, the composition in such cases may comprise at least 1%, or at least 5% or at least 10% w/w of additive material, and up to 60%, up to 70% or up to 80% w/w of additive material. Thus, the additive may be present at a level of from 1% (or 5% or 10%) to 80%, or from 1% (or 5% or 10%) to 70%, or from 1% (or 5% or 10%) to 60% w/w of the composition.

In other embodiments of the invention, the additive material typically has an average particle size and/or median diameter (×50) of from 10 nm to 1000 μm, or between about 1 and 500 microns, and may comprise one material or may be a mixture of materials.

Additive materials that may be used in the form of particles having particle sizes of the order set out in the preceding paragraph include silica, in particular hydrophilic fumed silica.

The additive material may comprise about 0.01%, about 0.05%, about 0.1%, about 0.5%, about 1%, about 2%, about 3%, about 4% about 5%, about 6%, about 7%, about 8%, about 9%, about 10%, about 11%), about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, by weight of the powder or composition, or any range between.

Typically, the composition in such cases may comprise at least 0.001%, or at least 0.01% or at least 0.05% w/w of additive material, and up to 0.1%, up to 0.5% or up to 1% w/w of additive material. Thus, the additive may be present at a level of from 0.001% (or 0.01% or 0.05%) to 1%, or from 0.001% (or 0.01% or 0.05%) to 0.5%, or from 0.001% (or 0.01% or 0.05%) to 0.1% w/w of the composition.

Various materials are suitable for use as a biocompatible, water-absorbent and/or water-swellable additive material, for enhancing flow and wettability, etc. The material may be insoluble or very slowly soluble. Such materials may include dextran polymers, e.g. Sephadex, which are available in different particle sizes, starches including hetastarch, pullulan derivatives, hyaluronic acid esters, cellulose products such as microcrystalline cellulose (Avicel range), methylcellulose, carboxymethyl cellulose, microfine cellulose or hydroxy propyl cellulose, hydroxypropylmethylcellulose, hydroxypropylcellulose, low-substituted hydroxypropyl cellulose, hydroxyethylcellulose and other materials such as cross-linked polyvinyl pyrrolidone (PVP), may be used singly or in admixture. Also, suitable additive materials acting as carriers include polyethylene glycol (PEG), which may have a molecular weight of about 1000; polyvinylpyrrolidone (PVP), which may have an average molecular weight of about 50,000; Poly(acrylic acid), polyacrylamide, poly vinyl alcohol (PVA), Poly(methylvinylether co-maleic anhydride), Poly(ethyleneoxide), and dextran, typically having an average molecular weight of about 40,000.

Additive materials may be sterile or sterilised (by e.g. electron beam irradiation, .gamma.-irradiation and ethylene oxide, etc.), and subsequently incorporated by sterile processing.

In certain embodiments of the invention, the additive material is a silica, such as a hydrophilic silica. Such silicas may be colloidal silicas, fumed silicas, ground silicas, precipitated silicas, or mixtures thereof. Examples of suitable fumed silicas include, but are not limited to, Aerosil® 90, 130, 150, 200, 300, 380, R202, R805, R812, R972, R974 (Degussa Corporation, Ridgefield Park, N.J.) and CAB-O-SIL® TS-720 and M-5 (Cabot Corporation, Tuscola, Ill.). Generally, Aerosil® 200, Aerosil® R974, CAB—O-SIL® TS-720 and any other generally equivalent products from other manufacturers of fumed silicas may be suitable.

It is known that hydrophilic AEROSIL® colloidal silica increases the rate of tablet disintegration and active ingredient release. The colloidal silica acts as a “wick” to draw the water—for example from the digestive juices—into the interior of the tablet. Moreover, tablet ingredients “coated” with hydrophilic AEROSIL® 200 colloidal silica are more easily wetted and swell faster (disintegrants) or dissolve faster (active ingredient). Such properties enhance the wettability and dissolution of the powdered fibrin sealant of the instant invention. Furthermore, such silicas are known to act as glidants, and so may enhance the flowability, filling and delivery of such cohesive microparticulates. Moreover, such colloidal silicas are known activators for blood clotting and thus act synergistically with the fibrinogen and thrombin components (see Margolis, “The Effect of Colloidal Silica on Blood Coagulation”, Aust. J. Exp. Biol., 39, pp. 249-258 (1961)).

The sterile powder composition may comprise from about 0.001 to 5% w/w, about 0.01 and 2% w/w, or about 0.01 to 0.5% w/w of a silica. The silica may be simply blended with a fibrinogen-containing component and then a thrombin-containing component added thereto and blended further under aseptic conditions, or vice versa. The silica may be blended with the pre-blended thrombin-containing and fibrinogen-containing sterile powder when present, as a final step. Suitable blending apparatus will be known to those skilled in the art.

In a further embodiment, the silica may be present in combination with a further carrier and/or additive material, as defined herein.

In other embodiments of the invention, the additive may be a highly porous and highly soluble interwoven filamentary crystal, e.g. of sorbitol and/or mannitol. Such materials are sold under the name PARTECK SI and PARTECK M (Merck KGaA, Darmstadt, Germany). These grades have a high adsorption capacity and so are suitable for blending with the dry powder fibrin sealant powder composition of the invention, to produce a novel powder which may reduce dusting, enhance wettability, solubilisation and performance of the sterile powder composition of the invention, by allowing blood to soak through the applied powder bed and thus avoid clotting at the powder interface alone.

The additive materials may be present in any compositions of the invention as single components or in combination and may be present in the feedstock or added as an material to either dried, e.g. spray-dried, thrombin or fibrinogen component before blending together, or added to the final blend and subjected to further blending. Such blending can be carried out using low shear or high-shear blending, mechano-chemical bonding, hybridisation or any other technique known to persons skilled in the art.

Additives including excipients or carriers may be added to prepared particles or sterile powders of active, such as thrombin or fibrinogen, or may be added to a feedstock used to prepare particles or sterile powders of active such as thrombin or fibrinogen particles.

In one or more embodiments of the sterile powder composition of the present invention, addition of a pharmaceutically acceptable excipient or carrier may act to enhance stability or biocompatibility of the formulation.

In one aspect the pharmaceutically acceptable excipient or carrier is present in amorphous form or phase within the sterile powder or microparticle. Said amorphous form or phase excipient may be selected from glass-forming materials such as sugars (e.g., sucrose, trehalose, lactose), polyols, sugar alcohols (e.g., mannitol), amino acids/peptides (e.g., leucine), and salts/buffers (e.g., sodium citrate, sodium maleate, calcium lactate).

Multiple components in a powder or pharmaceutical composition according to the invention may be in amorphous form.

Suitable glass-forming excipients may be those with a glass transition temperature, T.sub.g, greater than about 100 degrees C. (e.g., sodium citrate, inulin, and trehalose). Such glass-forming polyols may be referred herein as stabilizing polyols. Water-soluble glass-forming excipients may be chosen such that they may dissolve and/or diffuse rapidly away upon administration.

Thus the invention relates to any composition disclosed herein comprising thrombin and/or fibrinogen in combination with a glass-forming sugar (e.g., sucrose, trehalose, lactose), polyol, sugar alcohols (e.g., mannitol), amino acids/peptide (e.g., leucine), and salts/buffers (e.g., sodium citrate, sodium maleate, calcium lactate), and/or in combination with a glass-forming excipient with a T.sub.g greater than about 100 degrees C. (e.g., sodium citrate, inulin, and trehalose).

In one aspect the invention also relates to a method for preparing a sterile powder thrombin composition, the method comprising removing water from an aqueous solution comprising thrombin, wherein removing water is carried out under aseptic conditions, wherein the resulting powder comprises a polymer, amino acid, protein or saccharide such as trehalose, optionally wherein the material is substantially in glass form at 20 degrees C., and in another aspect is an amorphous material.

The amount of the glass-forming material or excipient which maybe present may be determined by the glass transition temperatures of the drug substance to be stabilized, and the glass-forming material or excipient itself. An optional T.sub.g for the sterile powder or microparticle of the invention may be at least 50 or at least 60 or at least 70 or at least 80.degree. C. The Fox equation can be utilized to estimate the quantity of glass-forming excipient required to achieve this value, viz:

$$\frac{1}{{T}_{g}} = \frac{{w}_{1}}{{T}_{g ( 1 )}} + \frac{{w}_{2}}{{T}_{g ( 2 )}}$$

Where i and .sup.w2 are the weight fractions of the drug and glass forming excipient, respectively. Table 1 provides a list of common glass-forming materials, and their representative dry T.sub.g values.

TABLE 1
Dry Tg values of some common
glass-forming excipients and related materials

| Excipient | Dry T_{g} (° C.) |
|---|---|
| glycerol | −93 |
| sorbitol | −3 |
| fructose | 13 |
| glucose | 38 |
| maltose | 101 |
| sucrose | 73 |
| trehalose | 117 |
| raffinose | 104 |
| lactose | 112 |
| mannitol | 11 |
| sodium citrate | 170 (pH > 7) |
| maltohexose | 173 |
| leucine | 140 |
| trileucine | 70-100 (pH dependent) |

An additive material may also be in a crystalline or amorphous state and may also be free-flowing, and/or discrete and/or substantially anhydrous, optionally with a residual moisture or water content no greater than about 5% w/w, or no greater than about 3% w/w.

The additive material may also be amorphous or in the form of a glass at room temperature (e.g. 20.degree. C. or 25.degree. C.) and may optionally be in a rapidly soluble state. The additive may exhibit a glass transition temperature (Tg) of greater than about 25.degree. C., or about 30.degree. C., or about 40.degree. C., or about 50.degree. C., as measured by Differential Scanning calorimetry or modulated Differential Scanning calorimetry.

In an embodiment of the invention, a pharmaceutical composition or sterile powder composition suitable for medical use comprising thrombin and fibrinogen, may be substantially amorphous and/or may be in the form of a glass at room temperature (e.g. 25.degree. C.) which may stabilise the entrapped thrombin and/or fibrinogen and/or protein and/or pharmaceutically-active agent when present, for extended periods of time, for example greater than or up to one to two years, or more, with no more than about 1%, or about 2%, or about 3%, or about 4%, or about 5% or about 6% or about 7% loss in activity and/or potency over a two year period.

In an embodiment of the invention, a pharmaceutical composition or sterile powder composition suitable for medical use comprising trehalose, thrombin and fibrinogen, may be substantially amorphous and/or may be in the form of a glass at room temperature (e.g. 25.degree. C.) which may stabilise the entrapped thrombin and/or fibrinogen and/or protein and/or pharmaceutically-active agent when present, for extended periods of time, for example greater than or up to one to two years, or more, with no more than about 1%, or about 2%, or about 3%, or about 4%, or about 5% or about 6% or about 7% loss in activity and/or potency over a two year period.

In one embodiment of the invention there is provided a pharmaceutical composition or a sterile powder composition suitable for medical use comprising thrombin, of the general type described in WO97/44105 and further optimised in co-pending application U.S. Ser. No. 12/636,718, and which additionally further comprises an additive material.

In another embodiment of the invention, there is provided a pharmaceutical composition or a sterile powder composition suitable for medical use comprising thrombin, comprising a mixture of first microparticles that comprise fibrinogen, second microparticles that comprise thrombin, and further comprising additive material.

In another embodiment of the invention, there is provided a pharmaceutical composition or a sterile powder composition suitable for medical use, comprising a mixture of first microparticles that comprise fibrinogen and a stabilising excipient, second microparticles that comprise thrombin and a stabilising excipient, and further comprising additive material.

The sterile powder of the invention may be prepared by spray-drying a solution of the active component, i.e. fibrinogen or thrombin, optionally with a saccharide or polyol alone, and optionally wherein the powder constitutes a blend of both active-containing materials, trehalose is the sole polyol present in either or both of the thrombin-containing or fibrinogen-containing sterile powders.

Compositions of the present invention may comprise a pharmaceutically active agent such as an amino acids, peptides, and proteins; antibodies; biological factors such as antigens, blood coagulation factor inhibitors, blood coagulation factors, chemotactic factors, inflammation mediators, intercellular signaling peptides and proteins, pheromones, biological pigments, biological toxins; enzymes and coenzymes; hormones, hormone substitutes, and hormone antagonists; macromolecular substances such as micelles, multiprotein complexes, and polymers; nucleic acids, nucleic acid precursors, antisense elements, nucleotides, nucleosides; antitoxins such as antivenins, diphtheria antitoxin, and tetanus antitoxin; immune sera such as antilymphocyte serum; menotropins; nectar; picibanil; vaccines including and without being limited to Alzheimer vaccines, bacterial vaccines, cancer vaccines, fungal vaccines, protozoan vaccines, toxoids, attenuated vaccines, combined vaccines, contraceptive vaccines, inactivated vaccines, marker vaccines, viral vaccines; as well as adjuvants, Toll-like receptor (TLR) ligands, pattern recognition receptor ligands, cytokines, and the like. Other suitable additional pharmaceutically-active agents for use as described herein include growth factors, polyclonal and monoclonal antibodies, drugs, and other compounds including, but not limited to, the following: fibrinolysis inhibitors, such as aprotonin, tranexamic acid and epsilon-amino-caproic acid; antibiotics, such as tetracycline and metronidazole, ciprofloxacin and amoxicillin; anticoagulants, such as activated protein C, prostaglandins (particularly (PGI.sub.2), leukotrienes, heparin, ADPase, prostacyclins, antithrombin III, and plasminogen activator; steroids, such as dexamethasone, inhibitors of prostacyclin, prostaglandins, leukotrienes and/or kinins to inhibit inflammation; cardiovascular drugs, such as calcium channel blockers, vasodilators and vasoconstrictors; local anaesthetics such as bupivacaine; and antiproliferative/antitumor drugs such as 5-fluorouracil (5-FU), Taxol® (paclitaxel) and/or Taxotere® (docetaxel); anti-virals, such as gancyclovir, zidovudine, amantidine, trifluridine, acyclovir, vidarabine, ribaravin, dideoxyuridine and antibodies to viral components; mammalian gene products; cytokines, such as alpha- or beta-tumour necrosis factor, alpha- or beta- or gamma-Interferon, and interleukins; colony stimulating factors; erythropoietin; antifungals, such as Diflucan® (fluconazole), ketaconazole and nystatin; antiparasitic gents, such as pentamidine; anti-inflammatory agents, such as alpha-1-anti-trypsin and alpha-1-antichymotrypsin; anaesthetics, such as bupivacaine; analgesics; antiseptics; hormones; vitamins and other nutritional supplements; glycoproteins; fibronectin; peptides and proteins; carbohydrates (both simple and/or complex); proteoglycans; antiangiogenins; antigens; lipids or liposomes; oligonucleotides (sense and/or anti-sense DNA and/or RNA); and gene therapy reagents.

Such pharmaceutically active agents may be combined with thrombin and/or fibrinogen.

One or more drugs or pharmaceutically-active agents which may be present in the sterile powder or compositions of the invention may be selected from amino acids, peptides, proteins, antibodies, antigens, nucleic acids, nucleic acid precursors, antisense elements, nucleotides, nucleosides, antitoxins, vaccines, and adjuvants.

In an embodiment of the invention, a pharmaceutical composition or sterile powder composition suitable for medical use comprising thrombin, optionally further comprising fibrinogen, may be substantially amorphous and/or may be in the form of a glass at room temperature (e.g. 25.degree. C.) which may stabilise the entrapped thrombin and/or fibrinogen and/or protein and/or pharmaceutically-active agent when present, as well as may enable the powder or composition to be presented in a rapidly-soluble and/or rapidly-acting state.

The pharmaceutical composition or sterile powder composition may exhibit a glass transition temperature of greater than about 25.degree. C., or about 30.degree. C., or about 40.degree. C., or about 50.degree. C., as measured by Differential Scanning calorimetry or modulated Differential Scanning calorimetry. In one aspect such a glass composition may be stored at ambient or room temperature, e.g. 25.degree. C., for extended periods of time, for example greater than or up to 3 months or greater than or up to 6 months, or about up to one to two years, or more, without significant losses in activity of the incorporated pharmaceutically-active agent(s), e.g. no more than about 10%, or about 15% or about 20% loss in activity and/or potency over that time.

In one embodiment relating to any one of the compositions of the present invention, the components are homogeneously distributed, for example the pharmaceutically acceptable excipient or carrier (e.g. in amorphous form), and the thrombin and/or fibrinogen and/or other pharmaceutically-active agents, when present, are homogeneously distributed.

The term “homogeneously distributed” means that the components occur at a substantially constant ratio to each other in any given area or region of the composition such that a comparison of the analyses of a selection of samples for potency, bioactivity or such like, from within the sterile powder or composition is such that the percent coefficient of variation (% CV) of measured activity, and/or content and/or potency may optionally be less than about 20 percent or less than about 10 percent or less than about 5 percent, or less.

In certain embodiments, a sterile powder composition or pharmaceutical composition may contain areas of higher or lower concentration of the components. The term “components” refers to any material, e.g. amorphous material, polyol, and/or thrombin, and/or fibrinogen, and/or pharmaceutically-active agent.

In one embodiment an amorphous material and one or more of thrombin and/or fibrinogen and/or pharmaceutically-active agent, are homogeneously distributed in any composition of the invention.

In an embodiment of the invention, the sterile powder composition or pharmaceutical composition comprising thrombin may be substantially free of small soluble protein aggregates (e.g., subvisible aggregates) of less than about 0.2 or about 0.1 microns in size, as determined, for example, by size exclusion chromatography (SEC). The sterile powder composition or pharmaceutical composition comprising thrombin may be greater than 90%, or 95%, or 97%, or 98%, or 99% monomeric protein, or in some embodiments may be substantially chromatographically pure as determined by SEC.

The immunogenic potential of the compositions of the invention may be determined and/or quantified by methods measuring the ability of the composition of the invention to elicit antibodies in a suitable animal model and/or human population.

A sterile powder composition or pharmaceutical composition of the invention may further be characterized for aggregate or particulate content by one or a plurality of additional analytical techniques selected from ultracentrifugation, size-exclusion chromatography and gel permeation chromatography, field flow fractionation, dynamic light scattering, light obscuration or fluorescence spectroscopy:

The above methods are applicable analysis of thrombin and or fibrinogen containing compositions as disclosed herein.

In one embodiment, the composition or powder of the invention is optionally substantially free of aggregates, optionally in the form selected from dimers, trimers or oligomers, as measured by SEC.

In one embodiment, the composition or powder of the invention optionally contains not more than about 0.01 or about 0.2 or about 0.5 or about 1 or about 2 or about 5 or about 10 percent by weight aggregates, optionally in the form selected from dimers, trimers or oligomers.

In one embodiment, the composition or powder of the invention comprising fibrinogen and thrombin, optionally contains not more than about 0.01 or about 0.2 or about 0.5 or about 1 or about 2 or about 5 or about 6, or about 7, or about 10 percent by weight aggregates, optionally in the form selected from dimers, trimers or oligomers, as measured by HP-SEC.

In one embodiment, the composition or powder of the invention comprising thrombin, optionally contains not more than about 10 percent, or about 11 percent, or about 12, or about 13 percent, or about 14 percent by weight aggregates, optionally in the form selected from dimers, trimers or oligomers, as measured by HP-SEC.

Many methods of gel electrophoresis (e.g., denaturing or non-denaturing PAGE) can be employed to analyze proteins and protein aggregation. Native PAGE (non-denaturing PAGE) can be used to study non-covalently linked aggregates. See, e.g., Hermeling et al. J. Phar. Sci. 95: 1084-1096 (2006); Kilic et al., Protein Sci. 12:1663 (2003); Westermeier, R., Electrophoresis in Practice: A Guide to Methods and Applications of DNA and Protein Separations 4th edition, New York: John Wiley & Sons, 2005; and Hames, B. D. (Ed.), Gel Electrophoresis of Proteins: A Practical Approach, 3rd edition, New York: Oxford University Press, USA, 1998.

As used herein, a “protein aggregate” or “protein particulate” is defined as being composed of a multiplicity of protein molecules wherein non-native non-covalent interactions and/or non-native covalent bonds (such as non-native intermolecular disulfide bonds) hold the protein molecules together. The aggregates may be soluble or insoluble. Protein aggregates include, but are not limited to, inclusion bodies, soluble and insoluble precipitates, soluble non-native oligomers, gels, fibrils, films, filaments, protofibrils, amyloid deposits, plaques, and dispersed non-native intracellular oligomers.

The invention further relates to a sterile powder composition suitable for medical use comprising thrombin and fibrinogen wherein the powder is produced from a feedstock wherein the feedstock comprises a solution or a suspension of thrombin, preferably a solution, wherein the powder is produced by removal of liquid by a process selected from aseptic spray drying or aseptic fluid bed drying, and wherein the powder resulting from removal of liquid from the feedstock exhibits at least 80% of the thrombin potency or activity of the liquid feedstock, or even at least 85%, or even at least 90%, or even at least 95% or more as measured by a time to clot method, as set out in Ph. Eur, 0903 monograph for “fibrin sealant kit”, and wherein the composition further comprises fibrinogen powder produced by removal of liquid from a feedstock, wherein the feedstock comprises a solution or a suspension of fibrinogen, preferably a solution, by aseptic spray drying or aseptic fluid bed drying.

The sterile powder composition or pharmaceutical composition of the invention comprising thrombin and/or fibrinogen may be of high monomer content (for example, at least 80% monomer; at least about 90% monomer; at least about 95% monomer; at least about 97% monomer, at least about 98% monomer, or at least about 99% monomer).

The sterile powder composition or pharmaceutical composition of the invention comprising thrombin and/or fibrinogen may be of high non-denatured content (for example, at least 80% non-denatured; or at least about 90% non-denatured; or at least about 95% non-denatured; at least about 97% non-denatured, at least about 98% non-denatured, or at least about 99% non-denatured).

The invention relates to a sterile powder composition suitable for medical use comprising thrombin wherein the powder is produced from a feedstock, such as a liquid feedstock, and wherein the powder resulting from removal of liquid from the feedstock exhibits at least 80% of the thrombin potency or activity of the feedstock, or even at least 85%, or even at least 90%, or even at least 95%, or more, as demonstrated by maintenance of band intensity, relative to control, when assayed or measured by Western blot.

The invention relates to a sterile powder composition suitable for medical use comprising thrombin wherein the powder is produced from a feedstock, such as a liquid feedstock, and wherein the powder resulting from removal of liquid from the feedstock exhibits at least 80% of the thrombin potency or activity of the feedstock, or even at least 85%, or even at least 90%, or even at least 95% or more, whereby no or little band spreading/smearing is observed as measured by non-reduced SDS-PAGE, using a non-specific protein stain, e.g. Coomassie Blue.

The invention relates to a method for preparing a sterile powder thrombin composition, the method comprising removing water from an aqueous solution comprising thrombin, wherein removing water is carried out under aseptic conditions, optionally wherein said sterile powder composition or pharmaceutical composition comprising thrombin has reduced immunogenicity.

The products, sterile powder compositions and pharmaceutical compositions as described herein, provide certain advantages with respect to their immunogenicity when administered to patients. Therefore the invention also relates to the following aspects:

A sterile powder composition suitable for medical use comprising thrombin wherein the powder composition elicits an anti-thrombin antibody immune response in fewer than 5% (4%, 3%, 2%, 1%) of patients.

In one aspect the anti-thrombin antibody immune response is measured by a validated ELISA assay. In one aspect there is no detectable neutralising antibody response, for example, as determined by methods disclosed herein.

In one aspect the sterile powder composition or pharmaceutical composition comprises an additional pharmaceutically active agent, such as fibrinogen.

In another aspect the sterile powder composition or pharmaceutical composition comprises, or is used in a manner disclosed in any other aspect herein, for example any claim herein, for example:

the powder is produced from a feedstock, such as a liquid feedstock, and wherein the powder resulting from removal of liquid from the feedstock exhibits at least 80% of the thrombin potency or activity of the feedstock;

the powder exhibits at least 500 IU's of thrombin potency or activity per gram sterile powder composition, such as at least 600 IUs, 700 IUs, 800, IUs, 900 IUs, 1000 IUs, 1100 IUs, 1200 IUs, 1300 IUs, 1400 IUs, or more, optionally when assessed as an average of at least 3 different batches, optionally up to 10 different batches, optionally wherein the feedstock is made by dissolving or suspending in a liquid a solid having an activity or potency of 1500 IU/gram solid to produce the feedstock;

the powder is produced by removal of liquid by a process selected from spray drying or fluid bed drying, e.g. the spray drying is an aseptic spray drying process.

The powder comprises

(i) a composite particle comprising thrombin and fibrinogen, or

(ii) a mixture of [0255] particles that comprise fibrinogen in the absence of thrombin, and

particles that comprises thrombin in the absence of fibrinogen optionally wherein the fibrinogen is co-spray dried with the thrombin to form the composite or mixture;

the powder composition is packaged as a sterile final pharmaceutical product for medical use;

the thrombin is comprised within the powder in the form of nanoparticles, nanofibres, fibres, particles, granules, beads, microbeads, microspheres, microcapsules or microparticles, preferably microparticles;

the powder composition is a pharmaceutically acceptable comprises a pharmaceutically acceptable excipient or carrier, optionally wherein the excipient is in a powder form;

the pharmaceutical composition or powder composition has a degree of crystallinity as measured by XRPD or FTIR not greater than 15% w/w, such as not greater

The invention relates to a method for preparing a sterile powder thrombin composition, the method comprising removing water from an aqueous solution comprising thrombin, wherein removing water is carried out under aseptic conditions, optionally wherein the conditions are selected to favour and/or preserve monomeric protein, such as greater than 90%, or greater than 95%, or greater than 97% monomeric material as determined by size exclusion chromatography, optionally wherein said sterile powder composition or pharmaceutical composition comprising thrombin has reduced immunogenicity.

In another aspect the immunogenicity may be assessed in an vivo analysis on human or non-human trials.

In a particularly preferred embodiment is provided a sterile powder composition or pharmaceutical composition comprising thrombin according to the invention, optionally further comprising fibrinogen, for use as a hemostat in liver resection, spinal surgery, soft tissue surgery or vascular surgery, wherein administration or medical use of said composition elicits an anti-thrombin antibody immune response in fewer than 3%, 2%, 1% or less in a sample population of subjects, such as where there are at least 10, 20, 30, 40, 50, 75, 100, 125, 150, 200, 300, 400, 500 or more subjects. In this embodiment, subjects may be considered antibody positive if they have seroconverted (i.e. negative at baseline with a specific and measurable titer at day 29 post-administration) or had a greater than 1.0 titer unit change at Day 29 post-administration, and/or subjects who were negative at baseline with a specific and measurable titer at Day 29 post-administration.

In a further particularly preferred embodiment is provided a sterile powder composition or pharmaceutical composition comprising thrombin according to the invention, optionally further comprising fibrinogen, for use as a hemostat in liver resection surgery or spinal surgery, wherein administration or medical use of said composition elicits an anti-thrombin antibody immune response in fewer than 3%, 2%, or 1% or less in a sample population of subjects, such as where there are at least 10, 20, 30, 40, 50, 75, 100, 125, 150, 200, 300, 400, 500 or more subjects. In this embodiment, subjects may be considered antibody positive if they have seroconverted (i.e. negative at baseline with a specific and measurable titer at day 29 post-administration) or had a greater than 1.0 titer unit change at Day 29 post-administration, and/or subjects who were negative at baseline with a specific and measurable titer at Day 29 post-administration.

In a particularly preferred embodiment of the invention is the use of a sterile powder composition or pharmaceutical composition comprising thrombin according to the invention, optionally further comprising fibrinogen, for use in the treatment of bleeding associated with liver resection surgery, spinal surgery, soft tissue surgery or vascular surgery.

In another particularly preferred embodiment of the invention is the use of a sterile powder composition or pharmaceutical composition comprising thrombin according to the invention, optionally further comprising fibrinogen, for use in the treatment of bleeding associated with liver resection surgery or spinal surgery.

In a further particularly preferred embodiment is provided a sterile powder composition or pharmaceutical composition comprising thrombin according to the invention, optionally further comprising fibrinogen, for use as a hemostat in liver resection surgery wherein administration or medical use of said composition results in a median time to hemostasis (TTH) of about less than 1.9 minutes, such as less than 1.5 minutes, such as about 1.0 minute.

In a further particularly preferred embodiment is provided a sterile powder composition or pharmaceutical composition comprising thrombin according to the invention, optionally further comprising fibrinogen, for use as a hemostat in spinal surgery, wherein administration or medical use of said composition results in a median time to hemostasis (TTH) of about less than 2.0 minutes, such as less than about 1.5 minutes, such as about 1.0 minute.

In a further particularly preferred embodiment is provided a sterile powder composition or pharmaceutical composition comprising thrombin according to the invention, optionally further comprising fibrinogen, for use as a hemostat in soft tissue surgery, wherein administration or medical use of said composition results in a median time to hemostasis (TTH) of about less than 2.5 minutes, such as less than about 2.0 minutes, such as about 1.5 minutes,

In a further particularly preferred embodiment is provided a sterile powder composition or pharmaceutical composition comprising thrombin according to the invention, optionally further comprising fibrinogen, for use as a hemostat in vascular surgery, wherein administration or medical use of said composition results in a median time to hemostasis (TTH) of about less than 3.0 minutes, such as less than about 2.5 minutes, such as about 2.0 minutes.

The present invention also provides a unit dosage form, such as a container containing a sterile powder composition of the present invention.

In one embodiment, the present invention is directed to a container containing a sterile powder formulation comprising 0.01% to 30% w/w of fibrinogen optionally in substantially non-crystalline or amorphous form, and/or comprising 0.01% to 30% of thrombin optionally in substantially non-crystalline or amorphous form, and optionally further comprising a pharmaceutically acceptable excipient, optionally wherein the components are substantially in solid solution within the powder, optionally wherein the particles have a median diameter (×50) of between 10 and 500 microns, and/or a mass median aerodynamic diameter (MMAD) of between 10 and 500 microns, and optionally a rugosity of greater than 1.5. Optionally, a third pharmaceutically-active agent may be present, optionally in crystalline or amorphous form.

In one embodiment, the present invention is directed to a container containing a sterile powder formulation or composition according to the invention wherein the amount contained therein constitutes a therapeutically effective amount, such as from about 0.1 g to 100 g, or from about 0.5 g to about 10 g.

Examples of containers include, but are not limited to, capsules, plastic cartridges, glass vials, blisters, or container closure systems made of metal, polymer (e.g., plastic, elastomer), glass, or the like.

The container may be inserted into a delivery device. The container may be of a suitable shape, size, and material to contain the sterile powder formulation and to provide the sterile powder formulation in a usable condition. For example, the vial, capsule or blister may comprise a wall which comprises a material that does not adversely react with the dry powder formulation. In addition, the wall may comprise a material that allows the container to be opened to allow the sterile powder formulation to be administered. In one or more versions, the wall comprises one or more of gelatin, hydroxypropylmethyl-cellulose (HPMC), polyethyleneglycol-compounded HP C, hydroxypropylcellulose, agar, aluminium foil, or the like.

The use of foil-foil blisters or sachets may be suitable where the sterile powder formulation of the present invention is in substantially amorphous form. The selection of appropriate foils is within the purview of a skilled artisan in view of the teachings herein. The nature of the foils utilized may be driven by the moisture permeability of the seal, and the ability of the material to be formed into a blister of the appropriate size and shape. In one embodiment, the powders are loaded into foil-foil blisters or sachets with a fill mass of between 0.05 and 10 grams.

A pharmaceutical composition or a sterile powder composition of the invention may be of particular value where application to a large surface area is required. This includes surgery and repair of traumatic injuries to various organs such as the liver and spleen. A further advantageous application is in skin grafting for burns patients, and specifically where skin epidermal sheets are cultured in vitro and then transferred to the wound site. The use of fibrin sealant in the latter indication may be particularly effective in patients with extensive burns, providing a biocompatible anchorage for skin grafts. It may also be suitable in the treatment of topical ulcers.

In another presentation of the sterile powder composition or pharmaceutical composition of the invention, the powder or composition may be contained within a sachet or pouch of soluble material. When the pouch is placed onto moist tissue, such as at a wound or surgical site, the material of the pouch dissolves to release the powder from within the pouch.

In an aspect of the present invention, the sterile powder composition or pharmaceutical composition comprising thrombin, optionally further comprising fibrinogen, which is adapted to form a protective or preventative covering or bandage for minor abrasions, cuts, scrapes, scratches, burns, sunburns, ulcers and other skin injuries and irritations, such as bleeding during and post-surgery, and uncontrolled internal and external hemorrhage from heavy trauma and/or battlefield wounds.

In another embodiment of the present invention is provided a sterile powder composition or pharmaceutical composition comprising thrombin, optionally further comprising fibrinogen, for the treatment of mild to moderate bleeding. Mild bleeds are those typically presenting with a blood flow of less than about 5 g/minute whereas moderate bleeds are often about 10 g/minute or less, or about 20 g/minute, or less.

In another embodiment of the present invention is provided a sterile powder composition or pharmaceutical composition comprising thrombin, optionally further comprising fibrinogen, as an aid to surgical hemostasis for mild to moderate bleeding from small vessels when control of bleeding by standard surgical techniques is ineffective or impractical.

In yet another embodiment of the present invention is provided a sterile powder composition or pharmaceutical composition comprising thrombin, optionally further comprising fibrinogen, as an adjunct to hemostasis in patients undergoing open surgical procedures when control of mild or moderate bleeding by conventional surgical techniques including suture, ligature and cautery is ineffective or impractical and as suture support for haemostasis in vascular surgery, optionally wherein the patient is an adult over 18 years of age.

Another embodiment of the present invention provides a sterile powder composition or pharmaceutical composition comprising thrombin, optionally further comprising fibrinogen, is adapted to form a seal on non-superficial tissues or to close open tissues exceeding minor abrasions, cuts, scrapes, scratches, burns, sunburns, ulcers and other skin injuries and irritations. Treatable wounds include: topical wounds; deeper wounds; surgical incisions; severe wounds; battlefield wounds and trauma; and emergency room excessive bleeding, among others. Accordingly, the various applications of the wound sealants include first aid and triage applications for surgical and medical procedures.

A sterile powder composition or pharmaceutical composition comprising thrombin according to the invention, optionally further comprising fibrinogen, may be applied directly to wounds, sutures, incisions and other openings where bleeding may occur. A wound includes damage to any tissue in a living organism. A biological tissue includes connective tissues, endothelial tissues, nervous tissues, muscle tissue and organs. Biological tissues may be selected from the group consisting of bone, skin, cartilage, spleen, muscle, lymphatic, renal, hepatic, blood vessels, lung, dura, bowel and digestive tissue. The tissue may be an internal (e.g. organ) or external tissue (e.g. eye, skin, etc.), and may be a hard tissue (e.g. bone) or a soft tissue (e.g., liver, spleen etc.). The wound may have been caused by any agent, including infection, surgical intervention, burn or trauma. Trauma is defined as an injury caused by a physical force; examples include the consequences of motor vehicle accidents, gunshots and burns.

In another aspect of the invention, a sterile powder composition or pharmaceutical composition comprising thrombin according to the invention, optionally further comprising fibrinogen, may be used for surgical interventions such as in the gastrointestinal system, e.g. the oesophagus, stomach, small intestine, large intestine, bowel, rectum, on parenchymal organs such as the liver, pancreas, spleen, lungs, kidney, adrenal glands, lymph and thyroid glands; surgical interventions in the ear, nose and throat area (ENT) including dental surgery, epistaxis, cardiovascular surgery, such as carotid endarterectomy, femoropopliteal bypass or coronary artery bypass grafting (CABG); aesthetic surgery, spinal surgery, neurological surgery, such as posterior lumbar interbody fusion, microdiscectomy or craniotomy; lymphatic, biliary, and cerebrospinal (CSF) fistulae, air leakages during thoracic and pulmonary surgery, thoracic surgery including surgery of the trachea, bronchi and lungs orthopaedic surgery, such as knee or hip replacement; gynaecological surgical procedures such as caesarian section, hysterectomy, fibroid surgery; vascular surgery, such as shunts; urological, bone (e.g. spongiosa resection), and emergency surgery.

Surgical interventions may include orthopaedic surgery, liver resection, and vascular surgery. The sterile powder composition or pharmaceutical composition comprising thrombin according to the invention, optionally further comprising fibrinogen, demonstrate advantages over existing liquid systems comprising fibrinogen and thrombin, whereby these prior art products require reconstitution, have a short shelf life once reconstituted, often have to be delivered using a double-barreled syringe due to their incompatibility in the liquid state. Furthermore, such liquid presentations are difficult to apply to awkward or complex surfaces of wounds and or organs and are also prone to run off and pool in cavities. The sterile powder composition and formulations described herein, overcome these drawbacks.

In a further aspect of the invention, the sterile powder composition or pharmaceutical composition comprising thrombin according to the invention, optionally further comprising fibrinogen, is administered during or after surgery. The compositions of the invention may be administered to the wound or wounds of a subject, including human, mammal and other veterinary applications.

In a further aspect of the invention, the sterile powder composition or pharmaceutical composition comprising thrombin according to the invention, optionally further comprising fibrinogen, is formulated as a sterile preparation for single-delivery application to a wound site, or as a multi-use preparation. The preparation can be packaged and supplied in formats or formulations including: dry powder, dry adhesive coating, aerosol, dry aerosol, pump spray, medical compress; film; coated plaster; medicated sponge or surgical patch (cf. Tachosil®); hemostatic fleece (cf. Tachocomb®); gauze; salve, semi-gel, gel, foam, paste, suspension, ointment, emulsion, moldable form, nasal plug, surgical dressing, wound packing, bandage, swab, catheter, fiber optic, syringe, pessary, suppository, or suspension in a liquid (non-aqueous) and the like. The formulations may be applied topically to a wound site. Alternatively or in addition, the formulation can be introduced internally into the wound site in the case of, for example, deeper lacerations, arterial wounds, or during surgical procedures.

Another aspect of the invention provides a liquid hemostatic composition comprising the sterile powder composition or pharmaceutical composition comprising thrombin according to the invention, optionally further comprising fibrinogen, formulated as a suspension, for topical delivery on minor abrasions, cuts, scrapes, scratches, burns, sunburns, ulcers, internal venous bleeding, external venous bleeding, and surgical trauma.

Another embodiment of the invention provides a sterile powder composition or pharmaceutical composition comprising thrombin according to the invention, optionally further comprising fibrinogen, as a hemostatic composition for topical delivery on minor abrasions, cuts, scrapes, scratches, burns, sunburns, ulcers, internal venous bleeding, external venous bleeding, and surgical trauma, with said composition comprising the fibrin sealant powder composition in a nonaqueous liquid carrier for forming a thin-film barrier over the site of injury. The formulation may be easily applied to the wound site in variable quantities and will quickly stop bleeding.

Another embodiment of the invention provides a sterile powder composition or pharmaceutical composition comprising thrombin according to the invention, optionally further comprising fibrinogen, for use as a hemostat, wherein administration or medical use of said composition results in a time to hemostasis (TTH) of less than about 10 minutes, or less than about 8 minutes, or less than about 5 minutes, or less than about 4 minutes, or less than about 3 minutes, or less than about 2 minutes, or less.

In another embodiment of the invention provides a sterile powder composition or pharmaceutical composition comprising thrombin according to the invention, further comprising fibrinogen, for use as a hemostat in conjunction with a gelatin sponge or the like, wherein administration or medical use of said composition results in a time to hemostasis (TTH) of less than about 10 minutes, or less than about 8 minutes, or less than about 5 minutes, or less than about 4 minutes, or less than about 3 minutes, or less than about 2 minutes, or less.

In another embodiment of the invention is provided a sterile powder composition or pharmaceutical composition comprising thrombin according to the invention, optionally further comprising fibrinogen, for use as a hemostat, wherein administration or medical use of said composition, optionally in conjunction with a gelatin sponge or the like, results in a median time to hemostasis (TTH) of less than about 10 minutes, or less than about 8 minutes, or less than about 5 minutes, or less than about 4 minutes, or less than about 3 minutes, or less than about 2 minutes, or less, such as about 1.9 minutes, or about 1.5 minutes, or about 1.0 minute. The median TTH value may be obtained from a linear regression analysis of the cumulative distribution data of measured time to hemostasis in any particular bleeding scenario in a number of subjects. According to such a description, fifty percent of the subjects will have a time to hemostasis less than the median TTH, and fifty percent of the subjects will have a time to hemostasis greater than the median TTH. The median TTH in this embodiment is defined as the median time taken to achieve hemostasis in a sample population of subjects, such as where there are at least 10, 20, 30, 40, 50, 75, 100, 125, 150, 200, 300, 400, 500 or more subjects.

The medical use may be any of liver resection surgery, spinal surgery, soft tissue surgery or vascular surgery.

The sterile powder composition or pharmaceutical composition comprising thrombin may be applied using the powder delivery device of co-pending application PCT/GB2009/051714, herein incorporated by reference, for use in surgical interventions such as such as in the gastrointestinal system, e.g. the oesophagus, stomach, small intestine, large intestine, bowel, rectum, on parenchymal organs such as the liver, pancreas, spleen, lungs, kidney, adrenal glands, lymph and thyroid glands; surgical interventions in the ear, nose and throat area (ENT) including dental surgery, epistaxis, cardiovascular surgery, such as carotid endarterectomy, femoropopliteal bypass or coronary artery bypass grafting (CABG); aesthetic surgery, spinal surgery, neurological surgery, such as posterior lumbar interbody fusion, microdiscectomy or craniotomy; lymphatic, biliary, and cerebrospinal (CSF) fistulae, air leakages during thoracic and pulmonary surgery, thoracic surgery including surgery of the trachea, bronchi and lungs orthopaedic surgery, such as knee or hip replacement; gynaecological surgical procedures such as caesarian section, hysterectomy, fibroid surgery; vascular surgery, such as shunts, urological, bone (e.g. spongiosa resection), and emergency surgery.

Surgical interventions may suitably include orthopaedic surgery, liver resection, soft tissue surgery and vascular surgery.

In a particularly preferred embodiment is provided a sterile powder composition or pharmaceutical composition comprising thrombin according to the invention, optionally further comprising fibrinogen, for use as a hemostat in liver resection surgery, spinal surgery, soft tissue surgery or vascular surgery, wherein administration or medical use of said composition results in a median time to hemostasis (TTH) of less than about 2 minutes, or less, such as about 1.9 minutes, or about 1.5 minutes, or about 1.0 minute.

In a further particularly preferred embodiment is provided a sterile powder composition or pharmaceutical composition comprising thrombin according to the invention, optionally further comprising fibrinogen, for use as a hemostat in liver resection surgery or spinal surgery, wherein administration or medical use of said composition results in a median time to hemostasis (TTH) of less than about 1.5 minutes, or about 1.0 minute, or less.

In a particularly preferred embodiment is provided a sterile powder composition or pharmaceutical composition comprising thrombin according to the invention, optionally further comprising fibrinogen, for use as a hemostat in liver resection surgery, spinal surgery, soft tissue surgery or vascular surgery, wherein administration or medical use of said composition results in hemostasis at 3 minutes post administration or medical use in at least 70 or 75% of subjects and/or hemostasis at 5 minutes post administration or medical use in at least 80 or 85 or 90% or more of subjects.

In a further particularly preferred embodiment is provided a sterile powder composition or pharmaceutical composition comprising thrombin according to the invention, optionally further comprising fibrinogen, for use as a hemostat in liver resection surgery or spinal surgery, wherein administration or medical use of said composition results in hemostasis at 3 minutes post administration or medical use in at least 80 or 85 or 90% of subjects and/or hemostasis at 5 minutes post administration or medical use in at least 90 or 95 or 98% or more of subjects.

In a particularly preferred embodiment is provided a sterile powder composition or pharmaceutical composition comprising thrombin according to the invention, optionally further comprising fibrinogen, for use as a hemostat in liver resection surgery, wherein administration or medical use of said composition results in hemostasis at 3 minutes post administration or medical use in at least 70 or 75% or more of subjects, such as about 92, 93, or 94% or more of patients, and/or hemostasis at 5 minutes post administration or medical use in at least 80 or 85 or 90% or more of subjects, such as about 95, 96, 97, 98% or more of subjects.

In a further particularly preferred embodiment is provided a sterile powder composition or pharmaceutical composition comprising thrombin according to the invention, optionally further comprising fibrinogen, for use as a hemostat in spinal surgery, wherein administration or medical use of said composition results in hemostasis at 3 minutes post administration or medical use in at least 70 or 75% or more of subjects, such as about 92, 93, 94, 95 or 96% or more of subjects, and/or hemostasis at 5 minutes post administration or medical use in at least 90 or 95 or 98% or more of subjects, such as about 95, 96, 97, 98% or more of subjects.

In a further particularly preferred embodiment is provided a sterile powder composition or pharmaceutical composition comprising thrombin according to the invention, optionally further comprising fibrinogen, for use as a hemostat in vascular surgery, wherein administration or medical use of said composition results in hemostasis at 3 minutes post administration or medical use in at least 70% or about 75% or more of subjects, such as about 71, 72, 73, 74% or more of subjects, and/or hemostasis at 5 minutes post administration or medical use in at least 80% or 85% or more of subjects, such as about 85, 86, 87% or more of subjects.

In a further particularly preferred embodiment is provided a sterile powder composition or pharmaceutical composition comprising thrombin according to the invention, optionally further comprising fibrinogen, for use as a hemostat in soft tissue surgery, wherein administration or medical use of said composition results in hemostasis at 3 minutes post administration or medical use in at least 70 or 75% or more of subjects, such as about 92, 93, 94% or more of subjects, and/or hemostasis at 5 minutes post administration or medical use in at least 90 or 95 or 98% or more of subjects, such as about 95, 96, 97, 98% or more of subjects.

In a further embodiment of the invention is provided a method of treating a wound or the use of a sterile powder composition comprising thrombin, optionally further comprising fibrinogen, as a hemostat in liver resection surgery, spinal surgery, soft tissue surgery or vascular surgery, optionally wherein administration or medical use of said composition is in conjunction with, for example, a gelatin sponge, gauze or collagen material by first applying the composition to a hemorrhaging site and placing the gelatin sponge, gauze or collagen on top of the composition and wherein the method further optionally comprises the application of moderate manual pressure for not less than about 30 seconds, or not less than about 60 seconds, or not less than about 2 minutes, or not less than about 3 minutes, or not less than about 5 minutes, or not less than about 7 minutes, or not less than about 10 minutes, or longer.

In a further preferred embodiment of the invention is provided a method of treating a wound or the use of a sterile powder composition comprising thrombin, optionally further comprising fibrinogen, as a hemostat in liver resection surgery, spinal surgery, soft tissue surgery or vascular surgery, optionally wherein administration or medical use of said composition is in conjunction with, for example, a gelatin sponge, gauze or collagen material by first applying the composition to a hemorrhaging site and placing the gelatin sponge, gauze or collagen on top of the composition and wherein the method further optionally comprises the application of moderate manual pressure for not less than about 30 seconds, or not less than about 60 seconds, or not less than about 2 minutes, or not less than about 3 minutes, or not less than about 5 minutes, or not less than about 7 minutes, or not less than about 10 minutes, or longer, optionally wherein said administration or medical use of said composition results in a median time to hemostasis (TTH) of less than about 2 minutes, or less, such as about 1.9 minutes, or about 1.5 minutes, or about 1.0 minute.

In a further embodiment of the invention is provided a method of treating a wound or the use of a sterile powder composition comprising thrombin, wherein said sterile powder composition comprises; (i) a composite particle comprising thrombin and fibrinogen, and/or (ii) a mixture of particles that comprise fibrinogen in the absence of thrombin, and particles that comprises thrombin in the absence of fibrinogen, as a hemostat in liver resection surgery, spinal surgery, soft tissue surgery or vascular surgery, wherein administration or medical use of said composition results in a median time to hemostasis (TTH) of less than about 2 minutes, or less, such as about 1.9 minutes, or about 1.5 minutes, or about 1.0 minute.

In another particularly preferred embodiment of the invention is a thrombin powder composition or pharmaceutical composition, optionally further comprising fibrinogen, prepared by removal of liquid from a solution or suspension of said thrombin or fibrinogen, wherein removing said liquid is carried out under aseptic conditions, for use in the treatment of bleeding associated with liver resection surgery, soft tissue surgery, vascular surgery or spinal surgery.

In another particularly preferred embodiment of the invention is a thrombin powder composition or pharmaceutical composition, optionally further comprising fibrinogen, prepared by removal of liquid from a solution or suspension of said thrombin or fibrinogen, wherein removing said liquid is carried out under aseptic conditions, optionally by spray drying, co-spray drying or fluid bed drying, for use in the treatment of bleeding associated with liver resection surgery, soft tissue surgery, vascular surgery or spinal surgery.

In another particularly preferred embodiment of the invention is a thrombin powder composition or pharmaceutical composition, optionally further comprising fibrinogen, prepared by removal of water from an aqueous solution of said thrombin or fibrinogen, wherein removing water is carried out under aseptic conditions, optionally by spray drying, co-spray drying or fluid bed drying, for use in the treatment of bleeding associated with liver resection surgery, soft tissue surgery, vascular surgery or spinal surgery.

In another embodiment of the invention is a method for preparing a sterile powder composition comprising fibrinogen and thrombin prepared by removal of liquid from a solution or suspension of said thrombin or fibrinogen, wherein the powder is produced by a process selected from aseptic spray drying or aseptic fluid bed drying, optionally further comprising a carrier material onto which one or both of the fibrinogen or thrombin is coated onto, and wherein the powder exhibits at least 80% of the thrombin potency or activity of the liquid feedstock, optionally wherein the composition comprises:

(i) a composite particle comprising thrombin and fibrinogen, or

(ii) a mixture of particles that comprise fibrinogen in the absence of thrombin, and particles that comprises thrombin in the absence of fibrinogen, optionally wherein (i) or (ii) are in the form of nanoparticles, nanofibres, fibres, particles, granules, beads, microbeads, microspheres, microcapsules or microparticles, preferably microparticles, and optionally wherein the composition is packaged as a sterile final pharmaceutical product for medical use, such as for direct topical application as a dry powder.

In a further embodiment of the invention there is provided a sterile powder or granulate composition suitable for medical use comprising thrombin and fibrinogen, produced in an evacuatable container with or without a support medium as a receiver, and wherein said composition is packaged as a sterile final pharmaceutical product for medical use. Appropriate support media include sugar and sugar alcohols such as sucrose, trehalose, lactose or mannitol. In this embodiment, it is especially preferred to use the sterile thrombin powder as a support medium, produced from a liquid feedstock, wherein the feedstock comprises a solution or a suspension of thrombin, preferably a solution, wherein the powder is produced by removal of liquid by aseptic spray drying or aseptic fluid bed drying. The aqueous solution or suspension of fibrinogen, optionally further comprising trehalose, is then sprayed aseptically onto said support medium to form a granulate and optionally further packaged aseptically.

Alternatively, the sterile fibrinogen powder may be used as a support medium, wherein the sterile fibrinogen powder is produced by removal of liquid from a feedstock, wherein the feedstock comprises a solution or a suspension of fibrinogen, preferably a solution, by aseptic spray drying or fluid bed drying. A fine thrombin suspension in an organic solvent is sprayed onto said fibrinogen granulate. The aqueous solution or suspension of thrombin, optionally further comprising trehalose and/or a source calcium ions, is then sprayed aseptically onto said support medium to form a granulate and optionally further packaged aseptically.

The sterile granulate can also be obtained by spray-drying fibrinogen concentrate from an aqueous solution onto a receiver, such as mannitol. To that end, a sterile fibrinogen/mannitol-granulate is aseptically obtained first, and then thrombin/calcium salt, for example from an isopropanolic suspension, is sprayed aseptically onto said granulate. The organic solvent prevents the formation of fibrin following the contact between fibrinogen and the thrombin. In addition to fibrinogen, said granulate can also contain other proteins, carbohydrates, amino acids and physiologically safe inorganic salts, and also calcium salt as well. The median particle size (×50) of said sterile granulate is more than about 50 μm and up to about 1000 μm, with the preferred median particle size being between 100 and 200 μm.

The invention also relates to a medical device comprising a composition of the invention.

In another embodiment of the invention is provided process for preparing a viscous, water soluble fibrin sealant paste, salve, ointment or suspension composition comprising the steps of: admixing said sterile powder composition or pharmaceutical composition comprising thrombin according to the invention, optionally further comprising fibrinogen, and a base such as polyethylene glycol having a molecular weight range of from about 200 to 6000. Blends of various molecular weights of PEG may be used. Optionally, the PEG may be a blend having an average molecular weight of in the range of about 500 to 1,000, as a 1:1, or 1:2 or 1:3 or 1:5 or 1:9 blend of PEG 300 MW and 1500 MW. Use of lower grades of PEG will produce lighter, less viscous suspensions which can be packaged and delivered via a pump spray. Such suspensions may optionally include a surfactant or other suitable suspending agent, to prevent flocculation. Preparation and the formulation of such formats are known to those skilled in the art.

The paste, salve, ointment or suspension composition may also be used in conjunction with, for example, a gelatin sponge, gauze or collagen material by either coating such material as a substrate with the composition and applying it to the hemorrhaging site or first applying the composition to a hemorrhaging site and placing the gelatin sponge, gauze or collagen on top of the composition and applying pressure thereto. The ointment, salve or paste of the present invention has a viscosity and potency which is high enough to permit its hemostatic effective use by a surgeon by dipping of a gloved finger into the paste and placing the paste over the bleeding site. The polyethylene glycol may have an average molecular weight range of from about 500 to 1000 or about 900. Grades of polyethylene glycol can be combined with one another to produce unique properties. For example, PEG 1500, a solid at room temperature, while not soluble in liquid PEG 300 at room temperature may be combined together and heated above the melting point of the higher melting glycol (i.e. PEG 1500) to form a solution. For example, PEG 300 which is a liquid, is mixed with an equal weight of PEG 1500, a solid melting at 43 degrees C., and the two heated together at or above the melting point of PEG 1500 such that they liquefy to a substantially homogeneous solution, and when that solution is cooled to room temperature, it forms a smooth, soft paste. This paste may be water soluble, and sufficiently yielding to spread readily on tissue or skin.

The invention further comprises a method for reducing bleeding at a hemorrhaging site by applying a paste composition comprising a hemostatic effective amount of the sterile powder composition or pharmaceutical composition comprising thrombin according to the invention, optionally further comprising fibrinogen, in a base comprising polyethylene glycol to the hemorrhaging site of a subject. The paste may be applied in combination with a fibrous gauze material or by itself in paste form to the hemorrhaging site.

In a further embodiment of the invention, the sterile powder composition or pharmaceutical composition comprising thrombin according to the invention, optionally further comprising fibrinogen, is admixed with a propellant and packaged in an aerosol container, optionally with a polymer such as PVP (see U.S. Pat. No. 4,752,466). This therefore provides a convenient way to deliver dry powdered thrombin directly to a wound, or directly onto a haemostat or support material as described herein. The amount of fibrin sealant powder composition used in each can could differ according to the potency or activity desired, but typically might be on the order of magnitude of 0.5 to 1.0 gram. The propellant, in liquified form, then is filled into the aerosol container through the valve from a tank where it exists in liquified form. The amount of propellant used typically might be on the order of 10 grams. Other methods of filling an aerosol container are well known and may be used if desired. Inside the aerosol container, the synthetic polymer e.g. PVP completely dissolves in the propellant. The fibrin sealant powder composition does not dissolve, but exists in a very finely divided state, i.e., it is suspended in the propellant, where it exists as a finely divided milky suspension. When the valve is depressed to spray the material from the aerosol container, a mixture of fibrin sealant powder composition, propellant and optionally PVP is emitted. The sterile powder composition or pharmaceutical composition comprising thrombin according to the invention, optionally further comprising fibrinogen, comes out as a dry white powder. The propellant evaporates quickly and disappears. Aerosol containers and components thereof designed for dispensing powder sprays are commercially available, and may be used in the present invention. In the “Handbook of Aerosol Technology” by Paul Sanders (Van Nostrand, Reinhold Company, N. Y. 1979, 2nd. ed. Chapter 21 entitled “Aerosol Suspensions) (Powders) gives helpful background information. Propellants may include those of the HFA series.

The aerosol package of the present invention may prepared and handled in such manner that its contents are sterile when sprayed. The use of bacterial filters and aseptic processing techniques results in a sterile product.

The aerosol of the present invention is designed to be stored at room temperature. Suitably in this form it is relatively stable for at least for periods of 6 months, for example, where the sterile powder composition is substantially in a glassy form.

According to Kheirabadi et al. (J. trauma, Injury, Infection and Critical Care; 71: No 1, July Supplement 2011), the ideal hemostatic dressing for tactical applications demonstrates at least one or preferably all of the following characteristics: (a), is approved by the FDA; (b), stops severe arterial and/or venous bleeding in less than or equal to two minutes; (c), has no toxicity or side effects; (d), causes no pain or thermal injury, (e), poses no risk to medics; (f) is ready to use and requires little or no training; (g), is durable and lightweight; (h), is flexible enough to fit complex wounds and is easily removed without leaving residues; (i), is stable and functional at extreme temperatures (−10.degree. C. to +40.degree. C.) for at least two weeks; (j), is practical and easy to use under austere conditions (low visibility, rain, wind, etc.); (k), has a long shelf-life, preferably greater than 2 years; (l), is effective on junctional wounds not amenable without tourniquet; (m), is inexpensive and cost-effective, and; (n) is biodegradable and bioabsorbable. In one aspect the products of the invention suitably meet 1, 2, 3, 4, 5, 6, 7, 8, 9, 10, 11, 12, 13 or all 14 of the above criteria, preferably at least 8, 9, 10, 11, 12, 13 or 14.

In an embodiment of the invention is provided a sterile powder composition or pharmaceutical composition comprising thrombin, optionally further comprising fibrinogen, further comprising an absorbable carrier of a biocompatible, biodegradable polymer, said sterile powder optionally dispersed at least partially through, in or on said absorbable carrier.

In another embodiment of the invention is provided a sterile powder composition or pharmaceutical composition comprising thrombin, optionally further comprising fibrinogen, further comprising an absorbable carrier of a biocompatible, biodegradable polymer, said sterile powder optionally dispersed at least partially through, in or on said absorbable carrier, optionally wherein the powder further comprises a pharmaceutically acceptable excipient or carrier, or amorphous material or polyol, or additive material, as described above.

In a further embodiment of the invention is provided a sterile powder composition or pharmaceutical composition comprising thrombin, optionally further comprising fibrinogen, further comprising an absorbable carrier of a biocompatible, biodegradable polymer, said sterile powder dispersed at least partially through, in or on said absorbable carrier, and said sterile powder is dispersed and/or fixed through, in or on said absorbable carrier.

In another embodiment of the invention is provided a pharmaceutical composition comprising an absorbable carrier of a biocompatible, biodegradable polymer, and dispersed at least partially through, in or on said absorbable carrier, a sterile powder composition comprising thrombin, optionally further comprising fibrinogen, optionally wherein the sterile powder further comprise a pharmaceutically acceptable excipient or carrier, or amorphous material or polyol, or additive material, as described above, and wherein the sterile powder is optionally dispersed and/or fixed homogeneously through, in or on said absorbable carrier.

In another embodiment of the invention is provided a pharmaceutical composition comprising an absorbable carrier of a biocompatible, biodegradable polymer, and dispersed at least partially through, in or on said absorbable carrier, a sterile powder composition comprising thrombin and further comprising fibrinogen, optionally wherein the sterile powder further comprise a pharmaceutically acceptable excipient or carrier, or amorphous material or polyol, or additive material, as described above, wherein fibrinogen is present in an amount of from about 0.1-15 mg/cm.sup.2, or about 0.5 to 5 mg/cm.sup.2, and thrombin is present in an amount of from about 0.01 to 500 IU/cm.sup.2, or about 0.1 to 50 IU/cm.sup.2. Alternatively, for compositions in a form with three-dimensions (such as a pad, foam and the like where content may be expressed in terms of volume), the fibrinogen is present in an amount of from about 0.1-15 mg/cm.sup.3, or about 0.5 to 5 mg/cm.sup.3, and thrombin is present in an amount of from about 0.01 to 500 IU/cm.sup.3, or about 0.1 to 50 IU/cm.sup.3.

In another embodiment of the invention is provided a pharmaceutical composition comprising an absorbable carrier of a biocompatible, biodegradable polymer, and dispersed at least partially through, in or on said absorbable carrier, a sterile powder composition comprising thrombin, optionally further comprising fibrinogen, optionally wherein the sterile powder further comprise a pharmaceutically acceptable excipient or carrier, or amorphous material or polyol, or additive material, as described above, wherein the carrier is flexible and/or porous, and optionally further comprises a plasticizer and/or viscosifying agent.

In another embodiment of the invention is provided a pharmaceutical composition comprising an absorbable carrier of a biocompatible, biodegradable polymer, and dispersed at least partially through, in or on said absorbable carrier, a sterile powder composition comprising thrombin, wherein said sterile powder composition comprises; (i) a composite particle comprising thrombin and fibrinogen, and/or (ii) a mixture of particles that comprise fibrinogen in the absence of thrombin, and particles that comprises thrombin in the absence of fibrinogen, optionally wherein the sterile powder further comprise a pharmaceutically acceptable excipient or carrier, or amorphous material or polyol, or additive material, as described above, and wherein said sterile powder composition constitutes a layer on one or more surfaces of the absorbable carrier.

In another embodiment of the invention is provided a pharmaceutical composition comprising an absorbable carrier of a biocompatible, biodegradable polymer, and dispersed at least partially through, in or on said absorbable carrier, a sterile powder composition comprising thrombin, wherein said sterile powder composition comprises; (i) a composite particle comprising thrombin and fibrinogen, and/or (ii) a mixture of particles that comprise fibrinogen in the absence of thrombin, and particles that comprises thrombin in the absence of fibrinogen, optionally wherein the sterile powder further comprise a pharmaceutically acceptable excipient or carrier, or amorphous material or polyol, or additive material, as described above, and wherein said sterile powder constitute a hemostatic layer within or on one or more surfaces of the absorbable carrier, and optionally wherein the pharmaceutical composition optionally further comprises one or more support layers (e.g. a backing material or an internal support material) and/or release layers.

In another embodiment of the invention is provided a pharmaceutical composition comprising an absorbable carrier of a biocompatible, biodegradable polymer, and optionally dispersed at least partially through, in or on said absorbable carrier, a sterile powder composition comprising thrombin, wherein said sterile powder composition comprises; (i) a composite particle comprising thrombin and fibrinogen, and/or (ii) a mixture of particles that comprise fibrinogen in the absence of thrombin, and particles that comprises thrombin in the absence of fibrinogen, optionally wherein the sterile powder further comprise a pharmaceutically acceptable excipient or carrier, or amorphous material or polyol, or additive material, as described above, optionally formed or cast as a single piece, optionally wherein the powder is homogeneous throughout, optionally wherein the composition further contains a binding agent to facilitate or improve the adherence of the particles to one another and/or to any support layer(s) and/or the absorbable carrier or matrix and/or the tissue.

Illustrative examples of suitable binding agents include, but are not limited to, sucrose, mannitol, sorbitol, gelatin, hyaluron and its derivatives, such as hyaluronic acid, maltose, povidone, starch, chitosan and its derivatives, and cellulose derivatives, such as carboxymethylcellulose, hydroxypropylcellulose, as well as mixtures of two or more thereof.

In another embodiment of the invention is provided a pharmaceutical composition comprising an absorbable carrier of a biocompatible, biodegradable polymer, and optionally dispersed at least partially through, in or on said absorbable carrier, a sterile powder composition comprising; (i) a composite particle comprising thrombin and fibrinogen, and/or (ii) a mixture of particles that comprise fibrinogen in the absence of thrombin, and particles that comprises thrombin in the absence of fibrinogen, optionally wherein the sterile powder further comprise a pharmaceutically acceptable excipient or carrier, or amorphous material or polyol, or additive material, as described above, and optionally further containing one or more suitable fillers, such as sucrose, lactose, maltose, silk, fibrin, collagen, albumin, hyaluronate and its derivatives, such as hyaluronic acid, polysorbates (Tween™), chitin, chitosan and its derivatives, such as NOCC-chitosan, alginic acid and salts thereof, cellulose and derivatives thereof, proteoglycans, glycolic acid polymers, lactic acid polymers, glycolic acid/lactic acid co-polymers, and mixtures of two or more thereof.

In another embodiment of the invention is provided a pharmaceutical composition comprising an absorbable carrier of a biocompatible, biodegradable polymer, and optionally dispersed at least partially through, in or on said absorbable carrier, a sterile powder composition comprising thrombin, wherein said sterile powder composition comprises; (i) a composite particle comprising thrombin and fibrinogen, and/or (ii) a mixture of particles that comprise fibrinogen in the absence of thrombin, and particles that comprises thrombin in the absence of fibrinogen, optionally wherein the sterile powder further comprise a pharmaceutically acceptable excipient or carrier, or amorphous material or polyol, or additive material, as described above, optionally wherein the sterile powder further comprises one or more suitable solubilizing agents, such as sucrose, dextrose, mannose, trehalose, mannitol, sorbitol, albumin, hyaluron and its derivatives, such as hyaluronic acid, polysorbate (Tween™), sorbitan (SPAN™) and mixtures of two or more thereof.

In another embodiment of the invention is provided a pharmaceutical composition comprising an absorbable carrier of a biocompatible, biodegradable polymer, and optionally dispersed at least partially through, in or on said absorbable carrier, a sterile powder composition comprising thrombin, wherein said sterile powder composition comprises; (i) a composite particle comprising thrombin and fibrinogen, and/or (ii) a mixture of particles that comprise fibrinogen in the absence of thrombin, and particles that comprises thrombin in the absence of fibrinogen, optionally wherein the sterile powder further comprise a pharmaceutically acceptable excipient or carrier, or amorphous material or polyol, or additive material, as described above, optionally wherein the sterile powder further is optionally homogeneous throughout, optionally wherein the composition further contains a suitable source of calcium ions, such as calcium chloride, and/or a fibrin cross-linker, such as a transaminase (e.g. Factor XIII/XIIIa) or glutaraldehyde.

In another embodiment of the invention is provided a pharmaceutical composition comprising an absorbable carrier of a biocompatible, biodegradable polymer, and optionally dispersed at least partially through, in or on said absorbable carrier, a sterile powder composition comprising thrombin, wherein said sterile powder composition comprises; (i) a composite particle comprising thrombin and fibrinogen, and/or (ii) a mixture of particles that comprise fibrinogen in the absence of thrombin, and particles that comprises thrombin in the absence of fibrinogen, optionally wherein the sterile powder further comprise a pharmaceutically acceptable excipient or carrier, or amorphous material or polyol, or additive material, as described above, optionally wherein said pharmaceutical composition further comprise one or more support layers. As used herein, a “support layer” refers to a material that sustains or enhances the structural integrity of the composition and/or the fibrin clot formed when such a composition is applied to wound. In certain embodiments of the present invention, the support layer comprises a backing material on the side of the pharmaceutical composition opposite the side to be applied to wounded tissue. Such a backing material may be affixed with a physiologically-acceptable adhesive or may be self-adhesive. The backing material may comprise one or more resorbable materials or one or more non-resorbable materials, or mixtures thereof. Optionally, the backing material is a single resorbable material. Any suitable resorbable material known and available to those skilled in the art may be employed in the present invention. For example, the resorbable material may be a proteinaceous substance, such as silk, fibrin, keratin, collagen and/or gelatin. Alternatively, the resorbable material may be a carbohydrate substance, such as alginates, hyaluronan and its derivatives, such as hyaluronic acid, sodium hyaluronate, chitin, cellulose, proteoglycans (e.g. poly-N-acetyl glucosamine), lactic acid polymers, glycolic acid polymers, or glycolic acid/lactic acid co-polymers. The resorbable material may also comprise a mixture of proteinaceous substances or a mixture of carbohydrate substances or a mixture of both proteinaceous substances and carbohydrate substances. Suitable examples of resorbable materials include, but are not limited to, the materials sold under the trade names DEXON™ (a glycolic acid polymer) and VICRYL™ (a glycolic acid/lactic acid copolymer). Any suitable non-resorbable material known and available to those skilled in the art may be employed as the backing material, examples of which include, but are not limited to, paper and paper products, latex, plastics, cotton, silicone polymers, gauze and the like.

In another embodiment of the invention is provided a pharmaceutical composition comprising an absorbable carrier of a biocompatible, biodegradable polymer, and optionally dispersed at least partially through, in or on said absorbable carrier, a sterile powder composition comprising thrombin, wherein said sterile powder composition comprises; (i) a composite particle comprising thrombin and fibrinogen, and/or (ii) a mixture of particles that comprise fibrinogen in the absence of thrombin, and particles that comprises thrombin in the absence of fibrinogen, optionally wherein the sterile powder further comprise a pharmaceutically acceptable excipient or carrier, or amorphous material or polyol, or additive material, as described above, wherein the pharmaceutical composition may also optionally further comprise a release layer. As used herein, a “release layer” refers to a layer containing one or more agents (“release agents”) which promote or allow removal of the pharmaceutical composition from a mold in which it has been manufactured. A suitable such agent is sucrose, but other suitable release agents include gelatin, mannitol, sorbitol, hyaluron and its derivatives, such as hyaluronic acid, and glucose. Alternatively, such one or more release agents may be contained in the hemostatic layer of microparticles, if presented in such a configuration. The various layers of the inventive compositions may be affixed to one another by any suitable means known and available to those skilled in the art. For example, a physiologically-acceptable adhesive may be applied to a backing material (when present), and the pharmaceutical composition subsequently affixed thereto.

In certain embodiments of the present invention, the physiologically-acceptable adhesive has a shear strength and/or structure such that the backing material can be separated from the fibrin clot formed by or within a pharmaceutical composition according to the invention, after its application to wounded tissue. In other embodiments, the physiologically-acceptable adhesive has a shear strength and/or structure such that the backing material cannot be separated from the composition after said application.

During use of a pharmaceutical composition comprising an absorbable carrier of a biocompatible, biodegradable polymer, and optionally dispersed at least partially through, in or on said absorbable carrier, a sterile powder composition comprising thrombin, wherein said sterile powder composition comprises; (i) a composite particle comprising thrombin and fibrinogen, and/or (ii) a mixture of particles that comprise fibrinogen in the absence of thrombin, and particles that comprises thrombin in the absence of fibrinogen, optionally wherein the sterile powder further comprise a pharmaceutically acceptable excipient or carrier, or amorphous material or polyol, or additive material, as described above, the fibrinogen and the thrombin may be activated at the time of application to the wounded tissue by the endogenous fluids of the patient escaping from the hemorrhaging wound.

In another embodiment of the invention is provided a pharmaceutical composition comprising an absorbable carrier of a biocompatible, biodegradable polymer, and optionally dispersed at least partially through, in or on said absorbable carrier, a sterile powder composition comprising thrombin, wherein said sterile powder composition comprises; (i) a composite particle comprising thrombin and fibrinogen, and/or (ii) a mixture of particles that comprise fibrinogen in the absence of thrombin, and particles that comprises thrombin in the absence of fibrinogen, optionally wherein the sterile powder further comprise a pharmaceutically acceptable excipient or carrier, or amorphous material or polyol, or additive material, as described above, wherein said pharmaceutical composition may also contain one or more pharmaceutically-active agents as described herein such as growth factors, polyclonal and monoclonal antibodies, drugs, and other compounds including, but not limited to, the following: fibrinolysis inhibitors, such as aprotonin, tranexamic acid and epsilon-amino-caproic acid; antibiotics, such as tetracycline and metronidazole, ciprofloxacin and amoxicillin; anticoagulants, such as activated protein C, prostaglandins (particularly (PGI.sub.2), leukotrienes, heparin, ADPase, prostacyclins, antithrombin III, and plasminogen activator; steroids, such as dexamethasone, inhibitors of prostacyclin, prostaglandins, leukotrienes and/or kinins to inhibit inflammation; cardiovascular drugs, such as calcium channel blockers, vasodilators and vasoconstrictors; local anaesthetics such as bupivacaine; and antiproliferative/antitumor drugs such as 5-fluorouracil (5-FU), Taxol® (paclitaxel) and/or Taxotere® (docetaxel); anti-virals, such as gancyclovir, zidovudine, amantidine, trifluridine, acyclovir, vidarabine, ribaravin, dideoxyuridine and antibodies to viral components; mammalian gene products; cytokines, such as alpha- or beta-tumour necrosis factor, alpha- or beta- or gamma-Interferon, and interleukins; colony stimulating factors; erythropoietin; antifungals, such as Diflucan® (fluconazole), ketaconazole and nystatin; antiparasitic gents, such as pentamidine; anti-inflammatory agents, such as alpha-1-anti-trypsin and alpha-1-antichymotrypsin; anaesthetics, such as bupivacaine; analgesics; antiseptics; hormones; vitamins and other nutritional supplements; glycoproteins; fibronectin; peptides and proteins; carbohydrates (both simple and/or complex); proteoglycans; antiangiogenins; antigens; lipids or liposomes; oligonucleotides (sense and/or anti-sense DNA and/or RNA); and gene therapy reagents. In other embodiments of the present invention, the backing layer, if present, may contain one or more bioactives.

In another embodiment of the invention is provided a pharmaceutical composition comprising an absorbable carrier of a biocompatible, biodegradable polymer, and dispersed at least partially through, in or on said absorbable carrier, a sterile powder composition comprising thrombin, wherein said sterile powder composition comprises; (i) a composite particle comprising thrombin and fibrinogen, and/or (ii) a mixture of particles that comprise fibrinogen in the absence of thrombin, and particles that comprises thrombin in the absence of fibrinogen, optionally wherein the sterile powder further comprise a pharmaceutically acceptable excipient or carrier, or amorphous material or polyol, or additive material, as described above, wherein said sterile powder is at least partially distributed through or on said absorbable carrier, and wherein said pharmaceutical composition optionally comprises fibrinogen in an amount of from about 0.1-15 mg/cm.sup.2 and/or comprises thrombin in an amount of from about 0.01 to 500 IU/cm.sup.2, wherein the absorbable carrier optionally comprises chitosan, or derivative or salt or co-polymer thereof; gelatin, collagen or a polyurethane and wherein the absorbable carrier is optionally in the form of a porous matrix.

With reference to the term “partially through”, it is intended that the sterile powder composition comprising thrombin is incorporated into the absorbable carrier at least at a single surface or may be present as a distribution presenting a concentration gradient relative to one of the surfaces of said absorbable carrier, or present as an optionally homogenous distribution throughout said absorbable carrier. In one configuration, the composition according to the invention does not include simple, non-agglomerated binary or tertiary blends of sterile powders or microparticles containing fibrinogen and/or sterile powders or microparticles containing thrombin and/or pharmaceutically acceptable excipient or carrier, or amorphous material or polyol, or additive material, such as those described in WO 2010/136588.

In another embodiment of the invention is provided a pharmaceutical composition comprising an absorbable carrier of a biocompatible, biodegradable polymer, and optionally dispersed at least partially through, in or on said absorbable carrier, a sterile powder composition comprising thrombin, wherein said sterile powder composition comprises; (i) a composite particle comprising thrombin and fibrinogen, and/or (ii) a mixture of particles that comprise fibrinogen in the absence of thrombin, and particles that comprises thrombin in the absence of fibrinogen, optionally wherein the sterile powder further comprise a pharmaceutically acceptable excipient or carrier, or amorphous material or polyol, or additive material as described above, wherein said sterile powder is at least partially distributed through or on said absorbable carrier, and wherein said pharmaceutical composition optionally comprises fibrinogen in an amount of from about 0.1-15 mg/cm.sup.2 or about 0.5 to 5 mg/cm.sup.2, and/or thrombin in an amount of from about 0.01 to 500 IU/cm.sup.2, or about 0.1 to 50 IU/cm.sup.2, wherein the absorbable carrier comprises chitosan, or derivative or salt or co-polymer thereof; gelatin, collagen or a polyurethane and wherein the absorbable carrier is optionally in the form of a porous matrix.

In another embodiment of the invention is provided a pharmaceutical composition comprising an absorbable carrier of a biocompatible, biodegradable polymer, and optionally dispersed at least partially through, in or on said absorbable carrier, a sterile powder composition comprising thrombin, wherein said sterile powder composition comprises; (i) a composite particle comprising thrombin and fibrinogen, and/or (ii) a mixture of particles that comprise fibrinogen in the absence of thrombin, and particles that comprises thrombin in the absence of fibrinogen, optionally wherein the sterile powder further comprises a pharmaceutically acceptable excipient or carrier, or amorphous material or polyol, or additive material as described above, wherein said sterile powder is dispersed and/or fixed, at least partially through or on said absorbable carrier, and wherein said pharmaceutical composition optionally comprises fibrinogen in an amount of from about 0.1-15 mg/cm.sup.2 or about 0.5 to 5 mg/cm.sup.2, and/or thrombin in an amount of from about 0.01 to 500 IU/cm.sup.2, or about 0.1 to 50 IU/cm.sup.2, wherein the absorbable carrier comprises chitosan, or derivative or salt or co-polymer thereof; gelatin, collagen or a polyurethane and wherein the absorbable carrier is optionally in the form of a porous matrix, optionally wherein the composition, has a porosity or void fraction of between 1 and 99.9%, or about between 5 and 99%, or about between 10 and 98%, or about between 15 and 95%, wherein the porosity or void fraction is the fraction of the volume of voids over the total volume, expressed as a percentage. Alternatively, pores when present in the composition may have a diameter of from about 0.5 microns to about 5 mm, or from about 1 micron to about 1 mm or even from about 10 microns to about 500 microns.

In another embodiment of the invention is provided a pharmaceutical composition comprising an absorbable carrier of a biocompatible, biodegradable polymer, and dispersed at least partially through, in or on said absorbable carrier, a sterile powder composition comprising thrombin, wherein said sterile powder composition comprises; (i) a composite particle comprising thrombin and fibrinogen, and/or (ii) a mixture of particles that comprise fibrinogen in the absence of thrombin, and particles that comprises thrombin in the absence of fibrinogen, optionally wherein the sterile powder further comprise a pharmaceutically acceptable excipient or carrier, or amorphous material or polyol, or additive material as described above, wherein said sterile powder is dispersed and/or fixed, at least partially through or on said absorbable carrier, and wherein said pharmaceutical composition comprises fibrinogen in an amount of from about 0.1-15 mg/cm.sup.2 or about 0.5 to 5 mg/cm.sup.2, and/or thrombin in an amount of from about 0.01 to 500 IU/cm.sup.2, or about 0.1 to 50 IU/cm.sup.2, wherein the absorbable carrier comprises chitosan, or derivative or salt or co-polymer thereof; gelatin, collagen or a polyurethane and wherein the absorbable carrier is optionally in the form of a porous and flexible matrix which has at least one of the following physical properties: an elasticity module in the range of from about 5 to about 100 N/cm, such as from about 10 to 50 N/cm; and a density of from about 0.1 to 50 mg/cm.sup.3, such as from about 1 to 10 mg/cm.sup.3.

In another embodiment of the present invention is provided a composition for hemostasis, tissue sealing and tissue gluing comprising an absorbable carrier of a biocompatible, biodegradable polymer, and optionally dispersed at least partially through, in or on said absorbable carrier, a sterile powder composition comprising thrombin, wherein said sterile powder composition comprises; (i) a composite particle comprising thrombin and fibrinogen, and/or (ii) a mixture of particles that comprise fibrinogen in the absence of thrombin, and particles that comprises thrombin in the absence of fibrinogen, optionally wherein the sterile powder further comprises a pharmaceutically acceptable excipient or carrier, or amorphous material or polyol, or additive material as described above, wherein said sterile powder is dispersed and/or fixed, at least partially through or on said absorbable carrier, and wherein said pharmaceutical composition or sterile powder comprises fibrinogen in an amount of from about 0.1-15 mg/cm.sup.2 or about 0.5 to 5 mg/cm.sup.2, and/or thrombin in an amount of from about 0.01 to 500 IU/cm.sup.2, or about 0.1 to 50 IU/cm.sup.2, and wherein the absorbable carrier is optionally in the form of a porous and/or flexible matrix, wherein the absorbable carrier has at least one of the following physical properties: an elasticity module in the range of from about 5 to about 100 N/cm, such as from about 10 to 50 N/cm; and a density of from about 0.1 to 50 mg/cm.sup.3, such as from about 1 to 10 mg/cm.sup.3

In another embodiment of the invention is provided a pharmaceutical composition comprising an absorbable carrier of a biocompatible, biodegradable polymer, and optionally dispersed at least partially through, in or on said absorbable carrier, a sterile powder composition comprising thrombin, wherein said sterile powder composition comprises; (i) a composite particle comprising thrombin and fibrinogen, and/or (ii) a mixture of particles that comprise fibrinogen in the absence of thrombin, and particles that comprises thrombin in the absence of fibrinogen, optionally wherein the sterile powder further comprises a pharmaceutically acceptable excipient or carrier, or amorphous material or polyol, or additive material as described above, wherein said sterile powder is dispersed and/or fixed, at least partially through or on said absorbable carrier, and wherein said sterile powder or pharmaceutical composition comprises fibrinogen in an amount of from about 0.1-15 mg/cm.sup.2 or about 0.5 to 5 mg/cm.sup.2, and/or thrombin in an amount of from about 0.01 to 500 IU/cm.sup.2, or about 0.1 to 50 IU/cm.sup.2, optionally wherein the absorbable carrier is in the form of a porous matrix, and wherein the composition is optionally in the form of a single, discrete, solid unit having a three-dimensional structure.

In another embodiment of the invention is provided a pharmaceutical composition comprising an absorbable carrier of a biocompatible, biodegradable polymer, and optionally dispersed at least partially through, in or on said absorbable carrier, a sterile powder composition comprising thrombin, wherein said sterile powder composition comprises; (i) a composite particle comprising thrombin and fibrinogen, and/or (ii) a mixture of particles that comprise fibrinogen in the absence of thrombin, and particles that comprises thrombin in the absence of fibrinogen, optionally wherein the sterile powder further comprise a pharmaceutically acceptable excipient or carrier, or amorphous material or polyol, or additive material as described above, wherein said sterile powder is dispersed and/or fixed, at least partially through or on said absorbable carrier, and wherein said sterile powder or pharmaceutical composition comprises fibrinogen in an amount of from about 0.1-15 mg/cm.sup.2 or about 0.5 to 5 mg/cm.sup.2, and/or thrombin in an amount of from about 0.01 to 500 IU/cm.sup.2, or about 0.1 to 50 IU/cm.sup.2, wherein said pharmaceutical composition is optionally provided as a dry adhesive coating, aerosol, dry aerosol, pump spray, medical compress; film; coated plaster; medicated sponge or surgical patch, hemostatic fleece; hemostatic pad; gauze; salve, semi-gel, gel, foam, paste, suspension, ointment, emulsion, moldable form, nasal plug, surgical dressing, wound packing, bandage, swab, catheter, fibre optic, syringe, pessary, suppository, or suspension in a liquid or non-aqueous liquid.

In another embodiment of the invention is provided a pharmaceutical composition comprising an absorbable carrier of a biocompatible, biodegradable polymer, and optionally dispersed at least partially through, in or on said absorbable carrier, a sterile powder composition comprising thrombin, wherein said sterile powder composition comprises; (i) a composite particle comprising thrombin and fibrinogen, and/or (ii) a mixture of particles that comprise fibrinogen in the absence of thrombin, and particles that comprises thrombin in the absence of fibrinogen, optionally wherein the sterile powder further comprises a pharmaceutically acceptable excipient or carrier, or amorphous material or polyol, or additive material as described above, wherein said sterile powder is dispersed and/or fixed, at least partially through or on said absorbable carrier, and wherein said sterile powder or pharmaceutical composition comprises fibrinogen in an amount of from about 0.1-15 mg/cm.sup.2 or about 0.5 to 5 mg/cm.sup.2, and/or thrombin in an amount of from about 0.01 to 500 IU/cm.sup.2, or about 0.1 to 50 IU/cm.sup.2, wherein said pharmaceutical composition is provided as a single, discrete, solid unit having a three-dimensional structure, selected from a medical compress; film; coated plaster; medicated sponge, surgical patch, hemostatic fleece; hemostatic pad; gauze; moldable form, nasal plug, surgical dressing, wound packing, bandage, swab, catheter, fibre optic, syringe, pessary, or suppository.

In another embodiment of the invention, is provided a pharmaceutical composition comprising an absorbable carrier of a biocompatible, biodegradable polymer, and optionally dispersed at least partially through, in or on said absorbable carrier, a sterile powder composition comprising thrombin, wherein said sterile powder composition comprises; (i) a composite particle comprising thrombin and fibrinogen, and/or (ii) a mixture of particles that comprise fibrinogen in the absence of thrombin, and particles that comprises thrombin in the absence of fibrinogen, optionally wherein the sterile powder further comprises a pharmaceutically acceptable excipient or carrier, or amorphous material or polyol, or additive material as described above, wherein said sterile powder is dispersed and/or fixed, at least partially through or on said absorbable carrier, and wherein said sterile powder or pharmaceutical composition optionally comprises fibrinogen in an amount of from about 0.1-15 mg/cm.sup.2 or about 0.5 to 5 mg/cm.sup.2, and/or thrombin in an amount of from about 0.01 to 500 IU/cm.sup.2, or about 0.1 to 50 IU/cm.sup.2, wherein said pharmaceutical composition can be used as a topical hemostat to stop bleeding. In the present context, the time it takes to stop bleeding is called the time to hemostasis (TTH). If a pressure sheet is used, measurement of TTH typically starts when a pressure sheet is applied to the bleeding site, pressure subsequently being applied, and runs until bleeding has stopped, by visualization of the dressing and/or an indication of bleeding through or around the dressing, is not observed.

In another embodiment of the invention is provided a method for treating wounded tissue in a patient, comprising applying a pharmaceutical composition comprising an absorbable carrier of a biocompatible, biodegradable polymer, and optionally dispersed at least partially through, in or on said absorbable carrier, a sterile powder composition comprising thrombin, wherein said sterile powder composition comprises; (i) a composite particle comprising thrombin and fibrinogen, and/or (ii) a mixture of particles that comprise fibrinogen in the absence of thrombin, and particles that comprises thrombin in the absence of fibrinogen, optionally wherein the sterile powder further comprise a pharmaceutically acceptable excipient or carrier, or amorphous material or polyol, or additive material, as described above, wherein said composition is applied to wounded tissue, and suitably applying sufficient pressure to the composition for a sufficient time for enough fibrin to form to reduce the loss of blood and/or other fluid from the wound. For example, the duration of pressure application may be from about 30 seconds to about 10 minutes.

In a further embodiment of the invention is provided a method of treating a wound or the use of a pharmaceutical composition comprising an absorbable carrier of a biocompatible, biodegradable polymer, and optionally dispersed at least partially through, in or on said absorbable carrier, a sterile powder composition comprising thrombin, wherein said sterile powder composition comprises; (i) a composite particle comprising thrombin and fibrinogen, and/or (ii) a mixture of particles that comprise fibrinogen in the absence of thrombin, and particles that comprises thrombin in the absence of fibrinogen, optionally wherein the sterile powder further comprise a pharmaceutically acceptable excipient or carrier, or amorphous material or polyol, or additive material as described above, wherein said sterile powder is dispersed and/or fixed, at least partially through or on said absorbable carrier, and wherein said sterile powder or pharmaceutical composition comprises fibrinogen in an amount of from about 0.1-15 mg/cm.sup.2 or about 0.5 to 5 mg/cm.sup.2, and/or thrombin in an amount of from about 0.01 to 500 IU/cm.sup.2, or about 0.1 to 50 IU/cm.sup.2, as a hemostat, wherein application of the pharmaceutical composition to a wound results in a TTH of about 10 minutes or less, about 5 minutes or less, or about 3 minutes or less.

In a further embodiment of the invention is provided a method of treating a wound or reducing bleeding at a haemorrhaging site, or the use of a pharmaceutical composition comprising an absorbable carrier of a biocompatible, biodegradable polymer, and optionally dispersed at least partially through, in or on said absorbable carrier, a sterile powder composition comprising thrombin, wherein said sterile powder composition comprises; (i) a composite particle comprising thrombin and fibrinogen, and/or (ii) a mixture of particles that comprise fibrinogen in the absence of thrombin, and particles that comprises thrombin in the absence of fibrinogen, optionally wherein the sterile powder further comprises a pharmaceutically acceptable excipient or carrier, or amorphous material or polyol, or additive material as described above, wherein said sterile powder is dispersed and/or fixed, at least partially through or on said absorbable carrier, and optionally wherein said sterile powder or pharmaceutical composition comprises fibrinogen in an amount of from about 0.1-15 mg/cm.sup.2 or about 0.5 to 5 mg/cm.sup.2, and/or thrombin in an amount of from about 0.01 to 500 IU/cm.sup.2, or about 0.1 to 50 IU/cm.sup.2, as a hemostat, wherein application of the pharmaceutical composition to a wound results in a post-treatment blood loss of less than about 100 ml/kg, or less than about 80 ml/kg or less than about 60 ml/kg or less than about 40 ml/kg, optionally wherein the pre-treatment blood loss is at least about 5 ml/kg or at least about 10 ml/kg or at least about 15 ml/kg or more.

In a further embodiment of the invention is provided a method of treating a wound or reducing bleeding at a haemorrhaging site, or the use of a pharmaceutical composition comprising an absorbable carrier of a biocompatible, biodegradable polymer, and optionally dispersed at least partially through, in or on said absorbable carrier, a sterile powder composition comprising thrombin, wherein said sterile powder composition comprises; (i) a composite particle comprising thrombin and fibrinogen, and/or (ii) a mixture of particles that comprise fibrinogen in the absence of thrombin, and particles that comprises thrombin in the absence of fibrinogen, optionally wherein the sterile powder further comprises a pharmaceutically acceptable excipient or carrier, or amorphous material or polyol, or additive material as described above, wherein said sterile powder is dispersed and/or fixed, at least partially through or on said absorbable carrier, and wherein said sterile powder or pharmaceutical composition comprises fibrinogen in an amount of from about 0.1-15 mg/cm.sup.2 or about 0.5 to 5 mg/cm.sup.2, and/or thrombin in an amount of from about 0.01 to 500 IU/cm.sup.2, or about 0.1 to 50 IU/cm.sup.2, as a hemostat, wherein application of the pharmaceutical composition to a wound results in a reduction in Mean Arterial Pressure, 60 minutes post injury and relative to pre-injury, of less than about 40 mmHg, or less than about 30 mmHg, or less than about 20 mmHg, or less than about 10 mmHg or less than about 5 mmHg or less than about 3 mmHg, or even no change in MAP relative to pre-injury MAP.

In a further embodiment of the invention is provided a method of treating a wound or reducing bleeding at a haemorrhaging site, or the use of a pharmaceutical composition comprising an absorbable carrier of a biocompatible, biodegradable polymer, and optionally dispersed at least partially through, in or on said absorbable carrier, a sterile powder composition comprising thrombin, wherein said sterile powder composition comprises; (i) a composite particle comprising thrombin and fibrinogen, and/or (ii) a mixture of particles that comprise fibrinogen in the absence of thrombin, and particles that comprises thrombin in the absence of fibrinogen, optionally wherein the sterile powder further comprises a pharmaceutically acceptable excipient or carrier, or amorphous material or polyol, or additive material as described above, wherein said sterile powder is dispersed and/or fixed, at least partially through or on said absorbable carrier, and wherein said sterile powder or pharmaceutical composition optionally comprises fibrinogen in an amount of from about 0.1-15 mg/cm.sup.2 or about 0.5 to 5 mg/cm.sup.2, and/or thrombin in an amount of from about 0.01 to 500 IU/cm.sup.2, or about 0.1 to 50 IU/cm.sup.2, as a hemostat wherein application of the pharmaceutical composition to a wound results in a survival time of at least about 60 minutes, or at least about 90 minutes, or at least about 120 minutes, or at least about 180 minutes, or more.

In a further embodiment of the invention is provided a method of treating a wound or reducing bleeding at a haemorrhaging site, or the use of a pharmaceutical composition comprising an absorbable carrier of a biocompatible, biodegradable polymer, and optionally dispersed at least partially through, in or on said absorbable carrier, a sterile powder composition comprising thrombin, wherein said sterile powder composition comprises; (i) a composite particle comprising thrombin and fibrinogen, and/or (ii) a mixture of particles that comprise fibrinogen in the absence of thrombin, and particles that comprises thrombin in the absence of fibrinogen, optionally wherein the sterile powder further comprises a pharmaceutically acceptable excipient or carrier, or amorphous material or polyol, or additive material as described above, wherein said sterile powder is dispersed and/or fixed, at least partially through or on said absorbable carrier, and wherein said sterile powder or pharmaceutical composition comprises fibrinogen in an amount of from about 0.1-15 mg/cm.sup.2 or about 0.5 to 5 mg/cm.sup.2, and/or thrombin in an amount of from about 0.01 to 500 IU/cm.sup.2, or about 0.1 to 50 IU/cm.sup.2, as a hemostat, wherein application of the pharmaceutical composition to a wound results in a percent survival of at least about 10 percent, or at least about 20 percent, or at least about 30 percent, or at least about 40 percent, or at least about 50 percent, or at least about 75 percent, or at least about 90 percent, or at least about 95 percent, or more.

In a further embodiment of the invention is provided a method of treating a wound or reducing bleeding at a haemorrhaging site, or the use of a pharmaceutical composition comprising an absorbable carrier of a biocompatible, biodegradable polymer, and optionally dispersed at least partially through, in or on said absorbable carrier, a sterile powder composition comprising thrombin, wherein said sterile powder composition comprises; (i) a composite particle comprising thrombin and fibrinogen, and/or (ii) a mixture of particles that comprise fibrinogen in the absence of thrombin, and particles that comprises thrombin in the absence of fibrinogen, optionally wherein the sterile powder further comprise a glassy carrier, wherein said sterile powder is dispersed and/or fixed, at least partially through or on said absorbable carrier, and wherein said sterile powder or pharmaceutical composition comprises fibrinogen in an amount of from about 0.1-15 mg/cm.sup.2 or about 0.5 to 5 mg/cm.sup.2, and/or thrombin in an amount of from about 0.01 to 500 IU/cm.sup.2, or about 0.1 to 50 IU/cm.sup.2, wherein said sterile powder further comprises a pharmaceutically acceptable excipient or carrier, or amorphous material or polyol, or additive material as described above, for hemostasis, tissue sealing and tissue gluing, wherein application or use of the pharmaceutical composition further comprises the application of moderate manual pressure for not less than about 30 seconds, or not less than about 60 seconds, or not less than about 2 minutes, or not less than about 3 minutes, or not less than about 5 minutes, or not less than about 7 minutes, or not less than about 10 minutes, or longer.

In a further embodiment of the invention is provided a method of treating a wound or reducing bleeding at a haemorrhaging site, or the use of a pharmaceutical composition comprising an absorbable carrier of a biocompatible, biodegradable polymer, and optionally dispersed at least partially through, in or on said absorbable carrier, a sterile powder composition comprising thrombin, wherein said sterile powder composition comprises; (i) a composite particle comprising thrombin and fibrinogen, and/or (ii) a mixture of particles that comprise fibrinogen in the absence of thrombin, and particles that comprises thrombin in the absence of fibrinogen, optionally wherein the sterile powder further comprise a pharmaceutically acceptable excipient or carrier, or amorphous material or polyol, or additive material as described above, wherein said sterile powder is dispersed and/or fixed, at least partially through or on said absorbable carrier, and wherein said sterile powder or pharmaceutical composition comprises fibrinogen in an amount of from about 0.1-15 mg/cm.sup.2 or about 0.5 to 5 mg/cm.sup.2, and/or thrombin in an amount of from about 0.01 to 500 IU/cm.sup.2, or about 0.1 to 50 IU/cm.sup.2, for hemostasis, tissue sealing and tissue gluing, wherein application or use of the pharmaceutical composition further comprises the application of moderate manual pressure for not less than about 30 seconds, or not less than about 60 seconds, or not less than about 2 minutes, or not less than about 3 minutes, or not less than about 5 minutes, or not less than about 7 minutes, or not less than about 10 minutes, or longer, and wherein said treatment results in a time to hemostasis (TTH) of less than about 10 minutes, or less than about 8 minutes, or less than about 5 minutes, or less than about 4 minutes, or less than about 3 minutes, or less than about 2 minutes, when administered to a wound which exhibits a bleeding rate of greater than about 30 g/minute, or more.

Another embodiment of the invention is the use of a pharmaceutical composition comprising an absorbable carrier of a biocompatible, biodegradable polymer, and optionally dispersed at least partially through, in or on said absorbable carrier, a sterile powder composition comprising thrombin, wherein said sterile powder composition comprises; (i) a composite particle comprising thrombin and fibrinogen, and/or (ii) a mixture of particles that comprise fibrinogen in the absence of thrombin, and particles that comprises thrombin in the absence of fibrinogen, optionally wherein the sterile powder further comprises a pharmaceutically acceptable excipient or carrier, or amorphous material or polyol, or additive material as described above, wherein said sterile powder is optionally dispersed and/or fixed, at least partially through or on said absorbable carrier, and wherein said sterile powder or pharmaceutical composition comprises fibrinogen in an amount of from about 0.1-15 mg/cm.sup.2 or about 0.5 to 5 mg/cm.sup.2, and/or thrombin in an amount of from about 0.01 to 500 IU/cm.sup.2, or about 0.1 to 50 IU/cm.sup.2, for the treatment of mild to moderate bleeding. Mild bleeds are those typically presenting with a blood flow of less than about 5 g/minute whereas moderate bleeds are often about 10 g/minute or less, or about 20 g/minute, or less, optionally with a TTH of less than 10 minutes, or about 5 minutes or less.

In another embodiment of the invention is provided the use of a pharmaceutical composition comprising an absorbable carrier of a biocompatible, biodegradable polymer, and optionally dispersed at least partially through, in or on said absorbable carrier, a sterile powder composition comprising thrombin, wherein said sterile powder composition comprises; (i) a composite particle comprising thrombin and fibrinogen, and/or (ii) a mixture of particles that comprise fibrinogen in the absence of thrombin, and particles that comprises thrombin in the absence of fibrinogen, optionally wherein the sterile powder further comprises a pharmaceutically acceptable excipient or carrier, or amorphous material or polyol, or additive material as described above, wherein said sterile powder is dispersed and/or fixed, at least partially through or on said absorbable carrier, and wherein said sterile powder or pharmaceutical composition comprises fibrinogen in an amount of from about 0.1-15 mg/cm.sup.2 or about 0.5 to 5 mg/cm.sup.2, and/or thrombin in an amount of from about 0.01 to 500 IU/cm.sup.2, or about 0.1 to 50 IU/cm.sup.2, for the treatment severe bleeding. Severe bleeds are those typically presenting with a blood flow or loss of more than about 30 g/minute, or more than about 40 g/minute, or more than about 50 g/minute, or more than about 60 g/minute, or more than about 100 g/minute, or even more than about 150 g/minute or greater. Accordingly, there is provided composition for treatment of severe or uncontrolled bleeding in a subject in need thereof, wherein the blood flow or loss in said subject is more than about 30 g/minute, or more than about 40 g/minute, or more than about 50 g/minute, or more than about 60 g/minute, or more than about 100 g/minute, or even more than about 150 g/minute or greater, and optionally wherein said treatment results in a TTH of less than about 10 minutes, or less than about 5 minutes or less. In situations where there is such a great rate of bleeding, it is common for there to be a concomitant need for transfusions of blood products and/or infusion of volume expanders, etc.

In another embodiment of the invention, is provided a pharmaceutical composition comprising an absorbable carrier of a biocompatible, biodegradable polymer, and optionally dispersed at least partially through, in or on said absorbable carrier, a sterile powder composition comprising thrombin, wherein said sterile powder composition comprises; (i) a composite particle comprising thrombin and fibrinogen, and/or (ii) a mixture of particles that comprise fibrinogen in the absence of thrombin, and particles that comprises thrombin in the absence of fibrinogen, optionally wherein the sterile powder further comprise a glassy carrier, wherein said sterile powder is dispersed and/or fixed, at least partially through or on said absorbable carrier, and wherein said sterile powder or pharmaceutical composition comprises fibrinogen in an amount of from about 0.1-15 mg/cm.sup.2 or about 0.5 to 5 mg/cm.sup.2, and/or thrombin in an amount of from about 0.01 to 500 IU/cm.sup.2, or about 0.1 to 50 IU/cm.sup.2, wherein said sterile powder further comprises a pharmaceutically acceptable excipient or carrier, or amorphous material or polyol, or additive material as described above, for treatment of severe or uncontrolled bleeding and/or reducing bleeding at a haemorrhaging site in a subject in need thereof, wherein the blood flow or loss in said subject is more than about 30 g/minute, or more than about 40 g/minute, or more than about 50 g/minute, or more than about 60 g/minute, or more than about 100 g/minute, or even more than about 150 g/minute or greater, and optionally wherein said treatment results in a TTH of less than about 10 minutes, or less than about 5 minutes or less, and wherein the duration of severe or uncontrolled bleeding prior to treatment is at least about 2 minutes, or at least about 5 minutes, or at least about 10 minutes, or more.

In another embodiment of the invention, is provided a method of treatment of severe or uncontrolled bleeding and/or reducing bleeding at a haemorrhaging site in a subject in need thereof by administration of a pharmaceutical composition comprising an absorbable carrier of a biocompatible, biodegradable polymer, and optionally dispersed at least partially through, in or on said absorbable carrier, a sterile powder composition comprising thrombin, wherein said sterile powder composition comprises; (i) a composite particle comprising thrombin and fibrinogen, or (ii) a mixture of particles that comprise fibrinogen in the absence of thrombin, and particles that comprises thrombin in the absence of fibrinogen, optionally wherein the sterile powder further comprise a pharmaceutically acceptable excipient or carrier, or amorphous material or polyol, or additive material as described above, wherein said sterile powder is optionally dispersed and/or fixed, at least partially through or on said absorbable carrier, and wherein said sterile powder or pharmaceutical composition comprises fibrinogen in an amount of from about 0.1-15 mg/cm.sup.2 or about 0.5 to 5 mg/cm.sup.2, and/or thrombin in an amount of from about 0.01 to 500 IU/cm.sup.2, or about 0.1 to 50 IU/cm.sup.2, wherein the blood flow or loss in said subject is more than about 30 g/minute, or more than about 40 g/minute, or more than about 50 g/minute, or more than about 60 g/minute, or more than about 100 g/minute, or even more than about 150 g/minute or greater, and optionally wherein said treatment results in a TTH of less than about 10 minutes, or less than about 5 minutes or less, and wherein the duration of severe or uncontrolled bleeding prior to treatment is at least about 2 minutes, or at least about 5 minutes, or at least about 10 minutes, or more.

Another embodiment of the present invention provides a pharmaceutical composition comprising an absorbable carrier of a biocompatible, biodegradable polymer, and optionally dispersed at least partially through, in or on said absorbable carrier, a sterile powder composition comprising thrombin, wherein said sterile powder composition comprises; (i) a composite particle comprising thrombin and fibrinogen, or (ii) a mixture of particles that comprise fibrinogen in the absence of thrombin, and particles that comprises thrombin in the absence of fibrinogen, optionally wherein the sterile powder further comprises a pharmaceutically acceptable excipient or carrier, or amorphous material or polyol, or additive material as described above, wherein said sterile powder is dispersed and/or fixed at least partially through or on said absorbable carrier, and wherein said sterile powder or pharmaceutical composition comprises fibrinogen in an amount of from about 0.1-15 mg/cm.sup.2 or about 0.5 to 5 mg/cm.sup.2, and/or thrombin in an amount of from about 0.01 to 500 IU/cm.sup.2, or about 0.1 to 50 IU/cm.sup.2, wherein said pharmaceutical composition is adapted to form a seal on non-superficial tissues or to close open tissues exceeding minor abrasions, cuts, scrapes, scratches, burns, sunburns, ulcers and other skin injuries and irritations. Treatable wounds include: topical wounds; deeper wounds; surgical incisions; severe wounds; battlefield wounds and trauma; and emergency room excessive bleeding, among others. Accordingly, the various applications of the wound sealants include first aid and triage applications for surgical and medical procedures.

Such pharmaceutical compositions may be administered during or after surgery, or may be administered to the wound or wounds of a subject, including human, mammal and other veterinary applications, as described above.

For the avoidance of doubt, the amount of thrombin referred to in embodiments of the invention, may be defined as an amount as measured by activity or potency, as defined herein.

The invention further comprises a method of treating a wound or for reducing bleeding at a haemorrhaging site by applying or administering any pharmaceutical composition as described above.

For the avoidance of doubt the invention relates to compositions as described herein for use in medicine, use of said compositions in the preparation of medicaments for the medical uses as described herein and methods of medical treatment comprising use of compositions as described herein.

In a further embodiment of the invention, the above pharmaceutical composition may be formulated and packaged as a sterile preparation for single-delivery application to a wound site, or as a multi-use preparation.

In a further embodiment of the invention, the invention relates to sterile, packaged thrombin and fibrinogen particles, optionally in combination, suitably produced by spray drying and suitably not irradiated.

In a further embodiment of the invention, the above pharmaceutical composition may be applied topically to a wound site. Alternatively or in addition, the composition can be introduced internally into the wound site in the case of, for example, deeper lacerations, arterial wounds, or during surgical procedures.

In another embodiment of the invention is provided a pharmaceutical composition comprising an absorbable carrier of a biocompatible, biodegradable polymer, and optionally dispersed at least partially through, in or on said absorbable carrier, a sterile powder composition comprising thrombin, wherein said sterile powder composition comprises; (i) a composite particle comprising thrombin and fibrinogen, and/or (ii) a mixture of particles that comprise fibrinogen in the absence of thrombin, and particles that comprises thrombin in the absence of fibrinogen, optionally wherein the sterile powder further comprises a pharmaceutically acceptable excipient or carrier, or amorphous material or polyol, or additive material as described above, wherein said sterile powder is dispersed and/or fixed, at least partially through or on said absorbable carrier, and wherein said sterile powder or pharmaceutical composition comprises fibrinogen in an amount of from about 0.1-15 mg/cm.sup.2 or about 0.5 to 5 mg/cm.sup.2, and/or thrombin in an amount of from about 0.01 to 500 IU/cm.sup.2, or about 0.1 to 50 IU/cm.sup.2, for use in surgical interventions such as such as in the gastrointestinal system, e.g. the oesophagus, stomach, small intestine, large intestine, bowel, rectum, on parenchymal organs such as the liver, pancreas, spleen, lungs, kidney, adrenal glands, lymph and thyroid glands; surgical interventions in the ear, nose and throat area (ENT) including dental surgery, epistaxis, cardiovascular surgery, such as carotid endarterectomy, femoropopliteal bypass or coronary artery bypass grafting (CABG); aesthetic surgery, spinal surgery, neurological surgery, such as posterior lumbar interbody fusion, microdiscectomy or craniotomy; lymphatic, biliary, and cerebrospinal (CSF) fistulae, air leakages during thoracic and pulmonary surgery, thoracic surgery including surgery of the trachea, bronchi and lungs; orthopaedic surgery, such as knee or hip replacement; gynaecological surgical procedures such as caesarian section, hysterectomy, fibroid surgery; vascular surgery, such as shunts; urological, bone (e.g. spongiosa resection), and emergency surgery. Suitable surgical interventions include orthopaedic surgery, liver resection, soft tissue injury/surgery and vascular surgery. The pharmaceutical composition may be applied with a layer of the sterile powder composition, if present, adjacent to the wound surface, or wherein the layer is on the opposite side to that applied to the wound surface.

In another embodiment of the invention is provided a pharmaceutical composition comprising an absorbable carrier of a biocompatible, biodegradable polymer, and optionally dispersed at least partially through, in or on said absorbable carrier, a sterile powder composition comprising thrombin, wherein said sterile powder composition comprises; (i) a composite particle comprising thrombin and fibrinogen, and/or (ii) a mixture of particles that comprise fibrinogen in the absence of thrombin, and particles that comprises thrombin in the absence of fibrinogen, optionally wherein the sterile powder further comprises a pharmaceutically acceptable excipient or carrier, or amorphous material or polyol, or additive material as described above, wherein said sterile powder is dispersed and/or fixed, at least partially through or on said absorbable carrier, and wherein said sterile powder or pharmaceutical composition comprises fibrinogen in an amount of from about 0.1-15 mg/cm.sup.2 or about 0.5 to 5 mg/cm.sup.2, and/or thrombin in an amount of from about 0.01 to 500 IU/cm.sup.2, or about 0.1 to 50 IU/cm.sup.2, wherein the absorbable carrier comprises a biocompatible, biodegradable polymer selected from the group consisting of polysaccharides, albumin, a cellulose, methylcellulose, alkylhydroxyalkyl cellulose, hydroxyalkyl cellulose, cellulose sulfate, salts of carboxymethyl cellulose, carboxymethyl cellulose, carboxyethyl cellulose, oxidised cellulose; gelatins or collagen, such as a collagen-sponge, chitin, carboxymethyl chitin, hyaluronic acid, salts of hyaluronic acid, alginate, alginic acid, propylene glycol alginate, glycogen, dextran, dextran sulfate, curdlan, pectin, pullulan, xanthan, chondroitin, chondroitin sulfates, carboxymethyl dextran, carboxymethyl chitosan, chitosan, heparin, heparin sulfate, heparan, heparan sulfate, dermatan sulfate, keratan sulfate, carrageenans, starch, amylose, amylopectin, poly-N-glucosamine, poly-N-acetyl glucosamine, polymannuronic acid, polyglucuronic acid, polyguluronic acid, chitosan, carboxymethyl chitosan, chitosan salts; a polyurethane; oxidised polysaccharides, and derivatives or combinations of any of the above.

Other suitable biocompatible, biodegradable polymers for use in this embodiment of the invention, include polyurethanes and absorbable carriers formed from a polyurethane, such as those disclosed in WO 2004/062704 and WO 2010/137981 (Polyganics B.V), herein incorporated by reference.

Other biocompatible, biodegradable polymers for use in this embodiment of the invention, include chitin, chitin-glucan, chitosan, chitosan-glucan, derivatives thereof, and any combinations thereof, and absorbable carriers formed from such polymers and combinations, such as those disclosed in WO 2010/142507 and WO 2007/122187 (Kitozyme S.A), herein incorporated by reference.

In another embodiment of the invention is provided a pharmaceutical composition comprising an absorbable carrier of a biocompatible, biodegradable polymer, and optionally dispersed at least partially through, in or on said absorbable carrier, a sterile powder composition comprising thrombin, wherein said sterile powder composition comprises; (i) a composite particle comprising thrombin and fibrinogen, and/or (ii) a mixture of particles that comprise fibrinogen in the absence of thrombin, and particles that comprises thrombin in the absence of fibrinogen, optionally wherein the sterile powder further comprises a pharmaceutically acceptable excipient or carrier, or amorphous material or polyol, or additive material as described above, wherein said sterile powder is dispersed and/or fixed, at least partially through or on said absorbable carrier, and wherein said sterile powder or pharmaceutical composition comprises fibrinogen in an amount of from about 0.1-15 mg/cm.sup.2 or about 0.5 to 5 mg/cm.sup.2, and/or thrombin in an amount of from about 0.01 to 500 IU/cm.sup.2, or about 0.1 to 50 IU/cm.sup.2, wherein the absorbable carrier comprises a support material, such as a gauze, sponge, pad, bandage and the like.

In one embodiment a choice of application or incorporation of said sterile powder and carrier may be selected from spraying, dripping, dipping, impregnation or otherwise embedding or applying said sterile powder or composition, and/or blend thereof, in predetermined strengths such as for example: 20%, 40%, 60% or 80%, or any other suitable strengths, on the support and identify the strength of the sterile powder applied thereon. Methods of application of the powder microparticle components and/or blend thereof, or combinations of the microparticle components and other materials as described herein, into, onto or throughout the support material, are well known to those skilled in the art.

Suitable vehicles for use in the method of this embodiment include, but are not limited to carriers, solvent, perfluorocarbons and the like. The vehicle may be a solvent or mixture of solvents classified under the ICH Guidelines as either class 2 or Class 3. Suitable such solvents in Class 2 include acetonitrile, cyclohexane, dichloromethane, 1,4-dioxane, ethylene glycol, hexane, methanol, toluene, xylene, and the like. Suitable such solvents in Class 3 include acetone, anisole, 1-butanol, 2-butanol, butyl acetate, heptane, isopropyl acetate, methylethyl ketone, 2-methyl-1-propanol, dimethylsulfoxide, ethanol, ethyl acetate, ethyl ether, ethyl formate, pentane, 1-pentanol, 2-propanol, and the like. In one method is the use of ethanol and/or 1,4-dioxane.

In another embodiment of the invention is provided a method of making a pharmaceutical composition comprising an absorbable carrier of a biocompatible, biodegradable polymer, and optionally dispersed at least partially through, in or on said absorbable carrier, a sterile powder composition comprising thrombin, wherein said sterile powder composition comprises; (i) a composite particle comprising thrombin and fibrinogen, and/or (ii) a mixture of particles that comprise fibrinogen in the absence of thrombin, and particles that comprises thrombin in the absence of fibrinogen, optionally wherein the sterile powder further comprise a pharmaceutically acceptable excipient or carrier, or amorphous material or polyol, or additive material as described above, wherein said sterile powder is dispersed and/or fixed, at least partially through or on said absorbable carrier, and wherein said sterile powder or pharmaceutical composition comprises fibrinogen in an amount of from about 0.1-15 mg/cm.sup.2 or about 0.5 to 5 mg/cm.sup.2, and/or thrombin in an amount of from about 0.01 to 500 IU/cm.sup.2, or about 0.1 to 50 IU/cm.sup.2, said method includes the steps of; (i), forming a dispersion or suspension of said mixture of microparticles in a vehicle or carrier fluid in which they do not dissolve, optionally comprising a binding or viscosifying agent, (ii) applying said dispersion or suspension to one or more surfaces of the absorbable carrier, under atmospheric, reduced or elevated pressure, and optionally (iii), removing said vehicle.

Suitable processes for applying said dispersion or suspension to impregnate said carrier include percolation, spraying, dipping, soaking, dripping, impregnating, embedding, vacuum pressure impregnation, high pressure impregnation, and the like. Alternatively, a sandwich presentation may be formed by the application of the dispersion or suspension comprising a binding agent to the surface or surfaces of one or more separate carrier/matrices and adjoining them together before the optional removal of the vehicle or carrier fluid.

Suitable methods of removing said vehicle in step (iii) above are well known to those skilled in the art but include, but are not limited to, air drying, freeze-drying, vacuum drying (optionally at elevated humidity), microwave vacuum drying, supercritical processing (such as RES, SEDS, etc), forced air drying, and the like.

Suitable binding and/or viscosifying agents are known in the art, which may include amphiphilic polymers such as hydropxypropyl cellulose, or PVP and the like. In this way, the suspension may demonstrate enhanced or adequate dispersion stability and thereby may ensure a consistent dosing and homogeneity of application under step (ii) above, and may maintain content uniformity within the composition.

In another embodiment of the invention is provided a method of making a pharmaceutical composition according to the invention which comprises an absorbable carrier of a biocompatible, biodegradable polymer, and optionally dispersed at least partially through, in or on said absorbable carrier, a sterile powder composition comprising thrombin, wherein said sterile powder composition comprises; (i) a composite particle comprising thrombin and fibrinogen, or (ii) a mixture of particles that comprise fibrinogen in the absence of thrombin, and particles that comprises thrombin in the absence of fibrinogen, optionally wherein the sterile powder further comprises a pharmaceutically acceptable excipient or carrier, or amorphous material or polyol, or additive material as described above, wherein said sterile powder is dispersed and/or fixed, at least partially through or on said absorbable carrier, and wherein said sterile powder or pharmaceutical composition comprises fibrinogen in an amount of from about 0.1-15 mg/cm.sup.2 or about 0.5 to 5 mg/cm.sup.2, and/or thrombin in an amount of from about 0.01 to 500 IU/cm.sup.2, or about 0.1 to 50 IU/cm.sup.2, wherein the absorbable carrier comprises a support material, such as a gauze, sponge, pad, bandage and the like, and the method includes the steps of; (i), forming a dispersion or optionally homogenous suspension of said mixture of microparticles or blend thereof, in a vehicle or carrier fluid in which they do not dissolve, together with an appropriate amount of a biocompatible, biodegradable polymer in solution or suspension, optionally further comprising a binding or viscosifying agent, and (ii), removing said vehicle. In this way, a composite of the absorbable carrier comprising said sterile powder composition is formed with said microparticles entrapped with, on or throughout said absorbable carrier. The method of removal of said vehicle or carrier liquid greatly influences the nature of the final composition obtained. For example, freeze-drying can result in a porous matrix of the biocompatible, biodegradable polymer and/or composition according to the invention, whereas simple air-drying can result in a film of said biocompatible, biodegradable polymer and/or composition according to the invention. Suitable concentration ranges for the solution of the biocompatible, biodegradable polymer include about 0.1 to 70% w/v, or about 0.5 to 50% w/v, or about 0.75 to 10% w/v, or about 1 to 5% w/v. Suitable concentration ranges for the solution of the binder or viscosifying agent include about 0.001 to 50% w/v, or about 0.01 to 10% w/v, or about 0.1 to 5% w/v, or about 1 to 3% w/v.

Suitable methods of removing said vehicle in step (ii) are well known to those skilled in the art but include, but are not limited to, include air drying, freeze-drying, vacuum drying optionally at elevated humidity, microwave vacuum drying, supercritical processing (such as RES, SEDS, etc.), forced air drying, and the like. Optionally, the residual amount of vehicle, moisture content, carrier fluid, solvent or the like, may be reduced to an acceptable or appropriate level.

The method of manufacture described above may be performed under sterile or aseptic conditions, so as to avoid the need for terminal sterilisation of the composition using gamma irradiation, electron-beam sterilisation, or treatment with ethylene oxide, or other such techniques known to those skilled in the art.

As used in this embodiment, “moisture content” or “residual solvent” refers to the amount freely-available water or solvent or the like. “Freely-available” means the residue is not bound to or complexed with one or more of the non-liquid components of a composition according to the invention. The moisture content referenced herein refers to levels determined by procedures such as modified Karl Fischer method or by near infrared spectroscopy. Suitable moisture content(s) or residual solvent levels for a particular composition according to the invention may be determined empirically by one skilled in the art depending upon the intended application(s) thereof. For example, in this embodiment, higher moisture or solvent contents may be associated with more flexible compositions according to the invention. Thus, in certain applications is moisture content of at least about 3% to about 6% by weight or even in the range of about 6% to 44% by weight. In other embodiments, lower moisture contents are associated with more rigid compositions. Thus, in applications for wounds to the abdomen or chest, for example, the moisture content may be less than about 6% by weight or even in the range of about 1% to about 6% by weight of a composition according to this embodiment.

Thus, suitable ranges of moisture or residual solvent contents for compositions according to this embodiment of the invention include, but are not limited to, the following (each value being .+−0.0.9%): less than 53%; less than 44%; less than 28%; less than 24%; less than 16%; less than 12%; less than 6%; less than 5%; less than 4%; less than 3%; less than 2.5%; less than 2%; less than 1.4%; between 0 and 12%, non-inclusive; between 0 and 6%; between 0 and 4%; between 0 and 3%; between 0 and 2%; between 0 and 1%; between 1 and 16%; between 1 and 11%; between 1 and 8%; between 1 and 6%; between 1 and 4%; between 1 and 3%; between 1 and 2%; and between 2 and 4%, by weight of the composition.

In a further embodiment of the invention, the sterile powder composition comprising thrombin as incorporated into said pharmaceutical compositions may be dispersed at least partially through, in or on said absorbable carrier at a concentration per unit area of the carrier ranging from about 1 mg of said mixture or optionally homogenous blend thereof, per square cm, to about 5000 mg per square cm, or about from 10 mg per square cm to about 2000 mg per square cm, more suitably from about 25 mg per square cm to about 500 mg per square cm. Optionally, said sterile powder composition may optionally be homogenously distributed and/or fixed through, in or on said absorbable carrier, such as fixed on. With reference to the term “fixed” it is intended to mean that the microparticles are attached, glued, fused, embedded, dried in, on or through, or bound or in any other way and/or connected to the carrier such that they do not readily detach during transit and/or use. A suitable test for such fixation is one which determines the amount of microparticles removed after exposure of the composition of the invention to shaking or vibration. For examples, a relevant procedure is one such as that found in U.S. Pat. No. 7,052,713, wherein the assessment of the strength of fixation of microparticles to a carrier is such that abrasion of said composition is less than 1.0 mg/cm.sup.2 when a sample of said composition is shaken on a Vibrofix shaker at a frequency of about 1000 rpm for 2 minutes Accordingly, compositions according to the invention, when subjected to the same test procedure, exhibit an abrasion of less than about 50 mg/cm.sup.2, or less than about 40 mg/cm.sup.2, or less than about 30 mg/cm.sup.2, or less than about 20 mg/cm.sup.2, or less than about 10 mg/cm.sup.2, or even less than about 2 mg/cm.sup.2.

In another embodiment of the invention is provided a method of making a pharmaceutical composition comprising an absorbable carrier of a biocompatible, biodegradable polymer, and optionally dispersed at least partially through, in or on said absorbable carrier, a sterile powder composition comprising thrombin, wherein said sterile powder composition comprises; (i) a composite particle comprising thrombin and fibrinogen, or (ii) a mixture of particles that comprise fibrinogen in the absence of thrombin, and particles that comprises thrombin in the absence of fibrinogen, optionally wherein the sterile powder further comprise a pharmaceutically acceptable excipient or carrier, or amorphous material or polyol, or additive material as described above, wherein said sterile powder is dispersed and/or fixed, at least partially through or on said absorbable carrier, and wherein said sterile powder or pharmaceutical composition comprises fibrinogen in an amount of from about 0.1-15 mg/cm.sup.2 or about 0.5 to 5 mg/cm.sup.2, and/or thrombin in an amount of from about 0.01 to 500 IU/cm.sup.2, or about 0.1 to 50 IU/cm.sup.2, optionally wherein said pharmaceutical composition has a porosity or void fraction of between 1 and 99.9%, or about between 5 and 99%, or about between 10 and 98%, or about between 15 and 95%, wherein the porosity or void fraction is the fraction of the volume of voids over the total volume, expressed as a percentage. Alternatively, pores when present in the composition may have a diameter of from about 0.5 microns to about 5 mm, or from about 1 micron to about 1 mm or even from about 10 microns to about 500 microns.

In another embodiment of the invention is provided a method of making a pharmaceutical composition comprising an absorbable carrier of a biocompatible, biodegradable polymer, and dispersed at least partially through, in or on said absorbable carrier, a sterile powder composition comprising thrombin, wherein said sterile powder composition comprises; (i) a composite particle comprising thrombin and fibrinogen, or (ii) a mixture of particles that comprise fibrinogen in the absence of thrombin, and particles that comprises thrombin in the absence of fibrinogen, optionally wherein either or both first and/or second microparticles further comprise a pharmaceutically acceptable excipient or carrier, or amorphous material or polyol, or additive material as described above, wherein the pharmaceutical composition demonstrates an absorption capacity to take up more than about 10, or about 20, or about 30, or about 40 or about 50 or even about 100 times or more, its own weight in blood or other body fluids. Such impregnated supports and sponges and the like allow the blood to seep into the structure before or whilst clotting occurs.

The marking of the pharmaceutical composition according to this embodiment may take the form of imprinting the percentage strength, e.g., 20%, 40% or 60%, on the surface (on one or both sides) or just underneath one layer thereof. The percentage markings may be any other suitable FIGURES, such as 25%, 50% and 75%, or even 1, 2, 3, 4, 5, as desired. The dimensions of the pharmaceutical composition may be any such size, area and volume as required for a particular application or bleeding rate and would be apparent to those skilled in the art. These may include 1.times.1 cm, 2.times.2 cm, 3.times.3 cm, and so forth.

The thickness of the pharmaceutical composition may be adapted for a particular application or bleeding rate and would be apparent to those skilled in the art, but may include between about 0.1 cm and 10 cm thick, or about 0.5 to 5 cm, for single or sandwich compositions.

In one aspect the pharmaceutical compositions of the invention exhibit synergy and/or greater efficacy compared to the sterile powder alone or a blend thereof, by encouraging clot formation at the interface of the wound and the composition where the dry powder or microparticle components and/or blend thereof has been embedded and/or coated.

In another embodiment of the invention comprises a support material, such as a gauze, sponge, bandage and the like, it is proposed herein to spray or otherwise embed or apply the composition of the invention such as a hemostat or fibrin sealant powder composition in predetermined strengths such as for example: 20%, 40%, 60% or 80%, or any other preferred strengths, on the support and identify the strength of the composition of the invention such as a hemostat or fibrin sealant powder applied thereon. Methods of application of said compositions of the invention such as a pharmaceutical composition, or combinations of the fibrin sealant powder composition and other materials, into the treated support, are well known to those skilled in the art. The marking of the treated support could take the form of imprinting the percentage strength, e.g., 20%, 40% or 60%, on the surface of the treated support (on one or both sides) or just underneath one layer thereof. The percentage markings may be any other preferred FIGURES, such as 25%, 50% and 75%, or even 1, 2, 3, 4, 5, as desired. After the marking is done, the treated support is subjected to sterilization as desired.

Such novel presentations of the sterile powder composition or pharmaceutical composition comprising thrombin according to the invention, optionally further comprising fibrinogen, may exhibit synergy and/or greater efficacy compared to the powder alone, by encouraging clot formation at the interface of the wound and device where the dry powder fibrin sealant has been embedded and/or coated. Existing impregnated supports and sponges and the like allow the blood to seep into the structure before clotting occurs.

Other suitable methods or uses of the invention are for sealing incisions, perforations, and/or fluid or gaseous leaks in biological tissues during a surgical procedure, and comprise contacting the tissue with a composition according to the invention, thereby sealing the incision, perforation, or fluid or gaseous leak.

In an embodiment the invention relates to a pharmaceutical composition or sterile powder composition suitable for medical use comprising thrombin, optionally further comprising fibrinogen, either or both of which may optionally be water-soluble, and/or wherein said sterile powder composition and/or fibrinogen may be obtained by spray-drying a suitable solution or suspension, wherein the product obtained may be free-flowing, discrete and optionally substantially dry or anhydrous, with a residual water or moisture content optionally no greater than about 8% w/w or about 5% w/w, or about 3% w/w. As such, the pharmaceutical composition or a sterile powder composition comprising thrombin in accordance with this invention, optionally further comprising fibrinogen, and/or components thereof, may not activated until they are wetted, e.g. by coming into contact with liquid at a wound site. The pharmaceutical composition or sterile powder composition suitable for medical use comprising thrombin, optionally further comprising fibrinogen, may be delivered as a dry mixture, although separate application of the thrombin-containing composition or fibrinogen, when present, is also envisaged.

In another embodiment the invention relates to a sterile powder composition comprising fibrinogen, thrombin, albumin, sodium chloride, sodium citrate and arginine HCl.

In another embodiment, the invention relates to a sterile powder composition as set out below, per 1 gram of powder:

| | | |
|---|---|---|
| Human Fibrinogen | 71 | mg/g |
| Human Thrombin | 643 | IU/g |
| Trehalose | 0.815 | g/g^{a} |
| Calcium chloride | 11.25 | mg/g |
| Albumin | 41.3 | mg/g^{b} |
| Sodium chloride | 20.9 | mg/g^{b} |
| Sodium citrate | 5.8 | mg/g^{b} |
| L-Arginine hydrochloride | 38.8 | mg/g^{b} |

^{a}Nominal value based on target specifications of actives, actual value is calculated per lot active
^{b}100% theoretical calculation (not determined) using the target specification as supplied on the Certificate of Analysis of the drug substances

In a further embodiment the invention relates to a sterile powder composition suitable for medical use comprising thrombin and fibrinogen, wherein the powder exhibits a thrombin potency or activity of at least 500 IU's per gram sterile powder composition, and/or a fibrinogen content of at least 50 mg per gram sterile powder composition, and/or wherein the powder comprises 60 to 130 mg protein per gram sterile powder composition, and/or wherein the powder comprises not more than 6% w/w residual water, and/or wherein said powder exhibits a crystalline purity of not less more than 15% w/w, and/or a median diameter between 10 and 100 microns, optionally when assessed as an average of at least 3 different batches, optionally up to 10 different batches, and wherein said pharmaceutical composition or powder composition additionally comprises an amorphous material, which material such as a polymer, amino acid, protein or saccharide, such as trehalose, or combination thereof, optionally wherein the material or combination of materials is substantially in glass form at 20 degrees C.

In one embodiment the invention relates to a sterile powder composition suitable for medical use comprising thrombin and fibrinogen, wherein the powder exhibits a thrombin potency or activity of at least 500 IU's per gram sterile powder composition, and a fibrinogen content of at least 50 mg per gram sterile powder composition, and wherein the powder comprises 60 to 130 mg protein per gram sterile powder composition, and wherein the powder comprises not more than 6% w/w residual water and wherein said powder exhibits a crystalline purity of not less more than 15% w/w, and a median diameter between 10 and 100 microns, optionally when assessed as an average of at least 3 different batches, optionally up to 10 different batches, and wherein said pharmaceutical composition or powder composition additionally comprises an amorphous material, which material is a polymer, amino acid, protein or saccharide, such as trehalose, or combination thereof, optionally wherein the material or combination of materials is substantially in glass form at 20 degrees C.

In one embodiment the invention relates to a sterile powder composition suitable for medical use comprising thrombin and fibrinogen, wherein the powder exhibits a thrombin potency or activity of at least 500 IU's per gram sterile powder composition, and a fibrinogen content of at least 50 mg per gram sterile powder composition, and wherein the powder comprises 60 to 130 mg protein per gram sterile powder composition, and wherein the powder comprises not more than 6% w/w residual water and wherein said powder exhibits a crystalline purity of not less more than 15% w/w, and a median diameter between 10 and 100 microns, optionally when assessed as an average of at least 3 different batches, optionally up to 10 different batches, and wherein said pharmaceutical composition or powder composition additionally comprises trehalose.

In a further embodiment of the invention, there is provided a pharmaceutical composition or a sterile powder composition suitable for medical use comprising thrombin, wherein the powder is produced from a feedstock, such as a liquid feedstock, and wherein the powder resulting from removal of liquid from the feedstock exhibits at least 80% of the thrombin activity or potency of the feedstock, and/or wherein the powder exhibits at least 500 IU's of thrombin potency or activity per gram sterile powder composition, optionally when assessed as an average of at least 3 different batches, optionally up to 10 different batches, and wherein said pharmaceutical composition or powder composition additionally comprises an amorphous material, such as a polymer, amino acid, protein or saccharide, such as trehalose, or combination thereof, optionally wherein the material or combination of materials is substantially in glass form at 20 degrees C., wherein the thrombin is provided in combination with fibrinogen, and/or wherein the powder comprises 60 to 130 mg protein/gram composition, and/or a fibrinogen content of at least 50 mg per gram sterile powder composition, and/or wherein the powder comprises not more than 6% w/w residual water, and/or wherein said powder exhibits a crystalline purity of not more than 15% w/w, and/or a median diameter between 10 and 100 microns.

In a further embodiment of the invention, there is provided a pharmaceutical composition or a sterile powder composition suitable for medical use comprising thrombin, wherein the powder is produced from a feedstock, such as a liquid feedstock, and wherein the powder resulting from removal of liquid from the feedstock exhibits at least 80% of the thrombin activity or potency of the feedstock, and wherein the powder exhibits at least 500 IU's of thrombin potency or activity per gram sterile powder composition, optionally when assessed as an average of at least 3 different batches, optionally up to 10 different batches, and wherein said pharmaceutical composition or powder composition additionally comprises an amorphous material, such as a polymer, amino acid, protein or saccharide, such as trehalose, or combination thereof, optionally wherein the material or combination of materials is substantially in glass form at 20 degrees C., wherein the thrombin is provided in combination with fibrinogen, and/or wherein the powder comprises 60 to 130 mg protein/g composition, and a fibrinogen content of at least 50 mg per gram sterile powder composition, and wherein the powder comprises not more than 6% w/w residual water, and wherein said powder exhibits a crystalline purity of not more than 15% w/w, and a median diameter between 10 and 100 microns.

In a further embodiment of the invention, there is provided a method for preparing a sterile powder thrombin composition, the method comprising removing water from an aqueous solution comprising thrombin, wherein removing water is carried out under aseptic conditions, optionally wherein the removal of water is by spray drying or fluid bed drying, and wherein the thrombin is provided in combination with fibrinogen, wherein fibrinogen is spray dried under aseptic conditions to form a powder, thrombin is separately spray dried under aseptic conditions to form a powder and the powders are then combined under aseptic conditions, wherein thrombin is comprised within any or all of the discrete units constituting said powder wherein fibrinogen is comprised within any or all of the discrete units constituting said powder, and the two powders are combined in a weight ratio of about 1:1, and wherein said pharmaceutical composition or powder composition additionally comprises an amorphous material, which may be a polymer, amino acid, protein or saccharide, such as trehalose, or combination thereof, optionally wherein the material or combination of materials is substantially in glass form at 20 degrees C., and wherein the powder exhibits at least 500 IU's of thrombin activity or potency per gram sterile powder composition, and/or wherein the powder comprises 60 to 130 mg total protein/g composition, and/or a fibrinogen content of at least 50 mg per gram sterile powder composition, and/or wherein the powder comprises not more than 6% w/w residual water, and/or wherein said powder exhibits a crystalline purity of not more than 15% w/w, and/or a median diameter between 10 and 100 microns, optionally when assessed as an average of at least 3 different batches, optionally up to 10 different batches.

The invention also includes the following aspects/embodiments/features:

1 A sterile powder composition suitable for medical use comprising thrombin wherein the powder is produced from a feedstock, such as a liquid feedstock, and wherein the powder resulting from removal of liquid from the feedstock exhibits at least 80% of the thrombin potency or activity of the feedstock.

2 A sterile powder composition suitable for medical use comprising thrombin wherein the powder exhibits at least 500 IU's of thrombin potency or activity per gram sterile powder composition, such as at least 600 IUs, 700 IUs, 800, IUs, 900 IUs, 1000 IUs, 1100 IUs, 1200 IUs, 1300 IUs, 1400 IUs, or more, optionally when assessed as an average of at least 3 different batches, optionally up to 10 different batches, optionally wherein the feedstock is made by dissolving or suspending in a liquid a solid having an activity or potency of 1500 IU/gram solid to produce the feedstock.

3 A sterile powder composition according to aspect/embodiment/feature 1 or 2 wherein the powder is produced by removal of liquid by a process selected from spray drying or fluid bed drying.

4 A sterile powder composition according to aspect/embodiment/feature 3 wherein the spray drying is an aseptic spray drying process

5 A sterile powder composition according to any of aspects/embodiments/features 1 to 4 comprising an additional pharmaceutically active agent.

6 A sterile powder composition according to aspect/embodiment/feature 5 wherein the composition comprises fibrinogen powder.

7 A sterile powder composition according to aspect/embodiment/feature 6 wherein the fibrinogen powder is produced by removal of liquid from a feedstock, optionally by spray drying or fluid bed drying.

8 A sterile powder composition according to aspect/embodiment/feature 7 comprising

(i) a composite particle comprising thrombin and fibrinogen, or

(ii) a mixture of

particles that comprise fibrinogen in the absence of thrombin, and

particles that comprises thrombin in the absence of fibrinogen optionally wherein the fibrinogen is co-spray dried with the thrombin to form the composite or mixture.

9 A sterile powder composition according to aspect/embodiment/feature 2 which exhibits at least 1300 IU's of thrombin potency or activity per gram sterile powder composition, such as at least 1400 IUs, or more, optionally when assessed as an average of at least 3 different batches, optionally up to 10 different batches, optionally wherein the feedstock is made by dissolving or suspending in a liquid a solid having an activity or potency of 1500 IU/gram solid to produce the feedstock.

10 A composition of any of aspects/embodiments/features 2 to 9 wherein thrombin activity is measured by a chromogenic assay or wherein potency is measured by a time to clot assay.

11 A composition of any of aspects/embodiments/features 6 to 9 wherein the thrombin is recombinant and/or the fibrinogen is recombinant fibrinogen or variant thereof, such as a fibrinogen in which more than 10% of the alpha, beta or gamma chains are of a variant type, wherein the variant type is preferably a gamma prime chain or an alpha extended chain.

12 A composition of any of aspects/embodiments/features 1 and 3 to 11 wherein the powder resulting from the removal of the liquid from the feedstock exhibits at least 85%, or 90%, or 96% of the thrombin activity or potency of the feedstock.

13 A composition according to any preceding aspect/embodiment/feature, packaged as a sterile final pharmaceutical product for medical use.

14 A composition according to any preceding aspect/embodiment/feature wherein the thrombin is comprised within the powder in the form of nanoparticles, nanofibres, fibres, particles, granules, beads, microbeads, microspheres, microcapsules or microparticles, preferably microparticles.

15 A composition according to any of aspects/embodiments/features 6 to 14 wherein the fibrinogen is comprised within the powder in the form of nanoparticles, nanofibres, fibres, particles, granules, beads, microbeads, microspheres, microcapsules or microparticles, preferably microparticles.

16 A composition according to any preceding aspect/embodiment/feature wherein the feedstock comprises a solution or a suspension of thrombin.

17 A composition according to any preceding aspect/embodiment/feature wherein the liquid present in the feedstock is selected from an aqueous or organic solvent, or mixtures thereof.

18 A pharmaceutical composition comprising a powder composition according to any preceding aspect/embodiment/feature in combination with a pharmaceutically acceptable excipient or carrier, optionally wherein the excipient is in a powder form.

19 A pharmaceutical composition according to aspect/embodiment/feature 18 wherein the excipient or carrier is a biocompatible, biodegradable polymer.

20 A pharmaceutical composition according to aspect/embodiment/feature 18 or 19 wherein the excipient is present in the feedstock or is added to the powder resulting from the feedstock.

21 A pharmaceutical composition or powder composition according to any preceding aspect/embodiment/feature wherein the degree of crystallinity as measured by XRPD or FTIR is not greater than 15% w/w, such as not greater than 10%, 8% or 7%.

22 A pharmaceutical composition or powder composition according to any preceding aspect/embodiment/feature wherein the powder comprises no more than 20% residual water or moisture, optionally no more than 10% residual water or moisture by weight, optionally no more than 6% w/w residual water or moisture.

23 A pharmaceutical composition or powder composition according to any preceding aspect/embodiment/feature wherein the powder comprises 60 to 130 mg protein/gram composition.

24 A pharmaceutical composition or powder composition according to any preceding aspect/embodiment/feature additionally comprising one or more amorphous materials, which may be a polymer, amino acid, protein or saccharide, such as trehalose, including combinations thereof, optionally wherein the material or combination of materials is substantially in glass form at 20 degrees C.

25 A pharmaceutical composition or powder composition according to any preceding aspect/embodiment/feature wherein the composition or powder comprises trehalose, optionally present in an excess of the amount of thrombin by weight, such as in an amount of 10 to 15 times or more than that of the thrombin by weight.

26 A delivery device or container comprising a powder composition or pharmaceutical composition according to any preceding aspect/embodiment/feature, optionally wherein the composition or pharmaceutical composition is approved for clinical use in humans.

27 A method for preparing a sterile powder thrombin composition, the method comprising removing liquid from a solution or suspension comprising thrombin, wherein removal of said liquid is carried out under aseptic conditions.

28 A method according to aspect/embodiment/feature 27 wherein the removal of liquid is by spray drying or fluid bed drying.

29 A method according to aspect/embodiment/feature 27 or 28 wherein the powder is produced from a feedstock and wherein the sterile powder resulting from the feedstock exhibits at least 80% of the thrombin activity or potency of the feedstock.

30 A method according to any of aspects/embodiments/features 27 to 29 wherein the thrombin is provided in combination with fibrinogen.

31 A method according to any of aspects/embodiments/features 27 to 30 wherein the fibrinogen is co-spray dried with the thrombin powder to form either,

(i) a composite particle comprising thrombin and fibrinogen, or

(ii) a mixture of particles that comprise fibrinogen in the absence of thrombin, and particles that comprises thrombin in the absence of fibrinogen.

32 A method according to any of aspects/embodiments/features 27 to 30 wherein fibrinogen is spray dried under aseptic conditions to form a powder, thrombin is separately spray dried under aseptic conditions to form a sterile powder and the powders are combined.

33 The method of aspects/embodiments/features 31 or 32, wherein thrombin is comprised within a microparticle, wherein fibrinogen is comprised within a microparticle and the two types of microparticle are combined in a weight ratio of about 1:1.

34 The method of aspect/embodiment/feature 28, wherein spray drying is undertaken employing one or both of:

an atomisation pressure of at least 0.7 bar;

a feed rate greater than 1300 g/hour

35 A method according to any of aspects/embodiments/features 27 to 34 comprising a further step of packing the powder into a delivery device or container under aseptic conditions.

36 A method according to any of aspects/embodiments/features 27 to 35 wherein the powder comprises an amorphous material, which may be a polymer, amino acid, protein or saccharide, such as trehalose, optionally wherein the material is substantially in glass form at 20 degrees C.

37 A sterile powder composition or pharmaceutical composition according to any preceding aspect/embodiment/feature or made by a method according to any preceding aspect/embodiment/feature, for use as a fibrin sealant or hemostat in the topical treatment of a wound, wherein the wound is selected from minor abrasions, cuts, scrapes, scratches, burns, sunburns, ulcers, internal venous or arterial bleeding, external venous or arterial bleeding, and surgical interventions selected from those involving the gastrointestinal system, on parenchymal organs; surgical interventions in the ear, nose and throat area (ENT) cardiovascular surgery, aesthetic surgery, spinal surgery, neurological surgery; lymphatic, biliary, and cerebrospinal (CSF) fistulae, air leakages during thoracic and pulmonary surgery, thoracic surgery, orthopaedic surgery; gynaecological surgical procedures; vascular surgery, liver resection, soft tissue injury and emergency surgery.

38 A pharmaceutical composition or powder composition according to any of aspects/embodiments/features 1 to 23 which comprises trehalose, optionally in an amount of at least 3 fold excess by weight of thrombin and, where present, at least 3 fold excess by weight of fibrinogen, optionally in 3 fold excess of the combined total of thrombin and fibrinogen.

39 A sterile powder composition suitable for medical use according to any of aspect/embodiment/features 1 to 25 comprising thrombin and fibrinogen, wherein the powder exhibits a thrombin potency or activity of at least 500 IU's per gram sterile powder composition, and/or a fibrinogen content of at least 50 mg per gram sterile powder composition, and/or wherein the powder comprises 60 to 130 mg protein per gram sterile powder composition, and/or wherein the powder comprises not more than 6% w/w residual water, and/or wherein said powder exhibits a crystalline purity of not less more than 15% w/w, and/or a median diameter between 10 and 100 microns, optionally when assessed as an average of at least 3 different batches, optionally up to 10 different batches, and wherein said pharmaceutical composition or powder composition additionally comprises an amorphous material, which material such as a polymer, amino acid, protein or saccharide, such as trehalose, or combination thereof, optionally wherein the material or combination of materials is substantially in glass form at 20 degrees C.

40 A pharmaceutical composition or a sterile powder composition suitable for medical use comprising thrombin, wherein the powder is produced from a feedstock, such as a liquid feedstock, and wherein the powder resulting from removal of liquid from the feedstock exhibits at least 80% of the thrombin activity or potency of the feedstock, and/or wherein the powder exhibits at least 500 IU's of thrombin potency or activity per gram sterile powder composition, optionally when assessed as an average of at least 3 different batches, optionally up to 10 different batches, and wherein said pharmaceutical composition or powder composition additionally comprises an amorphous material, such as a polymer, amino acid, protein or saccharide, such as trehalose, or combination thereof, optionally wherein the material or combination of materials is substantially in glass form at 20 degrees C., wherein the thrombin is provided in combination with fibrinogen, and/or wherein the powder comprises 60 to 130 mg protein/gram composition, and/or a fibrinogen content of at least 50 mg per gram sterile powder composition, and/or wherein the powder comprises not more than 6% w/w residual water, and/or wherein said powder exhibits a crystalline purity of not more than 15% w/w, and/or a median diameter between 10 and 100 microns.

41 A method for preparing a sterile powder thrombin composition, the method comprising removing water from an aqueous solution comprising thrombin, wherein removing water is carried out under aseptic conditions, optionally wherein the removal of water is by spray drying or fluid bed drying, and wherein the thrombin is provided in combination with fibrinogen, wherein fibrinogen is spray dried under aseptic conditions to form a powder, thrombin is separately spray dried under aseptic conditions to form a powder and the powders are then combined under aseptic conditions, wherein thrombin is comprised within any or all of the discrete units constituting said powder wherein fibrinogen is comprised within any or all of the discrete units constituting said powder, and the two powders are combined in a weight ratio of about 1:1, and wherein said pharmaceutical composition or powder composition additionally comprises an amorphous material, which may be a polymer, amino acid, protein or saccharide, such as trehalose, or combination thereof, optionally wherein the material or combination of materials is substantially in glass form at 20 degrees C., and wherein the powder exhibits at least 500 IU's of thrombin activity per gram sterile powder composition, and/or wherein the powder comprises 60 to 130 mg total protein/gram composition, and/or a fibrinogen content of at least 50 mg per gram sterile powder composition, and/or wherein the powder comprises not more than 6% w/w residual water, and/or wherein said powder exhibits a crystalline purity of not more than 15% w/w, and/or a median diameter between 10 and 100 microns, optionally when assessed as an average of at least 3 different batches, optionally up to 10 different batches.

42 A sterile powder composition suitable for medical use comprising thrombin wherein the powder composition elicits an anti-thrombin antibody immune response in fewer than 5% (4%, 3%, 2%, 10%) of patients.

43. A sterile powder composition or pharmaceutical composition according to aspect/embodiment/feature 42, optionally further comprising fibrinogen, for use as a hemostat in liver resection surgery or spinal surgery, wherein administration or medical use of said composition elicits an anti-thrombin antibody immune response in fewer than 3%, 2%, or 1% or less in a sample population of subjects, such as where there are at least 10, 20, 30, 40, 50, 75, 100, 125, 150, 200, 300, 400, 500 or more subjects.

44. A sterile powder composition or pharmaceutical composition comprising thrombin, optionally further comprising fibrinogen, for use as a hemostat in liver resection surgery, spinal surgery, soft tissue surgery or vascular surgery, wherein administration or medical use of said composition results in a median time to hemostasis (TTH) of less than about 2 minutes, or less, such as about 1.9 minutes, or about 1.5 minutes, or about 1.0 minute.

45. A sterile powder composition or pharmaceutical composition according to aspect/embodiment/feature 44, for use as a hemostat in liver resection surgery or spinal surgery, wherein administration or medical use of said composition results in a median time to hemostasis (TTH) of less than about 1.5 minutes, or about 1.0 minute, or less.

46. A sterile powder composition or pharmaceutical composition according to aspect/embodiment/feature 45, for use as a hemostat in liver resection, wherein administration or medical use of said composition results in a median time to hemostasis (TTH) of less than about 1.5 minutes, or less than about 1.2 minutes, or about 1.0 minute, or less.

47. A sterile powder composition or pharmaceutical composition comprising thrombin according to any of aspects/embodiments/features 1 to 25, optionally further comprising fibrinogen, for use in the treatment of bleeding associated with liver resection surgery, spinal surgery, soft tissue surgery or vascular surgery.

48. A sterile powder according to aspect/embodiment/feature 47 wherein said treatment of bleeding is associated with liver resection surgery or spinal surgery.

49. A sterile powder according to aspect/embodiment/feature 47 or 48 wherein said treatment of bleeding is associated with liver resection surgery.

50. A thrombin powder composition or pharmaceutical composition, optionally further comprising fibrinogen, prepared by removal of liquid from a solution or suspension of said thrombin or fibrinogen, wherein removing said liquid is carried out under aseptic conditions, optionally by spray drying, co-spray drying or fluid bed drying, for use in the treatment of bleeding associated with liver resection surgery, soft tissue surgery, vascular surgery or spinal surgery.

All concentrations and ratios of components disclosed herein are disclosed in respect of all starting powders, final compositions for human or animal use, and any intermediate compositions. In one aspect the concentrations and ratios relate to the final powder formulation for human or animal use. In another aspect the concentrations and ratios relate to a spray dried material produced from a feedstock without any further formulation steps.

In one aspect the concentrations and ratios of components disclosed herein are in respect of a composition comprising trehalose and fibrinogen, or trehalose and thrombin, or fibrinogen, thrombin and trehalose.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine study, numerous equivalents to the specific procedures described herein. Such equivalents are considered to be within the scope of this invention and are covered by the claims. All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference. The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps

The term “or combinations thereof” as used herein refers to all permutations and combinations of the listed items preceding the term. For example, “A, B, C, or combinations thereof is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

All documents referred to herein are incorporated by reference to the fullest extent permissible.

Any element of a disclosure is explicitly contemplated in combination with any other element of a disclosure, unless otherwise apparent from the context of the application.

Having now generally described the invention, the same will be more readily understood through reference to the following examples which are provided by way of illustration, and are not intended to be limiting of the present invention, unless specified.

### EXAMPLES

### Example 1

The present invention may be carried out in the following exemplary way: A Uni-Glatt laboratory spray-coater housed in a Grade A isolator can be charged with 800 grams non pareil seeds (mean particle size of 300 microns). Feedstock may be prepared by dissolving 7.38 g human fibrinogen in 165 ml water containing 27.51 g trehalose dihydrate, which may then be sprayed onto the fluidized cores (using 0.2 micron pre-filtered nitrogen as the process and atomisation gas) at an inlet temperature of 50.degree. C., an outlet temperature of 40.degree. C., and an atomization air pressure of 3.5 bar, to produce fibrinogen-coated cores. The Uni-Glatt laboratory spray-coater may be cleaned and then charged with 800 grams non pareil seeds. Feedstock may be prepared by dissolving 75,123 IU human thrombin in 165.3 ml water containing 1.15 g calcium chloride dihydrate and 50.73 g trehalose dihydrate, which may be sprayed onto the fluidized cores (using 0.2 micron pre-filtered nitrogen as the process and atomisation gas) at an inlet temperature of 50.degree. C., an outlet temperature of 40.degree. C., and an atomization air pressure of 3.5 bar, to produce thrombin-coated cores.

The coated cores may be then blended at the desired ratio using a tumble blender in an aseptic environment and then packaged in unit dose vials or similar packaging prior to administration.

### Example 2

The present invention may be carried out in the following exemplary way: spray dried solid fibrinogen:trehalose microparticles can be prepared by dissolving 73.8 g human fibrinogen in 1650 ml.sub.—water containing 275.1 g trehalose dihydrate. The resultant solution may then be spray dried via a Niro Mobile Minor™ spray dryer using the following operating parameters:

atomizer: stainless steel Newland Rotary Atomizer

inlet temperature: 160.degree. C.

feed rate: 8 g/minute

atomizer voltage: 10.0 V Outlet temperature: >80.degree. C.

The resultant powder had a particle size (×50, geometric diameter) of 47 μm and a moisture content (Karl Fisher) of 5.9%.

Spray dried solid thrombin:trehalose microparticles were prepared by dissolving 751,230 IU human thrombin in 1653 ml water containing 11.5 g calcium chloride dihydrate and 507.3 g trehalose dihydrate. The resultant solution may then be spray dried via a Niro Mobile Minor™ spray dryer using the same operating parameters.

### Example 3

Preparation of Trehalose Solution

Following the receipt and release of the drug substances and excipients is preparation of the trehalose solution which is used to reconstitute both the freeze dried thrombin and freeze dried fibrinogen. The grade of trehalose used is that which is approved as a pharmaceutical component for oral and diagnostic preparations.

Preparation of the trehalose solution was performed in a Grade C environment. The compositions of the trehalose solutions used during the preparation of the individual thrombin and fibrinogen solutions are shown in Table 1; the trehalose solution quantities relate to the manufacturing of 5 kg batch sizes of spray dried thrombin or spray dried fibrinogen. For each component, the trehalose or trehalose:calcium chloride solution was prepared in a sterilized vessel.

TABLE 1
Composition of Trehalose
Solvent for Fibrinogen and Thrombin

| | Composition of | | |
|---|---|---|---|
| | Fibrinogen | Thrombin | |
| | Trehalose | Trehalose | |
| Component | Solvent | Solvent | Standard |
| Trehalose | 3350 g | 4870 g¹ | USP |
| dihydrate | | | |
| Calcium chloride | 0 | 115 g | USP |
| dihydrate | | | |
| Water for Injection | 14.3 liters | 14.3 liters | USP |

¹corrected for moisture (10.5% from trehalose dihydrate CofA)

Fibrinogen and Thrombin Solutions for Spray Drying

The next step is the reconstitution of the fibrinogen and thrombin separately using the trehalose or trehalose: calcium chloride solutions outlined above. The reconstitution step was performed in a Grade C environment.

Fibrinogen Solution

Approximately 20 to 22 ml of the trehalose solution was added to each 1.0 gram fibrinogen raw material vial. The remainder of trehalose solution is transferred to a 50 liter vessel. The vials were placed on a shaker table to allow for complete reconstitution of the active. Once dissolution is complete, the contents of each vial were transferred to the 50 liter vessel containing the remaining trehalose solution. The resultant solution was sterile filtered into a separate 50 liter vessel located within the Class A isolator housing the aseptic spray dryer.

A total of 750 vials from one lot of fibrinogen drug substance (each vial of fibrinogen contains nominal 1 g of freeze dried fibrinogen) are reconstituted during the manufacturing of 5 kg batch, see Table 2.

TABLE 2
Fibrinogen (Quantities for
batch size of 5 kg of spray dried product)

| Component | No of Vials | Standard |
|---|---|---|
| Fibrinogen | 750 | Human |
| | | Fibrinogen |
| Trehalose Solution | Qs | |

¹Each vial contains 1 g freeze dried fibrinogen powder (nominal)

Thrombin Solution

Approximately 5 ml of the trehalose:CaCl.sub.2 solution was added to each vial of thrombin, the remainder of the trehalose solution is transferred to a 50 litre vessel. The vials were placed on a shaker table to allow for complete reconstitution of the active. Once dissolution is complete, the contents of each vial were transferred to the 50 liter vessel containing the remaining trehalose:CaCl.sub.2 solution.

The resultant solution was sterile filtered into a separate 50 liter vessel located within the Class A isolator housing the aseptic spray dryer.

A total of 150 vials from one lot of thrombin drug substance (each vial of thrombin contains nominal 50,000 IU freeze dried thrombin) are reconstituted to prepare a 5 Kg batch of spray dried thrombin, see Table 3.

TABLE 3
Thrombin (Quantities for batch
size of 5 Kg of spray dried product)

| Component | No of Vials | Standard |
|---|---|---|
| Thrombin | 150 | Human |
| | | Thrombin |
| Trehalose/CaCl₂ | qs | |
| Solution | | |

²Each vial contains 50,000 IU freeze dried thrombin powder (nominal)

Spray Drying

Fibrinogen and thrombin preparations were spray dried separately using an aseptic spray dryer housed in a Grade A isolator under positive pressure.

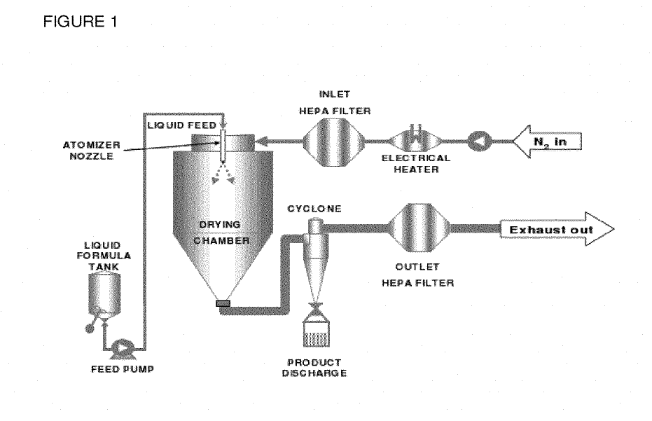

The spray dryer is configured with a cyclone collection system and a two-fluid atomization nozzle. The configuration of the spray drier is shown in FIG. 1 with the operating parameters in Table 4.

After a manufacturing run (change over between fibrinogen and thrombin) the spray dryer was cleaned.

TABLE 4
Spray Drying Operating Parameters

| | Fibrinogen | | | Thrombin | | |
|---|---|---|---|---|---|---|
| Operating | | Lower | Upper | | Lower | Upper |
| parameter | Target | Limit | Limit | Target | Limit | Limit |
| Inlet | 170 | 160 | 180 | 170 | 160 | 180 |
| temperature | | | | | | |
| (° C.) | | | | | | |
| Outlet | 90 | 80 | 100 | 90 | 80 | 100 |
| temperature | | | | | | |
| (° C.) | | | | | | |
| Atomization | 3.0 | 3.5 | 2.5 | 2.0 | 1.7 | 2.3 |
| Airflow | | | | | | |
| (Kg/hour) | | | | | | |
| Drying Air | 90 | 85 | 95 | 90 | 85 | 95 |
| Airflow | | | | | | |
| Kg/hour) | | | | | | |
| Solution | 2150 | 2050 | 2250 | 2325 | 2225 | 2425 |
| feed | | | | | | |
| Rate | | | | | | |
| (g/hour) | | | | | | |

On completion of spray drying, the collection vessels containing the spray dried product were sampled for QC, sealed inside the Grade A isolator and packaged in aluminum foil pouches. After sampling, the remaining spray dried product was stored until required for blending within a blend/fill Grade A isolator.

Blending of the Spray Dried Components

Post QC release, the spray dried fibrinogen and spray dried thrombin were weighed in a 1:1 ratio and the resultant powder was blended within a Grade A isolator until homogenous.

The blend was stored within the blending vessel until required for the filling operation.

Filling and Formulation/Labeling/Packaging

Sterilized glass vials were used as primary containers. Each vial was filled with 1 gram of blend and then each vial was manually stoppered and oversealed with a sterile tear off aluminum crimp seal.

### Example 4

Thrombin and Fibrinogen-containing powders were prepared according to the inlet/outlet temperature ranges of Example 3, except the atomiser employed was a two-fluid Schlick nozzle and the apparatus was a GEA Niro Mobile Minor™ housed in a non-aseptic GMP area, with subsequent exposure of filled vials to e-beam irradiation at 15 kGy). Numerous replicate batches were prepared, the conditions and results of which are shown in Tables 12 and 13, respectively.

As can be seen, both the spray-dried thrombin product and final irradiated product show increasing losses of thrombin potency/activity.

### Example 5

Further sterile powders were prepared in accordance with Example 3 above, with varying conditions and parameters, as shown in Tables 14 and 15, the analysis of said powders presented therein. As can be seen, the aseptically spray-dried thrombin product and product show minimal losses of thrombin potency/activity.

### Example 6

A number of further batches were prepared using a Niro Mobile Minor™ with cyclone collection and a Niro 2-fluid nozzle, Blends of the resultant spray dried powders were also manufactured and the spray dried batches and blends were subsequently analysed. The following batches were manufactured:

TABLE 6
Batch Manufacture

| Batch Number | Batch Details |
|---|---|
| 33#18/02 | Thrombin Drug Substance, 1500 IU/g (Sterile filtered |
| | thrombin solution (Extended Storage at 2-8° C., 9 days) |
| 33#18/03 | Thrombin drug substance, 1500 IU/g |
| 33#18/04 | Thrombin drug substance, 3000 IU/g |
| 33#18/05 | Fibrinogen Drug Substance, 100 mg/g |
| 31#18/06 | Fibrinogen Drug Substance, 150 mg/g |
| 33#19/01 | Blend 1: 750 IU/g thrombin: 75 mg/g fibrinogen |
| 33#19/02 | Blend 2: 2000 IU/g thrombin: 50 mg/g fibrinogen |
| 33#19/03 | Blend 3: 1500 IU/g thrombin: 75 mg/g fibrinogen |
| 33#19/04 | Blend 4: 1500 IU/g thrombin: 50 mg/g fibrinogen |

Each of the spray dried batches were manufactured using the target parameters shown below:

TABLE 7
Operating parameters

| Operating parameter | Target range | |
|---|---|---|
| Inlet temperature | 170° | C. |
| Outlet temperature | 80-90° | C. |
| Atomisation pressure | 0.8 | bar* |
| Atomisation airflow | 1-2 | L/sec |
| Solution feed rate | 14-18 | g/minute |

Each batch was collected in a glass jar which was sealed with parafilm and double bagged with desiccant.

Blending—The following blends (12 g batch size) were prepared:

TABLE 8
Blend Components

| Component | Blend 1 | Blend 2 | Blend 3 | Blend 4 |
|---|---|---|---|---|
| Thrombin 3000 IU/g | 3 g | 8 g | 6 g | 6 g |
| Thrombin 1500 IU/g | x | x | x | x |
| Fibrinogen 100 mg/g | 9 g | x | x | 6 g |
| Fibrinogen 150 mg/g | x | 4 g | 6 g | x |

Results

TABLE 9
Spray dried Thrombin Drug Substance

| | B/N 33#18/02 | B/N 33#18/03 | B/N 33#18/04 |
|---|---|---|---|
| Test | 1500 IU/g | 1500 IU/g | 3000 IU/g |
| Particle size (X50, | 20 | 17 | 15 |
| μm), | | | |
| Moisture content (%) | 4 | 4 | 3 |
| Thrombin Content | 1606* | 1679 | 3246 |
| (IU/g) | | | |

*Thrombin content as determined by Time-to-Clot method

TABLE 10
Spray Dried Fibrinogen Drug Substance

| | B/N 33#18/05 | B/N 33#18/06 |
|---|---|---|
| Test | 100 mg/g | 150 mg/g |
| Particle size (X50, μm), | 23 | 23 |
| Moisture content (%) | 3 | 3 |
| Fibrinogen Content | 102 | 149 |
| (mg/g) | | |

TABLE 11
Non-irradiated Drug Product (Blends)

| | B/N | B/N | B/N | B/N |
|---|---|---|---|---|
| | 33#19/01 | 33#19/02 | 33#19/03 | 33#19/04 |
| Test | Blend 1 | Blend 2 | Blend 3 | Blend 4 |
| Fibrinogen | 75 | 48 | 74 | 50 |
| Content | | | | |
| (mg/g) | | | | |
| Thrombin | 754 | 1978 | 1252 | 1590 |
| Content | | | | |
| (IU/g) | | | | |

The data generated confirms that the active content of the batches is very close to the target nominal for each batch, i.e. there is increased thrombin retention post spray drying.

### Example 7

To compare the immunogenicity of the thrombin component of the products of Example 4 (irradiated) versus Example 3 and Example 5 (non-irradiated, aseptic), a series of anti-thrombin ELISAs were developed under the principles of the Draft Guidance of Industry: Assay Development for Immunogenicity Testing of Therapeutic Proteins, December 2009, Guideline on Immunogenicity Assessment of Biotechnology-derived Therapeutic Proteins (CHMP July 2007), and validated under the principles of Guidance of Industry: BioAnalytical Method Validation (May 2001), as appropriate.

In addition, a neutralizing antibody assay was developed and validated to characterize whether any antibodies developed against the thrombin component of the product of the invention (known as Fibrocaps) which could neutralize the clotting activity of thrombin (human plasma-derived).

Human subjects were assessed for immunogenicity to the thrombin component of each product at Baseline (screening draw), and Day 29 (.+−0.4 days) after treatment using a tiered approach (see Table below for method details). The presence of binding antibodies (Abs) to thrombin was determined using a fully validated, direct anti-human thrombin antibody enzyme linked immunosorbant assay (ELISA). Any positive samples, i.e. subjects with a specific and measurable titer to human thrombin, were assessed in a fully validated assay to measure neutralizing potential of the antibodies.

### Assessment of Anti-Thrombin Antibodies for Examples 3 and 4

### Assessment of Anti-Thrombin Antibodies for Examples 3 and 4

| | Purpose of | |
|---|---|---|
| Tier | Assay | Description |
| 1 | Screening assay | “Detection of Anti-Thrombin Antibodies in Human Plasma by |
| | for binding | ELISA.” The basic format is as follows: microtiter plates coated |
| | antibodies¹ | with human thrombin are used to capture anti-human thrombin |
| | | antibodies present in 1/50 diluted study samples and controls. |
| | | Following a wash step, a cocktail of goat-anti-human |
| | | IgG + IgM/rabbit anti-Sheep IgG-HRP detection reagent is added |
| | | to complete the binding complex. TMB substrate is then used to |
| | | detect bound antibodies colorimetrically. A cut-point was |
| | | determined as the empirical 95th percentile (from the following |
| | | formula (Shankar, 2008; Mire-Sluis, 2004): N = 95th percentile - |
| | | Average NQC |
| 2 | Determine | Titre was evaluated by serially diluting to below the cut-point |
| | antibody titre¹ | level. The titer response was determined by fitting the dilution |
| | | profile (signal response versus dilution) to an appropriate |
| | | regression curve (4-PL) and interpolating the titre as 1/dilution at |
| | | the plate cut point × 50 × log₁₀. |
| 3 | Assess antibody | All samples with results greater than the plate-specific cut point |
| | specificity | and a measureable titre were assessed in the confirmatory |
| | | assay. Samples needed to show a reduction greater than |
| | | 38.2% inhibition to be considered specific for anti-thrombin |
| | | antibodies. |
| 4 | Assess | Samples that have a specific and measurable titre will |
| | neutralizing | subsequently be assessed in the neutralizing antibody assay. |
| | potential of anti- | Samples will be reported as positive for neutralizing antibodies if |
| | thrombin | a significant difference between pre-treatment (baseline) and |
| | antibodies | post-treatment (Day 29) is observed in thrombin time. |

Results

The product of Example 4 (with terminal irradiation) elicited a low, but detectable rate (5.2%) of antibody formation to thrombin (see Table 16). In comparison, the product manufactured aseptically (Example 3) elicited a much lower rate of antibody formation (0%) as evidenced in the 100 subjects assessed (Table 17). Neutralizing antibodies to thrombin were not observed in either of the products of Example 3 or 5. Taken together, these data suggest that improvements in the product manufacturing process, i.e. aseptic manufacture results in a reduced rate of immunogenicity.

TABLE 12
IRRADIATED PRODUCT-2-fluid Schlick atomiser

| | Stock thrombin conc (IU/ml) | | | | | | | | | | | | | |
|---|---|---|---|---|---|---|---|---|---|---|---|---|---|---|
| | 10547 | | | | | | | | | | 9900 | | 9900 | |
| # vials | 1 | | | | | | | | | | 1 | | 15 | |
| Total IU in | 52735 | | | | | | | | | | 49500 | | 742500 | |
| batch | | | | | | | | | | | | | | |
| Trehalose | 32.5 | | | | | | | | | | 32.5 | | 487 | |
| CaCl2 | 0.8 | | | | | | | | | | 0.8 | | 11.25 | |
| Thrombin | 1588 | | | | | | | | | | 1490 | | 1490 | |
| conc solids | | | | | | | | | | | | | | |
| (IU/g) | | | | | | | | | | | | | | |
| Atomization | 1.5 | 1.5 | 1.5 | 1.5 | 1.5 | 1.5 | 1.5 | 1.5 | 1.5 | 1.5 | 1.5 | 1.5 | 1.5 | 1.5 |
| Airflow | | | | | | | | | | | | | | |
| (L/sec) | | | | | | | | | | | | | | |
| Atomization | 0.5 | 0.4 | 0.6 | 0.6 | 0.4 | 0.5 | 0.6 | 0.6 | 0.3 | 0.3 | 0.5 | 0.4 | 0.4 | 0.4 |
| pressure (bar) | | | | | | | | | | | | | | |
| Feed rate | 960 | 840 | 840 | 1080 | 1080 | 960 | 840 | 1080 | 840 | 1080 | 1200 | 970 | 1040 | 960 |
| (g/hr) | | | | | | | | | | | | | | |
| Thrombin | 1365 | 1307 | — | — | 1179 | 1286 | — | — | 1048 | 1265 | — | 1415 | 1110 | 1213 |
| content | | | | | | | | | | | | | | |
| (IU/g)** | | | | | | | | | | | | | | |
| Ratio | 86% | 82% | — | — | 74% | 81% | — | — | 66% | 80% | — | 95% | 75% | 81% |
| output/actual | | | | | | | | | | | | | | |
| input (CSL) | | | | | | | | | | | | | | |
| Ratio | 91% | 87% | — | — | 79% | 86% | — | — | 70% | 84% | — | 94% | 74% | 81% |
| output/1500 | | | | | | | | | | | | | | |
| (target) | | | | | | | | | | | | | | |

| | Stock thrombin conc (IU/ml) | | | | | | | | | |
|---|---|---|---|---|---|---|---|---|---|---|
| | 9900 | | | | | | 9987/10449 | Mean | SD | STDDEV |
| # vials | 2 | | | | | | 15 | | | |
| Total IU in | 99000 | | | | | | 772125 | | | |
| batch | | | | | | | | | | |
| Trehalose | 65 | | | | | | 487 | | | |
| CaCl2 | 1.6 | | | | | | 11.25 | | | |
| Thrombin | 1486 | | | | | | 1550 | | | |
| conc solids | | | | | | | | | | |
| (IU/g) | | | | | | | | | | |
| Atomization | 1.0 | 1.0 | 1.9 | 1.5 | 1.4 | 1.5 | 1.5 | | | |
| Airflow | | | | | | | | | | |
| (L/sec) | | | | | | | | | | |
| Atomization | 0.3 | 0.5 | 0.5 | 0.4 | 0.3 | 0.4 | 0.3 | | | |
| pressure (bar) | | | | | | | | | | |
| Feed rate | 1080 | 840 | 1080 | 960 | 840 | 960 | 960 | | | |
| (g/hr) | | | | | | | | | | |
| Thrombin | 1197 | 1215 | 1124 | 1026 | 915 | 1114 | 1241 | 1167 | 129.6 | 11.1% |
| content | | | | | | | | | | |
| (IU/g)** | | | | | | | | | | |
| Ratio | 81% | 82% | 76% | 69% | 62% | 75% | 80% | 77% | 8% | 11% |
| output/actual | | | | | | | | | | |
| input (CSL) | | | | | | | | | | |
| Ratio | 80% | 81% | 75% | 68% | 61% | 74% | 83% | 78% | 9% | 11% |
| output/1500 | | | | | | | | | | |
| (target) | | | | | | | | | | |

**by Chromogenic assay

TABLE 13
RESULTS

| | | | | | | | | | | |
|---|---|---|---|---|---|---|---|---|---|---|
| Fibrinogen | 75 | 75 | 75 | 75 | 75 | 74 | 63 | 63 | 74 | 80 |
| content | | | | | | | | | | |
| (mg/g) | | | | | | | | | | |
| Thrombin | 373 | 419 | 408 | 413 | 404 | 492 | 446 | 453 | 436 | 440 |
| content | | | | | | | | | | |
| (IU/g) | | | | | | | | | | |
| Fibrinogen | | | | | | | | | | |
| Ratio | 49% | 49% | 49% | 49% | 49% | 54% | 46% | 46% | 42% | 51% |
| output/ | | | | | | | | | | |
| actual | | | | | | | | | | |
| input | | | | | | | | | | |
| (CSL) | | | | | | | | | | |
| Ratio | 100% | 100% | 100% | 100% | 100% | 99% | 84% | 84% | 99% | 107% |
| output/75 | | | | | | | | | | |
| (target) | | | | | | | | | | |
| Thrombin | | | | | | | | | | |
| Ratio | 23% | 26% | 26% | 26% | 25% | 33% | 30% | 30% | 29% | 30% |
| output/ | | | | | | | | | | |
| actual | | | | | | | | | | |
| input | | | | | | | | | | |
| (CSL) | | | | | | | | | | |
| Ratio | 50% | 56% | 54% | 55% | 54% | 66% | 59% | 60% | 58% | 59% |
| output/ | | | | | | | | | | |
| 750 | | | | | | | | | | |
| (target) | | | | | | | | | | |

RESULTS

| | | | | | | | Average | SD | STDEV |
|---|---|---|---|---|---|---|---|---|---|
| Fibrinogen | 82 | 86 | 84 | 81 | 79 | 76 | 76 | 6 | 8% |
| content | | | | | | | | | |
| (mg/g) | | | | | | | | | |
| Thrombin | 439 | 460 | 447 | 388 | 502 | 480 | 438 | 36 | 8% |
| content | | | | | | | | | |
| (IU/g) | | | | | | | | | |
| Fibrinogen | | | | | | | | | |
| Ratio | 52% | 55% | 54% | 52% | 50% | 50% | 50% | 3% | 7% |
| output/ | | | | | | | | | |
| actual | | | | | | | | | |
| input | | | | | | | | | |
| (CSL) | | | | | | | | | |
| Ratio | 109% | 115% | 112% | 108% | 105% | 101% | 101% | 8% | 8% |
| output/75 | | | | | | | | | |
| (target) | | | | | | | | | |
| Thrombin | | | | | | | | | |
| Ratio | 30% | 31% | 30% | 26% | 34% | 31% | 29% | 3% | 10% |
| output/ | | | | | | | | | |
| actual | | | | | | | | | |
| input | | | | | | | | | |
| (CSL) | | | | | | | | | |
| Ratio | 59% | 61% | 60% | 52% | 67% | 64% | 58% | 5% | 8% |
| output/ | | | | | | | | | |
| 750 | | | | | | | | | |
| (target) | | | | | | | | | |

TABLE 14
Two-fluid atomization nozzle (Niro design); Aseptic Spray Dryer

| | Concentration (IU/ml) | | | | | | | | | | |
|---|---|---|---|---|---|---|---|---|---|---|---|
| | 10449 | | | | 10449 | | | | | | |
| # vials | 23 | | | | 9 | | | | | | |
| Total IU in | 1201635 | | | | 470205 | | | | | | |
| batch | | | | | | | | | | | |
| Trehalose | 780.1 | | | | 292.3 | | | | | | |
| CaCl2 | 20.0 | | | | 6.8 | | | | | | |
| Thrombin | 1502 | | | | 1572 | | | | | | |
| concentration | | | | | | | | | | | |
| solids (IU/g) | | | | | | | | | | | |
| Atomization | 1.6 | 1.6 | 1.8 | 1.8 | 3 | 1.6 | 2.3 | 1.6 | 3 | 2.3 | 2.3 |
| Airflow (kg/hr) | | | | | | | | | | | |
| Atomization | 1 | 1.2 | 1 | 1.2 | 2.5 | 1.1 | 1.9 | 1 | 2.8 | 1.5 | 1.6 |
| Pressure (bar) | | | | | | | | | | | |
| resultant | | | | | | | | | | | |
| Feed rate | 2412 | 2460 | 2370 | 2495 | 2175 | 2456 | 2200 | 2100 | 2413 | 2208 | 2400 |
| (g/hr) | | | | | | | | | | | |
| Thrombin | 1447 | 1509 | 1509 | 1459 | 1548 | 1364 | 1452 | 1334 | 1518 | 1503 | 1504 |
| content (IU/g)** | | | | | | | | | | | |
| Ratio | 96% | 100% | 100% | 97% | 98% | 87% | 92% | 85% | 97% | 96% | 96% |
| output/actual | | | | | | | | | | | |
| input (CSL) | | | | | | | | | | | |
| Ratio | 96% | 101% | 101% | 97% | 103% | 91% | 97% | 89% | 101% | 100% | 100% |
| output/1500 | | | | | | | | | | | |
| (target) | | | | | | | | | | | |
| 1500 | | | | | | | | | | | |

| | Concentration (IU/ml) | | | | | | | |
|---|---|---|---|---|---|---|---|---|
| | 10449 | 11430 | 10449 | | | Average | SD | STDEV |
| # vials | 75 | 150 | 31 | | | | | |
| Total IU in | 3918375 | 8572500 | 1619595 | | | | | |
| batch | | | | | | | | |
| Trehalose | 2564.8 | 5230.6 | 1010 | | | | | |
| CaCl2 | 58.8 | 117.6 | 23.3 | | | | | |
| Thrombin | 1494 | 1603 | 1567 | | | | | |
| concentration | | | | | | | | |
| solids (IU/g) | | | | | | | | |
| Atomization | 2 | 2.3 | 1.7 | 1.9 | 1.9 | | | |
| Airflow (kg/hr) | | | | | | | | |
| Atomization | 1.3 | 1.1 | 1 | 0.9 | 0.9 | | | |
| Pressure (bar) | | | | | | | | |
| resultant | | | | | | | | |
| Feed rate | 2350 | 2325 | 2350 | 2320 | 2325 | | | |
| (g/hr) | | | | | | | | |
| Thrombin | 1585.75 | 1531 | 1528 | 1509 | 1473 | 1486 | 64.48 | 4.3% |
| content (IU/g)** | | | | | | | | |
| Ratio | 106% | 96% | 97% | 96% | 94% | 96% | 5% | 5% |
| output/actual | | | | | | | | |
| input (CSL) | | | | | | | | |
| Ratio | 106% | 102% | 102% | 101% | 98% | 99% | 4% | 4% |
| output/1500 | | | | | | | | |
| (target) | | | | | | | | |
| 1500 | | | | | | | | |

**by Chromogenic assay

TABLE 15
RESULTS

| | | | | | | | | | Average | SD | STDEV |
|---|---|---|---|---|---|---|---|---|---|---|---|
| Fibrinogen content (mg/g) | 73 | 74 | 76 | 67 | 67 | 69 | 68 | 73 | 71 | 4 | 5% |
| Thrombin content (IU/g)) | 654 | 602 | 671 | 683 | 607 | 677 | 663 | 681 | 655 | 33 | 5% |
| Fibrinogen | | | | | | | | | | | |
| Ratio output/actual input | 48% | 49% | 50% | 44% | 44% | 46% | 45% | 49% | 47% | 2% | 5% |
| (CSL) | | | | | | | | | | | |
| Ratio output/75 (target) | 97% | 99% | 101% | 89% | 89% | 92% | 91% | 97% | 95% | 5% | 5% |
| Thrombin | | | | | | | | | | | |
| Ratio output/actual input | 44% | 40% | 45% | 43% | 39% | 45% | 41% | 43% | 43% | 2% | 5% |
| (CSL) | | | | | | | | | | | |
| Ratio output/750 (target) | 87% | 80% | 89% | 91% | 81% | 90% | 88% | 91% | 87% | 4% | 5% |

TABLE 16
Anti-Thrombin Ab Results for Example 4

| | Anti-Thrombin | Anti-Thrombin |
|---|---|---|
| | Abs n/N (%): | Abs n/N (%): |
| | FC + Gelfoam Treated | Gelfoam Treated |
| | Subjects | Subjects |
| Antibody Positive¹ | 4/77 (5.2%) | 0/37 |
| Seroconversion² | 4/77 (5.2%) | N/A |
| ≥1.0 unit Titre Change³ | 0/77 (0%) | N/A |

¹Subjects are considered antibody positive if they have seroconverted (i.e. negative at baseline with a specific and measurable titre at day 29) or had a greater than 1.0 titre unit change at Day 29
²Subjects who were negative (either “Not Reactive” (NR) or Not Specific (NS)) at baseline 5 with a specific and measureable titre at Day 29
³Subjects who with a specific and measureable titre at baseline and a specific and measureable titre at Day 29 with a ≥ 1.0 unit titre change

TABLE 17
Anti-Thrombin Ab Results for Example 3

| | Anti-Thrombin | Anti-Thrombin |
|---|---|---|
| | Abs n/N (%): | Abs n/N |
| | FC + Gelfoam Treated | (%): Gelfoam Treated |
| | Subjects | Subjects |
| Antibody Positive¹ | 0/65 (0%) | 3/34 (9%) |
| Seroconversion² | 0/65 (0%) | 3/34 (9%) |
| ≥1.0 unit litre Change³ | 0/65 (0%) | 0/34 (0%) |

¹Subjects, are considered antibody positive if they have seroconverted (i.e. negative at baseline with a specific and measurable titre at day 29) or had a greater than 1.0 titre unit change at Day 29
²Subjects who were negative at baseline with a specific and measureable titre at Day 29
³Subjects who with a specific and measureable titre at baseline and a specific and measureable titre at Day 29 with a 1.0 unit titre change
⁴Soft-tissue subjects

TABLE 17A
Anti-Thrombin Ab Results for Example 3 in larger studies

| | Anti-Thrombin Abs n/N | Anti-Thrombin Abs n/N |
|---|---|---|
| | (%): FC + Gelfoam | (%): Gelfoam Treated |
| | Treated Subjects | Subjects |
| Antibody Positive¹ | 9/440 (2%) | 6/222 (3%) |
| Seroconversion² | 9/440 (2%) | 6/222 (3%) |
| ≥1.0 unit Titre | 0/440 (0%) | 0/222 (0%) |
| Change³ | | |

¹Subjects are considered antibody positive if they have seroconverted (i.e. negative at baseline with a specific and measurable titer at day 29) or had a greater than 1.0 titer unit change at Day 29
²Subjects who were negative at baseline with a specific and measureable titer at Day 29
³Subjects who with a specific and measureable titer at baseline and a specific and measureable titer at Day 29 with a ≥ 1.0 unit titer change

The product manufactured aseptically (Example 3) elicited a low rate of antibody formation (2%) as evidenced in the 440 subjects assessed and is less than the antibody formation elicited when applying a control hemostatic gelatin sponge devoid of thrombin to 222 subjects (Table 17A). Taken together, these data suggest that improvements in the product manufacturing process, i.e. aseptic manufacture, results in a reduced rate of immunogenicity equivalent to or superior to that observed with a thrombin-free control.

What is claimed is:

1. A method for preparing sterile powder composition suitable for medical use comprising a thrombin powder and a fibrinogen powder, wherein the thrombin powder is produced from a liquid feedstock, wherein the feedstock comprises a solution of thrombin, wherein the thrombin powder is produced by removal of liquid by aseptic spray drying, and wherein the thrombin powder resulting from removal of liquid from the feedstock exhibits at least 80% of the thrombin potency or activity of the liquid feedstock, and wherein the fibrinogen powder is produced by removal of liquid from a feedstock by aseptic spray drying, wherein the feedstock comprises a solution of fibrinogen, wherein the aseptic spray dryer is housed in a Grade A isolator under nitrogen, the sterile powder composition exhibits at least 600 IU's of thrombin potency or activity per gram of the sterile powder composition, and wherein said composition is not irradiated.

2. The method according to claim 1 comprising an additional pharmaceutically active agent.

3. The method according to claim 1 comprising:
(i) a composite particle comprising thrombin and fibrinogen, or
(ii) a mixture of particles that comprise fibrinogen in the absence of thrombin, and particles that comprises thrombin in the absence of fibrinogen,
wherein (i) or (ii) are in the form of nanoparticles, nanofibres, fibres, particles, granules, beads, microbeads, microspheres, microcapsules or microparticles.

4. The method according to claim 1 which exhibits at least 1300 IU's of thrombin potency or activity per gram sterile thrombin powder composition.

5. The method according to claim 1, wherein thrombin activity is measured by a chromogenic assay or wherein potency is measured by a time to clot assay.

6. The method according to claim 1, wherein the powder resulting from the removal of the liquid from the feedstock exhibits at least 85% of the thrombin activity or potency, or exhibits at least 85% of the fibrinogen activity or potency, or exhibits at least 85% of both the thrombin and fibrinogen activity or potency relative to their respective feedstock.

7. The method according to claim 1, wherein the liquid present in the feedstock is selected from an aqueous or organic solvent, or mixtures thereof.

8. The method according to claim 1, wherein the degree of crystallinity as measured by x-ray powder diffraction (XRPD) or Fourier transform infrared spectroscopy (FTIR) is not greater than 15% w/w

9. The method according to claim 1, comprising no more than 20% w/w residual water or moisture, optionally no more than 10% w/w residual water or moisture by weight, optionally no more than 6% w/w residual water or moisture.

10. The method according to claim 1, comprising 60 to 130 mg protein/gram composition.

11. The method according to claim 1, comprising one or more amorphous materials, selected from: a polymer, amino acid, protein or saccharide, and combinations thereof.

12. The method of claim 11, wherein one or more of the amorphous materials is trehalose.

13. The method according claim 1, wherein either or both of said thrombin or fibrinogen powders or a composite thereof, comprise trehalose, optionally present in an excess of the amount of thrombin by weight, or optionally present in an excess of the amount of fibrinogen by weight, or both.

14. The method according to claim 1 for use in dry powder topical application.

15. The method according to claim 1 wherein said feedstock is made by dissolving or suspending in a liquid a thrombin-containing solid having an activity or potency of 1500 IU/gram solid to produce the feedstock.

16. A method for preparing a sterile powder composition comprising fibrinogen and thrombin, wherein the thrombin powder is produced from a liquid feedstock, wherein the feedstock comprises a solution or a suspension of thrombin, preferably a solution, wherein the powder is produced by removal of liquid by a process selected from aseptic spray drying or aseptic fluid bed drying, and wherein the powder resulting from removal of liquid from the feedstock exhibits at least 80% of the thrombin potency or activity of the liquid feedstock, and wherein the fibrinogen powder is produced by removal of liquid from a feedstock, wherein the feedstock comprises a solution or a suspension of fibrinogen, preferably a solution, by aseptic spray drying or aseptic fluid bed drying, optionally wherein the composition comprises:
(i) a composite particle comprising thrombin and fibrinogen, or
(ii) a mixture of particles that comprise fibrinogen in the absence of thrombin, and particles that comprises thrombin in the absence of fibrinogen,
optionally wherein (i) or (ii) are in the form of nanoparticles, nanofibres, fibres, particles, granules, beads, microbeads, microspheres, microcapsules or microparticles, preferably microparticles, and optionally wherein the composition is packaged as a sterile final pharmaceutical product for medical use, such as for direct topical application as a dry powder.

17. A method for preparing sterile powder composition suitable for medical use comprising combining a thrombin powder and a fibrinogen powder, wherein the thrombin powder is produced from a liquid feedstock, wherein the feedstock comprises a solution of thrombin, wherein the thrombin powder is produced by removal of liquid by aseptic spray drying, and wherein the thrombin powder resulting from removal of liquid from the feedstock exhibits at least 80% of the thrombin potency or activity of the liquid feedstock, and wherein the fibrinogen powder is produced by removal of liquid from a feedstock by aseptic spray drying, wherein the feedstock comprises a solution of fibrinogen, wherein the sterile powder composition exhibits at least 600 IU's of thrombin potency or activity per gram of the sterile powder composition, and wherein said composition is not irradiated.
